(19) United States
(12) Patent Application Publication
(10) Pub. No.: US 2026/0055412 A1
(43) Pub. Date: Feb. 26, 2026

(54) SIRNA TARGETING ANGPTL4, AND CONJUGATES AND USES THEREOF

(71) Applicant: LEADERNA THERAPEUTICS LTD., Sichuan (CN)

(72) Inventors: Zhaogui LIU, Sichuan (CN); Zhao WANG, Sichuan (CN); Jiehua ZHOU, Sichuan (CN); Jinqiao WAN, Sichuan (CN)

(21) Appl. No.: 19/185,311

(22) Filed: Apr. 22, 2025

(30) Foreign Application Priority Data
Apr. 26, 2024 (CN) 202410512777.X

(51) Int. Cl. C12N 15/113

(52) U.S. Cl. CPC C12N 15/1136; C12N 2310/14; C12N 2310/312; C12N 2310/315; C12N 2310/321; C12N 2310/322; C12N 2310/351

(57) ABSTRACT

The present invention provides an siRNA that inhibits angiopoietin-like 4 (ANGPTL4) gene expression, wherein the siRNA comprises a sense strand and an antisense strand, wherein the antisense strand comprises at least 17 continuous nucleotides that differ by no more than 4 nucleotides from the nucleotide sequences as shown in SEQ ID NO: 143, and the antisense strand is 17 to 30 nucleotides in length; the sense strand is 17 to 30 nucleotides in length and is at least partially complementary to the antisense strand. The siRNA, siRNA conjugate, and pharmaceutical composition provided in the present invention exhibit good stability, ANGPLT4 gene inhibitory activity, and immunostimulation, and can significantly reduce ANGPTL4 protein concentrations at the animal level.

### CROSS-REFERENCE TO RELATED APPLICATIONS

This application claims the benefit of priority under 35 U.S.C. § 119(a) to Chinese Patent Application No. 202410512777.X, filed on Apr. 26, 2024, the entire contents of which are hereby incorporated by reference.

### REFERENCE TO SEQUENCE LISTING

The instant application contains a Sequence Listing as an XML file entitled “BSP25431887US SEQ” created on Apr. 21, 2025 and having a size of 3,082 bytes.

### TECHNICAL FIELD

The present disclosure relates to an siRNA that inhibits angiopoietin-like 4 (ANGPTL4) gene expression, a conjugate thereof, a pharmaceutical composition thereof, and use thereof in the prevention and/or treatment of diseases related to dyslipidemia.

### BACKGROUND

Angiopoietin-like 4 (ANGPTL4), a member of the angiopoietin-like family, is a secreted protein primarily expressed in adipose and liver tissues before being secreted into the bloodstream. ANGPTL4 consists of 406 amino acids and mainly contains two functional domains: an N-terminal coiled-coil domain and a C-terminal fibrinogen-like domain, both of which are conserved domains of angiopoietin family members. ANGPTL4 commonly exists as oligomers, glycosylated forms, and various subtypes. In addition, ANGPTL4 also contains one asparagine glycosylation site, one cAMP/cGMP-dependent protein kinase phosphorylation site, two protein kinase C phosphorylation sites, four myristoylation sites, and four casein kinase II phosphorylation sites. Its expression is regulated by factors such as transforming growth factor-β (TGF-β), peroxisome proliferator-activated receptor δ (PPARδ), and hypoxia-inducible factor 1 alpha (HIF1α).

Growing evidence indicates that the biological function of ANGPTL4 plays a pivotal role in the pathogenesis of metabolic disorders, such as atherosclerosis, type 2 diabetes, fatty liver disease, and obesity. Notably, it can play a pivotal role in lipid metabolism in the blood and liver by inhibiting lipoprotein lipase (LPL) activity. Abnormal lipid metabolism in the liver leads to accumulation of triacylglycerol (TAG) and 1,2-diacylglycerol (DAG), thereby activating the PKCε signaling pathway, which suppresses insulin receptor activation and reduces hepatic insulin sensitivity. The decrease in hepatic insulin sensitivity inhibits the conversion of blood glucose into hepatic glycogen and promotes the dissimilation of glycogen into glucose, leading to elevated blood glucose levels. Multiple studies have demonstrated that ANGPTL4 expression is associated with lipid and glucose metabolism in vivo.

Type 2 diabetes and its complications exhibit a high incidence worldwide. According to the International Diabetes Federation, approximately 537 million adults (10%) are currently living with diabetes, a figure projected to rise to 643 million by 2023 and 783 million by 2045, 90% of whom have type 2 diabetes. In China, the Guidelines for the Prevention and Treatment of Type 2 Diabetes in China (2020 Edition) reported that the incidence of type 2 diabetes has risen to 11.2%. Despite continuous introduction of drugs for the treatment of type 2 diabetes in recent years, clinical glycemic control and patient compliance are still unsatisfactory. A research study published in the British Medical Journal in 2020 revealed that 49% of patients with type 2 diabetes received treatment in China, with merely 49.4% achieving glycated hemoglobin (HbA1c) targets. Moreover, existing drugs for the treatment of type 2 diabetes require a high frequency of administration, and most of the drugs need to be administered daily or even per meal, highlighting unmet needs for improved patient compliance.

Compared to conventional drugs, siRNA has the disadvantage of poor stability and susceptibility to nuclease degradation during systemic administration. Furthermore, attempts need to be made to avoid side effects such as off-target effects, immunostimulation, and cytotoxicity while further improving activity. Thus, there is an urgent need to develop more candidate siRNAs that are stable in the blood, have good bioactivity and low cytotoxicity, and can inhibit ANGPTL4 gene expression in a long-lasting manner. Concurrently, it is of necessity for clinical research and commercial feasibility to develop drugs that can effectively prevent and/or treat diseases related to abnormalities in lipid and glucose metabolism by utilizing these candidate siRNAs that inhibit ANGPTL4 gene expression.

### SUMMARY

The present disclosure provides a small interfering RNA (siRNA) preparation targeting ANGPTL4. By specifically binding to ANGPTL4 mRNA, the siRNA preparation is capable of disrupting normal translational templating of ANGPTL4 mRNA, which prevents the translation of ANGPTL4 protein, thus alleviating the inhibition of LPL activity, lowering TAG and DAG levels, attenuating the PKCε signaling pathway, enhancing insulin sensitivity, and improving lipid and glucose metabolism.

In one aspect, the present disclosure provides an siRNA that inhibits ANGPTL4 gene expression, wherein the siRNA comprises a sense strand and an antisense strand, wherein the antisense strand comprises at least 17 continuous nucleotides that differ by no more than 4 nucleotides from the nucleotide sequence as shown in any one of SEQ ID NOs: 1 to 211 and 663 to 665, and the antisense strand is 17 to 30 nucleotides in length; the sense strand is 17 to 30 nucleotides in length and is at least partially complementary to the antisense strand. In some embodiments, the antisense strand is 19 to 27 nucleotides in length, and the sense strand is 19 to 25 nucleotides in length.

In some embodiments, the antisense strand is 19 to 23 nucleotides in length, and the sense strand is 19 to 21 nucleotides in length.

In some embodiments, the antisense strand is 23 nucleotides in length, and the sense strand is 21 nucleotides in length. In some embodiments, the antisense strand is 22 nucleotides in length, and the sense strand is 20 nucleotides in length. In some embodiments, the antisense strand is 21 nucleotides in length, and the sense strand is 21 nucleotides in length. In some embodiments, the antisense strand is 21 nucleotides in length, and the sense strand is 19 nucleotides in length. In some embodiments, the antisense strand is 20 nucleotides in length, and the sense strand is 22 nucleotides in length. In some embodiments, the antisense strand is 20 nucleotides in length, and the sense strand is 20 nucleotides in length. In some embodiments, the antisense strand is 27 nucleotides in length, and the sense strand is 25 nucleotides in length. In some embodiments, the antisense strand is 19 nucleotides in length, and the sense strand is 19 nucleotides in length.

In some embodiments, the siRNA comprises an overhang of one or more single-stranded nucleotides, such as an overhang of 1, 2, 3, or 4 nucleotides. In some embodiments, the overhang may be present on the sense strand, the antisense strand, or any combination thereof. In some embodiments, the overhang may be present at the 5′ end, the 3′ end, or both ends of the antisense or sense strand of the siRNA.

In some embodiments, the siRNA comprises an overhang of 2 nucleotides at the 3′ end of the antisense strand.

In some embodiments, the siRNA comprises an overhang of 2 nucleotides at the 3′ end of the antisense strand, and the overhang is UU or GG.

In some embodiments, the siRNA comprises a blunt end. In some embodiments, the siRNA comprises at least one blunt end at the 5′ end of the antisense strand (or the 3′ end of the sense strand).

In some embodiments, the siRNA comprises two blunt ends.

In some embodiments, the antisense strand differs by no more than 4 nucleotides from any one of the nucleotide sequences as shown in SEQ ID NOs: 1 to 211 and 663 to 665. In some embodiments, the antisense strand differs by no more than 3 nucleotides from any one of the nucleotide sequences as shown in SEQ ID NOs: 1 to 211 and 663 to 665. In some embodiments, the antisense strand differs by no more than 2 nucleotides from any one of the nucleotide sequences as shown in SEQ ID NOs: 1 to 211 and 663 to 665. In some embodiments, the antisense strand differs by no more than 1 nucleotide from any one of the nucleotide sequences as shown in SEQ ID NOs: 1 to 211 and 663 to 665. In some embodiments, the antisense strand is any one of the nucleotide sequences as shown in SEQ ID NOs: 1 to 211 and 663 to 665.

In some embodiments, the sense strand has a mismatch of no more than 3 nucleotides with the antisense strand. In some embodiments, the sense strand has a mismatch of no more than 2 nucleotides with the antisense strand. In some embodiments, the sense strand has a mismatch of no more than 1 nucleotide with the antisense strand. In some embodiments, the sense strand is fully complementary to the antisense strand.

In some embodiments, there is an overhang of 2 nucleotides at the 3′ end of the sense strand. In some embodiments, the two nucleotides are reverse complementary to the first two nucleotides at the corresponding position of the starting nucleotide of the sense strand in the transcript as shown in NCBI Accession No. NM 139314.3.

In some embodiments, the sequence of the siRNA is selected from the sequences of duplex 1 to duplex 217.

In some embodiments, the sequence of the siRNA is selected from the sequences of duplex 143, duplex 144, duplex 146, duplex 147, duplex 148, duplex 149, duplex 209, duplex 210, duplex 211, duplex 212, duplex 213, duplex 214, duplex 215, duplex 216, and duplex 217.

In some embodiments, the siRNA comprises at least one modified nucleotide.

In some embodiments, all of the nucleotides in the sense strand and/or the antisense strand of the siRNA are modified nucleotides or nucleotide analogs.

In some embodiments, the modified nucleotide or nucleotide analog is selected from a 2′-methoxy nucleotide, a 2′-fluoro nucleotide, a 2′-deoxy nucleotide, a 2′,3′-seco nucleotide analog, a 2′-fluoroarabino nucleotide, a 2′-methoxyethyl nucleotide, a 2′-amino-modified nucleotide, a 2′-alkyl-modified nucleotide, a 3′-methoxy nucleotide, a 2′-allyl-modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol-modified nucleotide, an abasic nucleotide, a morpholino nucleotide, a threose nucleotide, a locked nucleotide, an unlocked nucleotide, a glycerol nucleotide, or a base-modified nucleotide.

In some embodiments, the 5′ and 3′ ends of the sense strand independently comprise 1 or 2 phosphorothioate linkages, respectively; and/or the 5′ and 3′ ends of the antisense strand independently comprise 1 or 2 phosphorothioate linkages, respectively.

In some embodiments, there is a phosphorothioate linkage between nucleotides at positions 1 and 2 from the 5′ end of the sense strand, between nucleotides at positions 2 and 3 from the 5′ end of the sense strand, between nucleotides at positions 1 and 2 from the 3′ end of the antisense strand, between nucleotides at positions 2 and 3 from the 3′ end of the antisense strand, between nucleotides at positions 1 and 2 from the 5′ end of the antisense strand, and between nucleotides at positions 2 and 3 from the 5′ end of the antisense strand.

In some embodiments, the antisense strand of the siRNA is 23 nucleotides in length, wherein the antisense strand comprises 3 to 10 2′-fluoro nucleotides (preferably at positions 2, 14, and 16; positions 2, 5, 14, and 16; positions 2, 6, 14, and 16; positions 2, 4, 6, 14, and 16; positions 2, 6, 9, 14, and 16; positions 2, 5, 6, 14, and 16; positions 2, 6, 10, 14, and 16; positions 2, 6, 12, 14, and 16; positions 2, 5, 10, 14, and 16; positions 2, 3, 12, 14, and 16; positions 2, 9, 12, 14, and 16; positions 2, 6, 8, 9, 14, and 16; positions 2, 3, 5, 12, 14, and 16; positions 2, 8, 9, 12, 14, and 16; positions 2, 7, 9, 12, 14, and 16; positions 2, 4, 6, 8, 10, 14, 16, 18 and 20; or positions 2, 4, 5, 6, 8, 10, 12, 14, 16, and 18 from the 5′ end), with the remaining positions being 2′-methoxy nucleotides; and the sense strand of the siRNA is 21 nucleotides in length, wherein positions 9, 10, and 11; positions 7, 9, 10, and 11; or positions 5, 7, 9, 10, and 11 from the 5′ end of the sense strand are 2′-fluoro nucleotides, optionally comprising a threose nucleotide at one position (preferably at position 1 from the 5′ end), optionally comprising a 2′-deoxy nucleotide at one position (preferably at position 7 from the 5′ end), with the remaining positions being 2′-methoxy nucleotides. In some embodiments, the antisense strand of the siRNA is the nucleotide sequence as shown in SEQ ID NO: 143, and the sense strand of the siRNA is the nucleotide sequence as shown in SEQ ID NO: 354.

In some embodiments, the antisense strand of the siRNA is 21 nucleotides in length, wherein the antisense strand comprises 3 to 10 2′-fluoro nucleotides (preferably at positions 2, 6, 14, and 16 from the 5′ end), and the remaining positions are 2′-methoxy nucleotides; the sense strand is 19 nucleotides in length, wherein positions 7, 8, and 9 from the 5′ end of the sense strand are 2′-fluoro nucleotides.

The antisense strand of the siRNA is 22 nucleotides in length, wherein the antisense strand comprises 3 to 10 2′-fluoro nucleotides (e.g., at positions 2, 6, 14, and 16 from the 5′ end), and the remaining positions are 2′-methoxy nucleotides; the sense strand is 20 nucleotides in length, wherein positions 6, 8, 9, and 10, or positions 8, 9, and 10 from the 5′ end are 2′-fluoro nucleotides.

In some embodiments, the first nucleotide at the 5′ end of the antisense strand is an (E)-vinylphosphonate-modified nucleotide.

In some embodiments, the sequence of the siRNA is selected from the sequence of one of the modified duplexes 1 to 21 as shown in Table 3.

In one aspect, the present disclosure further provides an siRNA conjugate, wherein the siRNA conjugate comprises the siRNA according to the present disclosure and a conjugate molecule.

In some embodiments, the siRNA conjugate comprises a linker-targeting ligand. In some embodiments, the targeting ligand comprises N-acetylgalactosamine. In some embodiments, the linker-targeting ligand is GalNAc (L96).

In some embodiments, the siRNA conjugate is selected from conjugate 1 to conjugate 242.

In some embodiments, the siRNA conjugate is selected from conjugate 1, conjugate 5, conjugate 15, conjugate 16, conjugate 17, conjugate 19, conjugate 21, conjugate 22, conjugate 23, conjugate 28, conjugate 92, conjugate 93, conjugate 120, conjugate 130, conjugate 133, conjugate 225, conjugate 227, conjugate 234, conjugate 235, conjugate 236, and conjugate 238.

In one aspect, the present disclosure further provides a method for preparing a conjugate, comprising the step of conjugating the siRNA according to the present disclosure with a conjugate molecule, thereby obtaining the conjugate.

In one aspect, the present disclosure further provides a pharmaceutical composition, comprising the siRNA according to the present disclosure and/or the siRNA conjugate according to the present disclosure, as well as a pharmaceutically acceptable carrier.

In some embodiments, the pharmaceutical composition further comprises a second therapeutic agent; more preferably, the second therapeutic agent is an oligonucleotide; further preferably, the second therapeutic agent is administered in the same or a different medicament as the siRNA or the conjugate.

In one aspect, the present disclosure provides a use of the siRNA, siRNA conjugate, and/or pharmaceutical composition according to the present disclosure in the manufacture of a medicament for treating and/or preventing a pathological condition or disease associated with overexpression of angiopoietin-like 4 (ANGPTL4) gene.

In some embodiments, the pathological condition or disease is a disease associated with dyslipidemia. In some embodiments, the disease associated with dyslipidemia is hyperlipidemia, hypertriglyceridemia, pancreatitis, familial chylomicronemia syndrome, diabetes, type 2 diabetes, heart disease, myocardial infarction, angina, or atherosclerosis.

The siRNA, siRNA conjugate, and pharmaceutical composition provided in the present disclosure exhibit good stability, excellent ANGPLT4 gene inhibitory activity, satisfactory cytotoxicity and immunostimulation, and can significantly reduce blood lipid levels.

In some embodiments, the ANGPLT4 gene targeted by the siRNA of the present disclosure has the sequence as shown in NCBI Accession No. NM 139314.3.

In the present disclosure, “siRNA” refers to an RNA or RNA-like (e.g., chemically modified RNA) oligonucleotide molecule capable of reducing or inhibiting the translation of a messenger RNA (mRNA) in a sequence-specific manner. The siRNA may function through an RNA interference mechanism (e.g., by inducing mRNA degradation via interaction with an mRNA interference pathway mechanism (RNA-induced silencing complex RISC) in mammalian cells), or any other mechanism or pathway. Although the term “siRNA drug” as used in the present disclosure is believed to primarily function through an RNA interference mechanism, the siRNA drug is not limited or restricted to any specific pathway or mechanism of action. siRNA drugs include, but are not limited to, single-stranded antisense oligonucleotides, small interfering RNA (siRNA), double-stranded RNA (dsRNA), microRNA (miRNA), short hairpin RNA (shRNA), and Dicer enzyme substrates. The siRNA drug according to the present disclosure consists of an oligonucleotide strand that is at least partially complementary to a target mRNA. In some embodiments, the siRNA drug according to the present disclosure is double-stranded and consists of an antisense strand and a sense strand that is at least partially complementary to the antisense strand.

The term “sequence” or “nucleotide sequence” refers to the order or arrangement of nucleobases or nucleotides, represented alphabetically using standard nucleotide nomenclature.

In the present disclosure, unless otherwise specified, C, G, U, A, and T represent the base composition of a nucleotide, including both modified and unmodified nucleotides; m represents that the nucleotide immediately to the right of the symbol m is a 2′-methoxy nucleotide; f represents that the nucleotide immediately to the right of the symbol f is a 2′-fluoro nucleotide; lowercase d represents that the nucleotide immediately to the right of the symbol d is a deoxyribonucleotide; gn represents that the nucleotide immediately to the right of the symbol gn is a glycerol nucleotide (GNA); tn represents that the nucleotide immediately to the right of the symbol tn is a threose nucleotide (TNA); symbol * represents that the two adjacent nucleotides flanking the symbol * are linked via a phosphorothioate linkage; eVP represents that the nucleotide immediately to the right is an (E)-vinylphosphonate-modified nucleotide; iab represents an inverted abasic residue. GalNAc (L96) refers to conjugation with the linker-targeting ligand moiety GalNAc (L96) at this position. Ser (GN) refers to conjugation with the linker-targeting ligand moiety Ser (GN) at this position.

In the present disclosure, unless otherwise specified, uppercase I represents the base composition of a base-modified nucleotide, wherein the base is

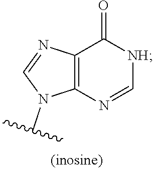 [SMILES: *n1cnc2c(=O)[nH]cnc21]

mI refers to inosine with a 2′-methoxy substitution on the ribose moiety; m6A represents the base composition of a base-modified nucleotide, wherein the base is

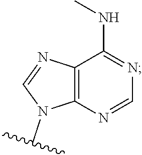 [SMILES: *n1cnc2c(NC)ncnc21]

uppercase×represents the base composition of a base-modified nucleotide, wherein the base is

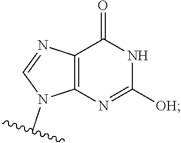 [SMILES: *n1cnc2c(=O)[nH]c(O)nc21]

uppercase B represents the base composition of a base-modified nucleotide, wherein the base is

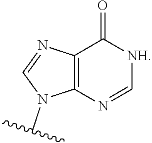 [SMILES: *n1cnc2c(=O)[nH]cnc21]

For nucleotides containing special bases as described above, unless otherwise specified, they all bear a 2′-methoxy substitution on the ribose moiety.

In the present disclosure, unless otherwise specified, the term “complementary” refers to the ability of a first oligonucleotide sequence to hybridize with a second oligonucleotide sequence and form a duplex structure under certain conditions. “At least partially complementary” means that the two sequences may be fully complementary or have no more than 5, 4, 3, or 2 mismatched base pairs in total while retaining the ability to hybridize under relevant conditions. Additionally, when two oligonucleotides are designed to hybridize with one or more single-stranded overhangs, such overhangs should not be considered mismatches for the purpose of determining complementarity. In the present disclosure, “complementary” sequences may also include or entirely consist of non-Watson-Crick base pairs and/or base pairs formed from non-natural and modified nucleotides for the purpose of meeting the above hybridization requirements. Such non-Watson-Crick base pairs include, but are not limited to, G:U wobble base pairs or Hoogsteen base pairs. Correspondingly, in the present disclosure, unless otherwise specified, “mismatch” refers to a situation in an siRNA duplex molecule where the bases at corresponding positions are not paired in a complementary manner.

In the present disclosure, unless otherwise specified, “nucleotide sequence variation” refers to a change in the type of nucleobase (A, U, G, C) at the same or a corresponding position compared to the original nucleotide sequence. For example, if a nucleobase in the original sequence is A, and the nucleobase at the same or a corresponding position is changed to U, C, or G, or the nucleotide is dT, dC, dG, etc., it is considered that there is a nucleotide sequence variation at this position. It should be noted herein that if, compared to the original nucleotide sequence, the nucleotide at the same or a corresponding position differs only in the presence or absence of modification or the type of modification, it is not considered that there is a nucleotide sequence variation at this position. For example, if a nucleobase in the original sequence is U, and the nucleotide at the same or a corresponding position is dT or another base-modified nucleotide (e.g., I, m6A, X, B), it is not considered that there is a nucleotide sequence variation at this position.

The term “sense strand” refers to the strand of an RNA molecule that carries a nucleotide sequence encoding the amino acid information of a protein, also known as the coding strand, plus strand, or positive strand, and the other nucleotide sequence that is complementary thereto is the antisense strand.

The term “antisense strand” refers to a strand that is substantially or essentially reverse complementary to a nucleotide sequence of the mRNA expressed by the target gene, wherein the nucleotide sequence is of the same length as the antisense strand.

In the present disclosure, unless otherwise specified, the term “pharmaceutically acceptable” means that carriers, vehicles, diluents, excipients, and/or salts/esters/hydrates formed therefrom are generally chemically or physically compatible with other ingredients comprising a pharmaceutical dosage form and are physiologically compatible with the receptor.

In the present disclosure, unless otherwise specified, the term “inhibition” refers to the down-regulation of the expression of a target gene due to siRNA-mediated degradation of the mRNA of the target gene. The “down-regulation” refers to a decrease in the target gene expression by 5%, 10%, 15%, 20%, 25%, 30%, 35%, 40%, 45%, 50%, 55%, 60%, 65%, 70%, 75%, 80%, 85%, 90%, 95%, or 99% or more, or even 100%, compared to the absence of siRNA treatment. A 100% decrease in the target gene expression indicates undetectable levels of the target gene expression.

In some embodiments, the siRNA may further comprise modified nucleotides as needed, and the modified nucleotides do not significantly impair or abolish the function of the siRNA to inhibit ANGPTL4 gene expression. Currently, various methods are available in the art for siRNA modification, including, for example, backbone modification (e.g., phosphate group modification), ribose group modification, and base modification (Watts, J. K., G. F. Deleavey, and M. J. Damha, Chemically modified siRNA: tools and applications. Drug Discov Today, 2008. 13(19-20): p. 842-55).

In some embodiments, at least one nucleotide in the sense strand or the antisense strand of the siRNA is a modified nucleotide. For example, the modified nucleotide is a nucleotide group in which the ribose group and optionally the phosphate group are modified, but is not limited thereto.

In some embodiments, all of the nucleotides in the sense strand and/or the antisense strand are modified nucleotides or nucleotide analogs.

In some embodiments, the modified nucleotide is selected from a 2′-methoxy nucleotide, a 2′-fluoro nucleotide, a 2′-deoxy nucleotide, a 2′,3′-seco nucleotide analog, a 2′-fluoroarabino nucleotide, a 2′-methoxyethyl nucleotide, a 2′-amino-modified nucleotide, a 2′-alkyl-modified nucleotide, a 3′-methoxy nucleotide, a 2′-allyl-modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol-modified nucleotide, an abasic nucleotide, a morpholino nucleotide, a threose nucleotide (TNA), a locked nucleotide (LNA), an unlocked nucleotide (UNA), a glycerol nucleotide (GNA), or a base-modified nucleotide, but the present disclosure is not limited thereto.

In some embodiments, the antisense strand of the siRNA is 23 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 21 nucleotides in length, wherein positions 7, 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 8, 9, 14, and 16 from the 5′ end of the antisense strand are 2′-fluoro nucleotides, and the remaining positions are 2′-methoxy nucleotides; the sense strand of the siRNA is 21 nucleotides in length, wherein positions 7, 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 21 nucleotides in length, wherein positions 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 8, 9, 10, 14, and 16 from the 5′ end of the antisense strand are 2′-fluoro nucleotides, and the remaining positions are 2′-methoxy nucleotides; the sense strand of the siRNA is 21 nucleotides in length, wherein positions 7, 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 8, 9, 10, 14, and 16 from the 5′ end of the antisense strand are 2′-fluoro nucleotides, and the remaining positions are 2′-methoxy nucleotides; the sense strand of the siRNA is 21 nucleotides in length, wherein positions 7, 9, 10, 11, and 15 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 10, 14, and 16 from the 5′ end of the antisense strand are 2′-fluoro nucleotides, and the remaining positions are 2′-methoxy nucleotides; the sense strand of the siRNA is 21 nucleotides in length, wherein positions 3, 7, 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 21 nucleotides in length, wherein positions 7, 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 3, 4, 6, 8, 10, 14, 16, 18, 20, and 22 from the 5′ end of the antisense strand are 2′-fluoro nucleotides, and the remaining positions are 2′-methoxy nucleotides; the sense strand of the siRNA is 21 nucleotides in length, wherein position 2 from the 5′ end of the sense strand is a 2′-fluoro nucleotide, and the remaining positions are 2′-methoxy nucleotides.

In some embodiments, the antisense strand of the siRNA is 23 nucleotides in length, wherein positions 2, 4, 5, 6, 8, 10, 14, 16, 18, 20, and 22 from the 5′ end of the antisense strand are 2′-fluoro nucleotides, and the remaining positions are 2′-methoxy nucleotides; the sense strand of the siRNA is 21 nucleotides in length, wherein position 2 from the 5′ end of the sense strand is a 2′-fluoro nucleotide, and the remaining positions are 2′-methoxy nucleotides.

In some embodiments, the antisense strand of the siRNA is 23 nucleotides in length, wherein positions 2, 4, 6, 7, 8, 10, 14, 16, 18, 20, and 22 from the 5′ end of the antisense strand are 2′-fluoro nucleotides, and the remaining positions are 2′-methoxy nucleotides; the sense strand of the siRNA is 21 nucleotides in length, wherein position 2 from the 5′ end of the sense strand is a 2′-fluoro nucleotide, and the remaining positions are 2′-methoxy nucleotides.

In some embodiments, the antisense strand of the siRNA is 23 nucleotides in length, wherein positions 2, 4, 6, 8, 10, 14, 16, 18, and 20 from the 5′ end of the antisense strand are 2′-fluoro nucleotides, and the remaining positions are 2′-methoxy nucleotides; the sense strand of the siRNA is 21 nucleotides in length, wherein positions 1, 3, 5, 7, 9, 10, 11, 13, 15, 17, 19, and 21 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 4, 6, 8, 10, 14, 16, 18, 20, and 22 from the 5′ end of the antisense strand are 2′-fluoro nucleotides, and the remaining positions are 2′-methoxy nucleotides; the sense strand of the siRNA is 21 nucleotides in length, wherein positions 1, 3, 5, 7, 9, 10, 11, 13, 15, 17, 19, and 21 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 8, 9, 14, and 16 from the 5′ end of the antisense strand are 2′-fluoro nucleotides, position 7 is a glycerol nucleotide (GNA), and the remaining positions are 2′-methoxy nucleotides; the sense strand of the siRNA is 21 nucleotides in length, wherein positions 7, 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 8, 9, 10, 14, and 16 from the 5′ end of the antisense strand are 2′-fluoro nucleotides, position 6 is a glycerol nucleotide (GNA), and the remaining positions are 2′-methoxy nucleotides; the sense strand of the siRNA is 21 nucleotides in length, wherein positions 7, 9, 10, 11, and 15 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 8, 9, 10, 14, and 16 from the 5′ end of the antisense strand are 2′-fluoro nucleotides, position 4 is a glycerol nucleotide (GNA), and the remaining positions are 2′-methoxy nucleotides; the sense strand of the siRNA is 21 nucleotides in length, wherein positions 7, 9, 10, 11, and 15 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 8, 9, 10, 14, and 16 from the 5′ end of the antisense strand are 2′-fluoro nucleotides, position 5 is a glycerol nucleotide (GNA), and the remaining positions are 2′-methoxy nucleotides; the sense strand of the siRNA is 21 nucleotides in length, wherein positions 7, 9, 10, 11, and 15 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 8, 9, 10, 14, and 16 from the 5′ end of the antisense strand are 2′-fluoro nucleotides, position 7 is a glycerol nucleotide (GNA), and the remaining positions are 2′-methoxy nucleotides; the sense strand of the siRNA is 21 nucleotides in length, wherein positions 7, 9, 10, 11, and 15 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 8, 9, 10, 14, and 16 from the 5′ end of the antisense strand are 2′-fluoro nucleotides, position 6 is a glycerol nucleotide (GNA), and the remaining positions are 2′-methoxy nucleotides; the sense strand of the siRNA is 21 nucleotides in length, wherein positions 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 5, 7, and 12 from the 5′ end of the antisense strand are 2′-deoxy nucleotides, position 14 is a 2′-fluoro nucleotide, and the remaining positions are 2′-methoxy nucleotides. In some embodiments, the sense strand of the siRNA is 21 nucleotides in length, wherein positions 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 5, 7, and 12 from the 5′ end of the antisense strand are 2′-deoxy nucleotides, positions 6, 8, 9, 10, 14, and 16 are 2′-fluoro nucleotides, and the remaining positions are 2′-methoxy nucleotides. In some embodiments, the sense strand of the siRNA is 21 nucleotides in length, wherein positions 7, 9, 10, and 11 from the 5′ end of the sense strand are 2′-fluoro nucleotides, and the remaining positions are 2′-methoxy nucleotides.

In some embodiments, the antisense strand of the siRNA is 21 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 21 nucleotides in length, wherein positions 5, 7, and 9 from the 5′ end of the sense strand are 2′-fluoro nucleotides, and the remaining positions are 2′-methoxy nucleotides.

In some embodiments, the antisense strand of the siRNA is 21 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 21 nucleotides in length, wherein positions 7, 9, 10, and 11 from the 5′ end of the sense strand are 2′-fluoro nucleotides, and the remaining positions are 2′-methoxy nucleotides.

In some embodiments, the antisense strand of the siRNA is 21 nucleotides in length, wherein positions 2, 4, 6, 8, 10, 12, 14, 16, 18, and 20 from the 5′ end of the antisense strand are 2′-fluoro nucleotides, and the remaining positions are 2′-methoxy nucleotides; the sense strand of the siRNA is 21 nucleotides in length, wherein positions 9, 10, and 11 from the 5′ end of the sense strand are 2′-fluoro nucleotides, and the remaining positions are 2′-methoxy nucleotides.

In some embodiments, the antisense strand of the siRNA is 21 nucleotides in length, wherein positions 2, 4, 6, 8, 10, 12, 14, 16, 18, and 20 from the 5′ end of the antisense strand are 2′-fluoro nucleotides, and the remaining positions are 2′-methoxy nucleotides; the sense strand of the siRNA is 21 nucleotides in length, wherein positions 1, 3, 5, 7, 9, 11, 13, 15, 17, 19, and 21 from the 5′ end of the sense strand are 2′-fluoro nucleotides, and the remaining positions are 2′-methoxy nucleotides.

In some embodiments, the antisense strand of the siRNA is 21 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 19 nucleotides in length, wherein positions 7, 8, and 9 from the 5′ end of the sense strand are 2′-fluoro nucleotides, and the remaining positions are 2′-methoxy nucleotides.

In some embodiments, the antisense strand of the siRNA is 19 nucleotides in length, wherein positions 2, 4, 6, 8, 10, 12, 14, 16, and 18 from the 5′ end of the antisense strand are 2′-fluoro nucleotides, and the remaining positions are 2′-methoxy nucleotides; the sense strand of the siRNA is 19 nucleotides in length, wherein positions 7, 8, and 9 from the 5′ end of the sense strand are 2′-fluoro nucleotides, and the remaining positions are 2′-methoxy nucleotides.

In some embodiments, the antisense strand of the siRNA is 23 nucleotides in length, wherein position 14 from the 5′ end of the antisense strand is a 2′-fluoro nucleotide, positions 2, 5, and 7 are 2′-deoxy nucleotides, position 12 is a threose nucleotide, and the remaining positions are 2′-methoxy nucleotides; the sense strand of the siRNA is 21 nucleotides in length, wherein positions 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 21 nucleotides in length, wherein positions 9, 10, and 11 from the 5′ end of the sense strand are 2′-fluoro nucleotides, position 1 is a threose nucleotide, and the remaining positions are 2′-methoxy nucleotides.

In some embodiments, the antisense strand of the siRNA is 23 nucleotides in length, wherein positions 2, 6, 14, and 16 from the 5′ end of the antisense strand are 2′-fluoro nucleotides, position 22 is a threose nucleotide, and the remaining positions are 2′-methoxy nucleotides; the sense strand of the siRNA is 21 nucleotides in length, wherein positions 9, 10, and 11 from the 5′ end of the sense strand are 2′-fluoro nucleotides, and the remaining positions are 2′-methoxy nucleotides.

In some embodiments, the antisense strand of the siRNA is 23 nucleotides in length, wherein positions 2, 6, 14, and 16 from the 5′ end of the antisense strand are 2′-fluoro nucleotides, position 23 is a threose nucleotide, and the remaining positions are 2′-methoxy nucleotides; the sense strand of the siRNA is 21 nucleotides in length, wherein positions 9, 10, and 11 from the 5′ end of the sense strand are 2′-fluoro nucleotides, and the remaining positions are 2′-methoxy nucleotides.

In some embodiments, the antisense strand of the siRNA is 22 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 20 nucleotides in length, wherein positions 8, 9, and 10 from the 5′ end of the sense strand are 2′-fluoro nucleotides, position 1 is a threose nucleotide, and the remaining positions are 2′-methoxy nucleotides.

In some embodiments, the antisense strand of the siRNA is 22 nucleotides in length, wherein positions 2, 6, 14, and 16 from the 5′ end of the antisense strand are 2′-fluoro nucleotides, position 21 is a threose nucleotide, and the remaining positions are 2′-methoxy nucleotides; the sense strand of the siRNA is 20 nucleotides in length, wherein positions 8, 9, and 10 from the 5′ end of the sense strand are 2′-fluoro nucleotides, and the remaining positions are 2′-methoxy nucleotides.

In some embodiments, the antisense strand of the siRNA is 22 nucleotides in length, wherein positions 2, 6, 14, and 16 from the 5′ end of the antisense strand are 2′-fluoro nucleotides, position 22 is a threose nucleotide, and the remaining positions are 2′-methoxy nucleotides; the sense strand of the siRNA is 20 nucleotides in length, wherein positions 8, 9, and 10 from the 5′ end of the sense strand are 2′-fluoro nucleotides, and the remaining positions are 2′-methoxy nucleotides.

In some embodiments, the antisense strand of the siRNA is 22 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 20 nucleotides in length, wherein positions 8, 9, and 10 from the 5′ end of the sense strand are 2′-fluoro nucleotides, and the remaining positions are 2′-methoxy nucleotides.

In some embodiments, the antisense strand of the siRNA is 22 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 20 nucleotides in length, wherein positions 6, 8, 9, and 10 from the 5′ end of the sense strand are 2′-fluoro nucleotides, and the remaining positions are 2′-methoxy nucleotides.

In some embodiments, the antisense strand of the siRNA is 22 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 20 nucleotides in length, wherein positions 8, 9, and 10 from the 5′ end of the sense strand are 2′-fluoro nucleotides, position 6 is a 2′-deoxy nucleotide, and the remaining positions are 2′-methoxy nucleotides.

In some embodiments, the antisense strand of the siRNA is 22 nucleotides in length, wherein positions 2, 6, 8, 9, 14, and 16 from the 5′ end of the antisense strand are 2′-fluoro nucleotides, and the remaining positions are 2′-methoxy nucleotides; the sense strand of the siRNA is 20 nucleotides in length, wherein positions 6, 8, 9, and 10 from the 5′ end of the sense strand are 2′-fluoro nucleotides, and the remaining positions are 2′-methoxy nucleotides.

In some embodiments, the antisense strand of the siRNA is 21 nucleotides in length, wherein positions 2, 6, 14, and 16 from the 5′ end of the antisense strand are 2′-fluoro nucleotides, and the remaining positions are 2′-methoxy nucleotides; the sense strand of the siRNA is 19 nucleotides in length, wherein positions 7, 8, and 9 from the 5′ end of the sense strand are 2′-fluoro nucleotides, position 1 is a threose nucleotide, and the remaining positions are 2′-methoxy nucleotides.

In some embodiments, the antisense strand of the siRNA is 21 nucleotides in length, wherein positions 2, 6, 14, and 16 from the 5′ end of the antisense strand are 2′-fluoro nucleotides, position 20 is a threose nucleotide, and the remaining positions are 2′-methoxy nucleotides; the sense strand of the siRNA is 19 nucleotides in length, wherein positions 7, 8, and 9 from the 5′ end of the sense strand are 2′-fluoro nucleotides, and the remaining positions are 2′-methoxy nucleotides.

In some embodiments, the antisense strand of the siRNA is 21 nucleotides in length, wherein positions 2, 6, 14, and 16 from the 5′ end of the antisense strand are 2′-fluoro nucleotides, position 21 is a threose nucleotide, and the remaining positions are 2′-methoxy nucleotides; the sense strand of the siRNA is 19 nucleotides in length, wherein positions 7, 8, and 9 from the 5′ end of the sense strand are 2′-fluoro nucleotides, and the remaining positions are 2′-methoxy nucleotides.

In some embodiments, the modified nucleotide is a nucleotide in which the phosphate group is modified by a phosphorothioate group. That is, a non-bridging oxygen atom in a phosphodiester bond is substituted by a sulfur atom, thereby replacing the phosphodiester bond with a phosphorothioate bond.

In some embodiments, the 5′ and 3′ ends of the sense strand independently comprise 1 or 2 phosphorothioate linkages, respectively; and/or the 5′ and 3′ ends of the antisense strand independently comprise 1 or 2 phosphorothioate linkages, respectively.

In some embodiments, at least one of the following positions contains a phosphorothioate linkage: between nucleotides at positions 1 and 2 from the 5′ end of the sense strand, between nucleotides at positions 2 and 3 from the 5′ end of the sense strand, between nucleotides at positions 1 and 2 from the 3′ end of the sense strand, between nucleotides at positions 2 and 3 from the 3′ end of the sense strand, between nucleotides at positions 1 and 2 from the 3′ end of the antisense strand, between nucleotides at positions 2 and 3 from the 3′ end of the antisense strand, between nucleotides at positions 1 and 2 from the 5′ end of the antisense strand, and between nucleotides at positions 2 and 3 from the 5′ end of the antisense strand; preferably, at least four of these positions contain a phosphorothioate linkage; in some embodiments, at least six of these positions contain a phosphorothioate linkage; in some embodiments, all eight of these positions contain a phosphorothioate linkage.

In some embodiments, there is a phosphorothioate linkage between nucleotides at positions 1 and 2 and between nucleotides at positions 2 and 3 from the 5′ end of the sense strand.

In some embodiments, there is a phosphorothioate linkage between nucleotides at positions 1 and 2 and between nucleotides at positions 2 and 3 from the 5′ end of the sense strand, and there is a phosphorothioate linkage between nucleotides at positions 1 and 2 and between nucleotides at positions 2 and 3 from the 3′ end of the sense strand.

In some embodiments, there is a phosphorothioate linkage between nucleotides at positions 1 and 2 and between nucleotides at positions 2 and 3 from the 3′ end of the antisense strand, and there is a phosphorothioate linkage between nucleotides at positions 1 and 2 and between nucleotides at positions 2 and 3 from the 5′ end of the antisense strand.

In some embodiments, the sense strand may comprise one or more blocking residues or moieties, referred to as “capping residues”. A “capping residue” is a non-nucleotide compound or another moiety that can be incorporated at one or more ends of the nucleotide sequence of an siRNA. In some embodiments, the capping residue is present at the 5′ end, the 3′ end, or both the 5′ end and the 3′ end of the sense strand.

In some embodiments, an inverted abasic residue (iab) is added as a capping residue. See F. Czaudema, Nucleic Acids Res., 2003, 31(11), 2705-16. In some embodiments, the 5′ end and/or the 3′ end of the sense strand may comprise more than one inverted abasic deoxyribose moiety as a capping residue.

In some embodiments, one or more inverted abasic residues (iab) are added to the 3′ end of the sense strand. In some embodiments, one or more inverted abasic residues (iab) are added to the 5′ end of the sense strand. In some embodiments, one or more inverted abasic residues may be inserted between the linker-targeting ligand moiety and the nucleotide sequence of the sense strand of the siRNA. In some embodiments, one or more inverted abasic residues are included at or near one or more ends of the sense strand of the siRNA.

In some embodiments, one or more inverted abasic residues (iab) are added to the 5′ end of the sense strand. In some embodiments, one or more inverted abasic residues may be inserted between the linker-targeting ligand moiety and the nucleotide sequence of the sense strand of the siRNA.

The inverted abasic residues may be linked via phosphate, phosphorothioate, or other internucleotide linkages.

In some embodiments, the first nucleotide at the 5′ end of the antisense strand is selected from the following structures:

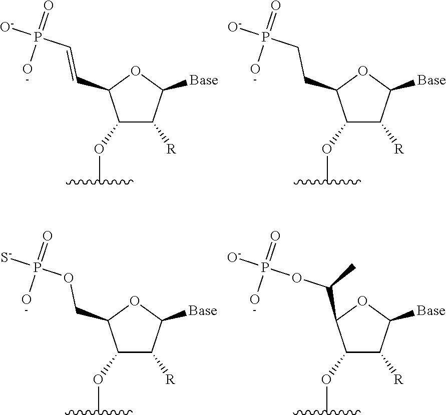

wherein Base is A, U, G, C, T, or another nucleotide base.

In some embodiments, the first nucleotide at the 5′ end of the antisense strand is selected from the following structures:

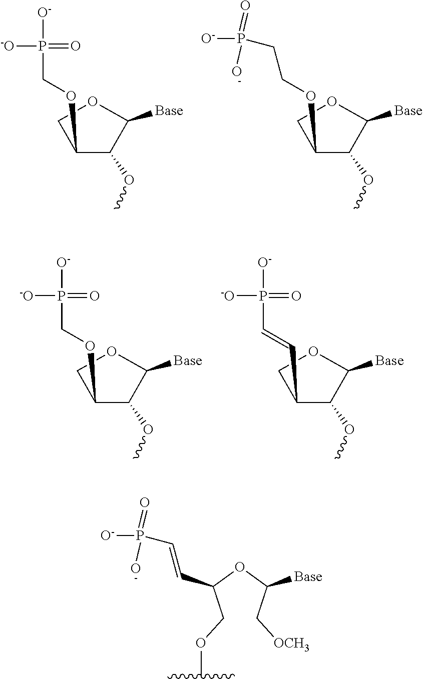

wherein Base is A, U, G, C, T, or another nucleotide base.

In some embodiments, the first nucleotide at the 5′ end of the antisense strand is an (E)-vinylphosphonate-modified nucleotide.

In some embodiments, the siRNA comprises at least one base-modified nucleotide.

In some embodiments, the base of the base-modified nucleotide is selected from the following structures:

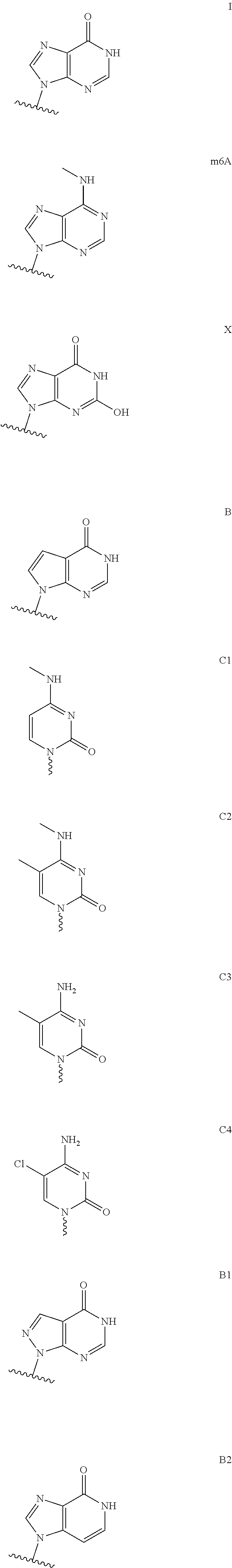

In some embodiments, the base-modified nucleotides are located at positions 5, 6, 7, and 8 of the antisense strand of the siRNA.

In some embodiments, the base-modified nucleotides are located at the overhang of the single-stranded nucleotide of the siRNA.

Preferably, the antisense strand of the siRNA contains an overhang of 2 nucleotides, and the base-modified nucleotide is the first nucleotide at the overhang of the antisense strand of the siRNA.

Preferably, the antisense strand of the siRNA contains an overhang of 2 nucleotides, and the base-modified nucleotide is the second nucleotide at the overhang of the antisense strand of the siRNA.

In the present disclosure, unless otherwise specified, a “conjugation” refers to a covalent linkage between two or more chemical moieties; a “conjugate” refers to a compound formed by covalent linkage between chemical moieties; and an “siRNA conjugate” refers to a compound formed by covalent linkage of one or more chemical moieties to an siRNA. It should be noted herein that each chemical moiety may be directly linked to the siRNA or linked to the siRNA via a linker.

In the present disclosure, unless otherwise specified, the “−” in “linker-targeting ligand” refers to a covalent linkage between the linker and the targeting ligand.

In some embodiments, the siRNA of the present disclosure may be conjugated with a pharmaceutically acceptable conjugate molecule to form an siRNA conjugate. In some embodiments, the siRNA is covalently conjugated to the conjugate molecule. To minimize the possible effect of conjugation on siRNA activity, the conjugation site between the siRNA and the conjugation molecule may be at the 3′ or 5′ end of the sense strand of the siRNA, or at the 5′ end of the antisense strand of the siRNA. In some embodiments, the conjugation site between the siRNA and the conjugation molecule may also be within the internal sequence of the siRNA.

The pharmaceutically acceptable targeting ligand may be a targeting ligand conventionally used in the field of siRNA delivery, such as, but not limited to, one or more of the following targeting ligands or derivatives thereof: lipophilic molecules, such as cholesterol, bile acids, vitamins (e.g., vitamin E), lipid molecules with varying chain lengths; polymers, such as polyethylene glycol; polypeptides, such as cell penetrating peptides; aptamers; antibodies; quantum dots; carbohydrates such as lactose, polylactose, mannose, galactose, N-acetylgalactosamine (GalNAc); folate; or ligands for receptors expressed on hepatocytes, such as asialoglycoproteins, asialoglycoprotein residues, lipoproteins (e.g., high-density lipoprotein, low-density lipoprotein), glucagon, neurotransmitters (e.g., epinephrine), growth factors, transferrin.

In some embodiments, the targeting ligand is N-acetylgalactosamine.

In some embodiments, the targeting ligand is directly linked to the 3′ end of the sense strand of the siRNA. In some embodiments, the targeting ligand is directly linked to the 5′ end of the sense strand of the siRNA. In some embodiments, the targeting ligand is linked to the 3′ end of the sense strand of the siRNA via a linker. In some embodiments, the targeting ligand is linked to the 5′ end of the sense strand of the siRNA via a linker.

In some embodiments, the linker-targeting ligand moiety has the following structure:

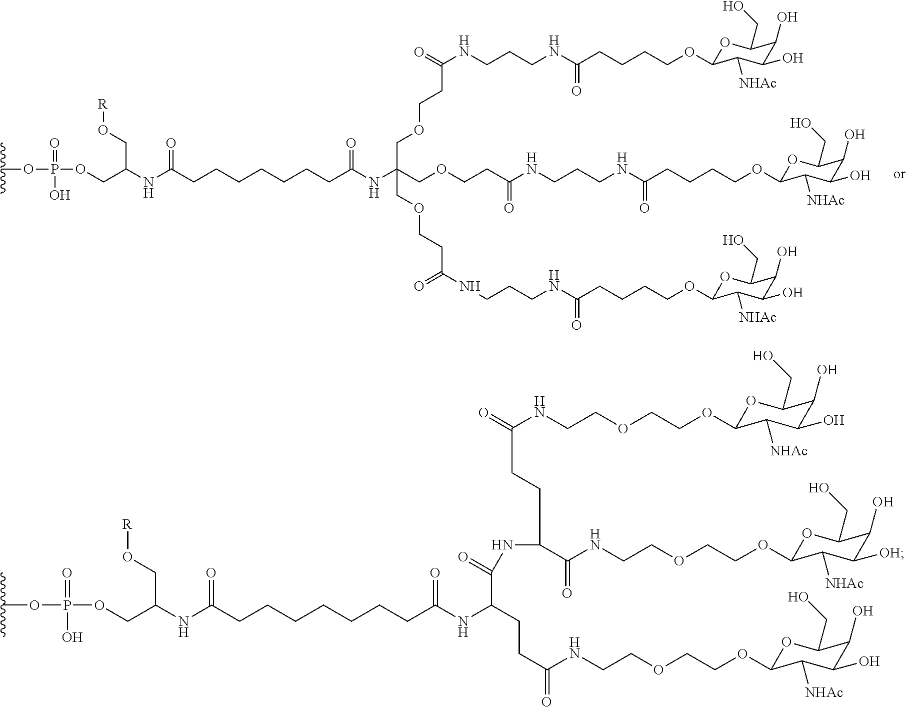

wherein R is selected from hydrogen and an auxiliary group. In some embodiments, the auxiliary groups include, but are not limited to, long-chain alkyl groups, long-chain alkenyl groups, long-chain alkynyl groups, cholesterol groups, cholesterol-like groups, polyethylene glycol groups; in some embodiments, the auxiliary groups include, but are not limited to, polypeptides.

In some embodiments, the linker-targeting ligand moiety is GalNAc (L96) having the following structure:

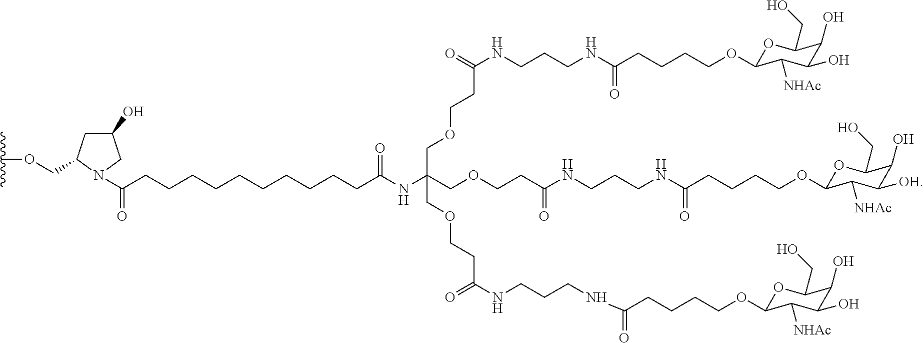 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, GalNAc (L96) is linked to the 3′ end of the sense strand of the siRNA.

In some embodiments, GalNAc (L96) is linked to an inverted abasic residue (iab) at the 3′ end of the sense strand of the siRNA.

The present disclosure further provides a pharmaceutical composition, comprising the siRNA according to the present disclosure as an active ingredient and a pharmaceutically acceptable carrier.

The term “pharmaceutically acceptable carrier” refers to a carrier for the administration of a therapeutic agent. Such carriers include, but are not limited to, saline, buffered saline, glucose, water, glycerol, ethanol, and combinations thereof. For orally administered drugs, pharmaceutically acceptable carriers include, but are not limited to, pharmaceutically acceptable excipients such as inert diluents, disintegrants, binders, lubricants, sweeteners, flavoring agents, colorants, and preservatives, as known to those skilled in the art.

Particularly, it may be contemplated that pharmaceutically acceptable carriers allow systemic administration of the siRNA or siRNA conjugate of the present disclosure. However, enteral administration, parenteral administration, and transdermal or transmucosal (e.g., insufflation, buccal, vaginal, anal) administration, as well as drug inhalation may also be contemplated as feasible methods for administering the compounds of the present disclosure to patients in need of medical intervention. When parenteral administration is employed, it may involve injection of the compounds of the present disclosure directly into or at least in close proximity to the diseased tissue. However, intravenous, intraarterial, subcutaneous, intramuscular, intraperitoneal, intradermal, intrathecal, and other administrations of the compounds of the present disclosure also fall within the technical expertise of a skilled personnel, such as an attending physician.

For intramuscular, subcutaneous, and intravenous use, the pharmaceutical composition of the present disclosure will generally be provided in a sterile aqueous solution or suspension buffered to an appropriate pH and isotonicity. In a preferred embodiment, the carrier consists solely of an aqueous buffer. In this context, “solely” means the absence of auxiliary agents or encapsulating substances that might affect or mediate the uptake of the siRNA in cells expressing the ANGPTL4 gene. Aqueous suspensions according to the present disclosure may include a suspending agent such as cellulose derivatives, sodium alginate, polyvinylpyrrolidone, and tragacanth, and a wetting agent such as lecithin. Suitable preservatives for aqueous suspensions include ethyl and n-propyl p-hydroxybenzoate. Pharmaceutical compositions useful according to the present disclosure also include encapsulated formulations to protect the siRNA from rapid clearance from the body, such as controlled-release formulations, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparing such formulations will be apparent to those skilled in the art. Liposomal suspensions may also be used as pharmaceutically acceptable carriers. These carriers can be prepared according to methods known to those skilled in the art, such as those described in PCT publication WO 91/06309, which is incorporated herein by reference.

In some embodiments of the present disclosure, the pharmaceutical composition comprises one siRNA according to the present disclosure. In some other embodiments of the present disclosure, the pharmaceutical composition comprises at least two siRNAs according to the present disclosure (e.g., but not limited to, 2, 3, 4, 5, 6, 7, 8, 9, 10, or more) as active ingredients. Preferably, the at least two siRNAs according to the present disclosure each target a different target sequence in the ANGPTL4 gene, thereby enabling simultaneous action against different target sequences to produce a synergistic effect. In this context, the term “different target sequences” means that the target sequences do not overlap, or that the number of overlapping continuous nucleotides between target sequences is less than 5 (e.g., 4, 3, 2, 1, or 0 overlapping continuous nucleotides). In this context, the at least two siRNAs according to the present disclosure may be present in any different ratios. Preferably, the at least two siRNAs according to the present disclosure may be present in a molar ratio of 1:100 to 100:1 relative to each other; more preferably, the at least two siRNAs according to the present disclosure may be present in a molar ratio of 1:10 to 10:1, 1:5 to 5:1, or 1:2 to 2:1 relative to each other. In some embodiments of the present disclosure, the at least two siRNAs according to the present disclosure are present in equal molar ratios.

### DETAILED DESCRIPTION OF THE PREFERRED EMBODIMENT

Those skilled in the art are aware that the siRNAs according to the present disclosure can be obtained by conventional siRNA preparation methods in the art (e.g., solid-phase synthesis and liquid-phase synthesis), wherein both solid-phase synthesis and liquid-phase synthesis are commercially available as custom services. Those skilled in the art are also fully aware that modified nucleotide groups can be introduced into the siRNAs according to the present disclosure by using nucleotide monomers with corresponding modifications. Methods for preparing nucleotide monomers with corresponding modifications are well known to those skilled in the art, and commercial monomers are also available on the market.

### Example 1: SiRNA Synthesis

For the sense and antisense strands of the siRNA sequences of the present disclosure as well as the sense and antisense strands of the modified duplexes, deoxynucleoside CPG was used as a solid-phase support. The sense strands were synthesized using the solid-phase support, while the antisense strands were synthesized using universal CPG.

A 48-channel synthesizer was used for sequence synthesis at a 0.2 μmol scale. Phosphoramidite monomers were used at a concentration of 0.05 M, with 0.3 M BTT as the activator.

Cleavage and deprotection were performed on sequences in a 1.5 mL tube, using AMA in the first step and triethylamine trihydrofluoride for 2′-deprotection in the second step. For sequences containing full 2′-modifications, hydrolysis with ammonia was required. The cleaved and deprotected sequences were precipitated using a mixture of acetone and ethanol (80:20) and dissolved in RNase-free water. The sequences were analyzed by LC-MS to determine accuracy, quantified by spectrophotometry, and assessed for purity by HPLC.

After HPLC purification, lyophilization, and quality control, the sequences were subjected to sodium acetate/alcohol precipitation for salt exchange. Desalting was performed using a 3 KD ultrafiltration tube, followed by spectrophotometric quantification of sense and antisense strands, which were mixed in a 1:1 ratio and annealed to form an siRNA duplex.

TABLE 1
Sense and antisense strand sequences of unmodified siRNA duplexes

| Duplex | 5′-sense-3′ | SEQ ID | 5′-antisense-3′ | SEQ ID |
|---|---|---|---|---|
| No. | (sense strand sequence) | NO | (antisense strand sequence) | NO |
| 1 | CCGUACCCUUCUCCACUUGGA | 212 | UCCAAGUGGAGAAGGGUACGGAG | 1 |
| 2 | CGUACCCUUCUCCACUUGGGA | 213 | UCCCAAGUGGAGAAGGGUACGGA | 2 |
| 3 | CUCUGGAGGCUGGUGGUUUGA | 214 | UCAAACCACCAGCCUCCAGAGAG | 3 |
| 4 | AGGCCACCACCAUGUUGAUCU | 215 | AGAUCAACAUGGUGGUGGCCUGC | 4 |
| 5 | ACUUGGGACCAGGAUCACGAU | 216 | AUCGUGAUCCUGGUCCCAAGUGG | 5 |
| 6 | UGAGGUCCUUCACAGCCUGCA | 217 | UGCAGGCUGUGAAGGACCUCAGG | 6 |
| 7 | CACUUGGGACCAGGAUCACGA | 218 | UCGUGAUCCUGGUCCCAAGUGGA | 7 |
| 8 | ACCCUUCUCCACUUGGGACCA | 219 | UGGUCCCAAGUGGAGAAGGGUAC | 8 |
| 9 | CCACUUGGGACCAGGAUCACA | 220 | UGUGAUCCUGGUCCCAAGUGGAG | 9 |
| 10 | GGCCACCACCAUGUUGAUCCA | 221 | UGGAUCAACAUGGUGGUGGCCUG | 10 |
| 11 | GGACCCUGAGGUCCUUCACAA | 222 | UUGUGAAGGACCUCAGGGUCCAC | 11 |
| 12 | AUCUUCUGGAAGACCUGGCGA | 223 | UCGCCAGGUCUUCCAGAAGAUUC | 12 |
| 13 | GUACUUCCGCUCCAUCCCACA | 224 | UGUGGGAUGGAGCGGAAGUACUG | 13 |
| 14 | AGGGAUUGCCAGGAGCUGUUU | 225 | AAACAGCUCCUGGCAAUCCCUGG | 14 |
| 15 | CCACCACCAUGUUGAUCCAGU | 226 | ACUGGAUCAACAUGGUGGUGGCC | 15 |
| 16 | GGGACCAGGAUCACGACCUCU | 227 | AGAGGUCGUGAUCCUGGUCCCAA | 16 |
| 17 | CUUCUCCACUUGGGACCAGGA | 228 | UCCUGGUCCCAAGUGGAGAAGGG | 17 |
| 18 | GCCACCACCAUGUUGAUCCAA | 229 | UUGGAUCAACAUGGUGGUGGCCU | 18 |
| 19 | UCUCCACUUGGGACCAGGAUU | 230 | AAUCCUGGUCCCAAGUGGAGAAG | 19 |
| 20 | GGCCAGUACUUCCGCUCCAUU | 231 | AAUGGAGCGGAAGUACUGGCCGU | 20 |
| 21 | ACUGCGCCAAGAGCCUCUCUA | 232 | UAGAGAGGCUCUUGGCGCAGUUC | 21 |
| 22 | CUUCCACAAGGUGGCCCAGCA | 233 | UGCUGGGCCACCUUGUGGAAGAG | 22 |
| 23 | ACAGCCUGCAGACACAACUCA | 234 | UGAGUUGUGUCUGCAGGCUGUGA | 23 |
| 24 | CCUGAGGUCCUUCACAGCCUA | 235 | UAGGCUGUGAAGGACCUCAGGGU | 24 |
| 25 | AGCAACUCUUCCACAAGGUGA | 236 | UCACCUUGUGGAAGAGUUGCUGG | 25 |
| 26 | CCCUUCUCCACUUGGGACCAA | 237 | UUGGUCCCAAGUGGAGAAGGGUA | 26 |
| 27 | ACGACCUCCGCAGGGACAAGA | 238 | UCUUGUCCCUGCGGAGGUCGUGA | 27 |
| 28 | ACUCUUCCACAAGGUGGCCCA | 239 | UGGGCCACCUUGUGGAAGAGUUG | 28 |
| 29 | GCAGACACAACUCAAGGCUCA | 240 | UGAGCCUUGAGUUGUGUCUGCAG | 29 |
| 30 | UCCACUUGGGACCAGGAUCAU | 241 | AUGAUCCUGGUCCCAAGUGGAGA | 30 |
| 31 | CACGACCUCCGCAGGGACAAA | 242 | UUUGUCCCUGCGGAGGUCGUGAU | 31 |
| 32 | CAGGCCACCACCAUGUUGAUU | 243 | AAUCAACAUGGUGGUGGCCUGCA | 32 |
| 33 | AGAAGCAGCACCUGCGAAUUU | 244 | AAAUUCGCAGGUGCUGCUUCUCC | 33 |
| 34 | AGCAGCACCUGCGAAUUCAGU | 245 | ACUGAAUUCGCAGGUGCUGCUUC | 34 |
| 35 | GAAGCAGCACCUGCGAAUUCA | 246 | UGAAUUCGCAGGUGCUGCUUCUC | 35 |
| 36 | ACCACCAUGUUGAUCCAGCCU | 247 | AGGCUGGAUCAACAUGGUGGUGG | 36 |
| 37 | GGAUCCAGCAACUCUUCCACA | 248 | UGUGGAAGAGUUGCUGGAUCCUG | 37 |
| 38 | CCAGCAACUCUUCCACAAGGU | 249 | ACCUUGUGGAAGAGUUGCUGGAU | 38 |
| 39 | CAGACACAACUCAAGGCUCAA | 250 | UUGAGCCUUGAGUUGUGUCUGCA | 39 |
| 40 | CCUCUCUGGAGGCUGGUGGUU | 251 | AACCACCAGCCUCCAGAGAGGCU | 40 |
| 41 | UGCAGGCCACCACCAUGUUGA | 252 | UCAACAUGGUGGUGGCCUGCAGC | 41 |
| 42 | CCACCAUGUUGAUCCAGCCCA | 253 | UGGGCUGGAUCAACAUGGUGGUG | 42 |
| 43 | GCAGCACCUGCGAAUUCAGCA | 254 | UGCUGAAUUCGCAGGUGCUGCUU | 43 |
| 44 | AGAACUGCGCCAAGAGCCUCU | 255 | AGAGGCUCUUGGCGCAGUUCUUG | 44 |
| 45 | CCUGCAGCCAUUCCAACCUCA | 256 | UGAGGUUGGAAUGGCUGCAGGUG | 45 |
| 46 | GCAGGCCACCACCAUGUUGAU | 257 | AUCAACAUGGUGGUGGCCUGCAG | 46 |
| 47 | CAACUCAAGGCUCAGAACAGU | 258 | ACUGUUCUGAGCCUUGAGUUGUG | 47 |
| 48 | CUUCCGCUCCAUCCCACAGCA | 259 | UGCUGUGGGAUGGAGCGGAAGUA | 48 |
| 49 | CUCAGAACAGCAGGAUCCAGU | 260 | ACUGGAUCCUGCUGUUCUGAGCC | 49 |
| 50 | CUCUUCCACAAGGUGGCCCAA | 261 | UUGGGCCACCUUGUGGAAGAGUU | 50 |
| 51 | AGAGCCUCUCUGGAGGCUGGU | 262 | ACCAGCCUCCAGAGAGGCUCUUG | 51 |
| 52 | GAGCCUCUCUGGAGGCUGGUA | 263 | UACCAGCCUCCAGAGAGGCUCUU | 52 |
| 53 | GCUCAGAACAGCAGGAUCCAA | 264 | UUGGAUCCUGCUGUUCUGAGCCU | 53 |
| 54 | GAGGCUGGUGGUUUGGCACCU | 265 | AGGUGCCAAACCACCAGCCUCCA | 54 |
| 55 | GAACUGCGCCAAGAGCCUCUU | 266 | AAGAGGCUCUUGGCGCAGUUCUU | 55 |
| 56 | AAGAACUGCGCCAAGAGCCUU | 267 | AAGGCUCUUGGCGCAGUUCUUGU | 56 |
| 57 | CUUCACAGCCUGCAGACACAA | 268 | UUGUGUCUGCAGGCUGUGAAGGA | 57 |
| 58 | UCACGACCUCCGCAGGGACAA | 269 | UUGUCCCUGCGGAGGUCGUGAUC | 58 |
| 59 | AGACACAACUCAAGGCUCAGA | 270 | UCUGAGCCUUGAGUUGUGUCUGC | 59 |
| 60 | AGAACAGCAGGAUCCAGCAAU | 271 | AUUGCUGGAUCCUGCUGUUCUGA | 60 |
| 61 | GUGGUUUGGCACCUGCAGCCA | 272 | UGGCUGCAGGUGCCAAACCACCA | 61 |
| 62 | CCACAAGGUGGCCCAGCAGCA | 273 | UGCUGCUGGGCCACCUUGUGGAA | 62 |
| 63 | CCUCCGCAGGGACAAGAACUA | 274 | UAGUUCUUGUCCCUGCGGAGGUC | 63 |
| 64 | CGACCUCCGCAGGGACAAGAA | 275 | UUCUUGUCCCUGCGGAGGUCGUG | 64 |
| 65 | ACAACUCAAGGCUCAGAACAA | 276 | UUGUUCUGAGCCUUGAGUUGUGU | 65 |
| 66 | CAAGAACUGCGCCAAGAGCCU | 277 | AGGCUCUUGGCGCAGUUCUUGUC | 66 |
| 67 | ACACAACUCAAGGCUCAGAAU | 278 | AUUCUGAGCCUUGAGUUGUGUCU | 67 |
| 68 | GCUGGUGGUUUGGCACCUGCA | 279 | UGCAGGUGCCAAACCACCAGCCU | 68 |
| 69 | GACCUCCGCAGGGACAAGAAU | 280 | AUUCUUGUCCCUGCGGAGGUCGU | 69 |
| 70 | CUCCGCAGGGACAAGAACUGU | 281 | ACAGUUCUUGUCCCUGCGGAGGU | 70 |
| 71 | CAAGGCUCAGAACAGCAGGAU | 282 | AUCCUGCUGUUCUGAGCCUUGAG | 71 |
| 72 | AAGCCUGCCCGAAGAAAGAGA | 283 | UCUCUUUCUUCGGGCAGGCUUGG | 72 |
| 73 | CAAGCCUGCCCGAAGAAAGAA | 284 | UUCUUUCUUCGGGCAGGCUUGGC | 73 |
| 74 | GACACAACUCAAGGCUCAGAA | 285 | UUCUGAGCCUUGAGUUGUGUCUG | 74 |
| 75 | AGCCUGCCCGAAGAAAGAGGU | 286 | ACCUCUUUCUUCGGGCAGGCUUG | 75 |
| 76 | ACUCAAGGCUCAGAACAGCAA | 287 | UUGCUGUUCUGAGCCUUGAGUUG | 76 |
| 77 | GGCCAAGCCUGCCCGAAGAAA | 288 | UUUCUUCGGGCAGGCUUGGCCAC | 77 |
| 78 | ACAGCAGGAUCCAGCAACUCU | 289 | AGAGUUGCUGGAUCCUGCUGUUC | 78 |
| 79 | GCCAAGCCUGCCCGAAGAAAA | 290 | UUUUCUUCGGGCAGGCUUGGCCA | 79 |
| 80 | CCAAGCCUGCCCGAAGAAAGA | 291 | UCUUUCUUCGGGCAGGCUUGGCC | 80 |
| 81 | UCAAGGCUCAGAACAGCAGGA | 292 | UCCUGCUGUUCUGAGCCUUGAGU | 81 |
| 82 | GGACAAGAACUGCGCCAAGAA | 293 | UUCUUGGCGCAGUUCUUGUCCCU | 82 |
| 83 | CUCAAGGCUCAGAACAGCAGA | 294 | UCUGCUGUUCUGAGCCUUGAGUU | 83 |
| 84 | AACAGCAGGAUCCAGCAACUU | 295 | AAGUUGCUGGAUCCUGCUGUUCU | 84 |
| 85 | GCCUGCCCGAAGAAAGAGGCU | 296 | AGCCUCUUUCUUCGGGCAGGCUU | 85 |
| 86 | AAGACGGUGACUCUUGGCUCU | 297 | AGAGCCAAGAGUCACCGUCUUUC | 86 |
| 87 | AGACGGUGACUCUUGGCUCUA | 298 | UAGAGCCAAGAGUCACCGUCUUU | 87 |
| 88 | UUGGCUCUGCCCGAGGAUGUA | 299 | UACAUCCUCGGGCAGAGCCAAGA | 88 |
| 89 | UGGCUCUGCCCGAGGAUGUGA | 300 | UCACAUCCUCGGGCAGAGCCAAG | 89 |
| 90 | CCAAGGAGGGGCCAUCUGGAA | 301 | UUCCAGAUGGCCCCUCCUUGGAG | 90 |
| 91 | AGGAGGGGCCAUCUGGAAACU | 302 | AGUUUCCAGAUGGCCCCUCCUUG | 91 |
| 92 | GGAGGGGCCAUCUGGAAACUU | 303 | AAGUUUCCAGAUGGCCCCUCCUU | 92 |
| 93 | GAGGGGCCAUCUGGAAACUUA | 304 | UAAGUUUCCAGAUGGCCCCUCCU | 93 |
| 94 | GAAACUUGUGGACAGAGAAGA | 305 | UCUUCUCUGUCCACAAGUUUCCA | 94 |
| 95 | UGUGGACAGAGAAGAAGACCA | 306 | UGGUCUUCUUCUCUGUCCACAAG | 95 |
| 96 | GAAGAAGACCACGACUGGAGA | 307 | UCUCCAGUCGUGGUCUUCUUCUC | 96 |
| 97 | AAGAAGACCACGACUGGAGAA | 308 | UUCUCCAGUCGUGGUCUUCUUCU | 97 |
| 98 | AUGCGUUGCCUCCUGAGAUCA | 309 | UGAUCUCAGGAGGCAACGCAUGC | 98 |
| 99 | CCUCCUGAGAUCGAGGCUGCA | 310 | UGCAGCCUCGAUCUCAGGAGGCA | 99 |
| 100 | CUCCUGAGAUCGAGGCUGCAA | 311 | UUGCAGCCUCGAUCUCAGGAGGC | 100 |
| 101 | CCUGAGAUCGAGGCUGCAGGA | 312 | UCCUGCAGCCUCGAUCUCAGGAG | 101 |
| 102 | CUGAGAUCGAGGCUGCAGGAU | 313 | AUCCUGCAGCCUCGAUCUCAGGA | 102 |
| 103 | UGAGAUCGAGGCUGCAGGAUA | 314 | UAUCCUGCAGCCUCGAUCUCAGG | 103 |
| 104 | AGGCGUGGACCAAGGGGCAUA | 315 | UAUGCCCCUUGGUCCACGCCUCU | 104 |
| 105 | GGACCAAGGGGCAUGGAGCUU | 316 | AAGCUCCAUGCCCCUUGGUCCAC | 105 |
| 106 | GACCAAGGGGCAUGGAGCUUU | 317 | AAAGCUCCAUGCCCCUUGGUCCA | 106 |
| 107 | ACCAAGGGGCAUGGAGCUUCA | 318 | UGAAGCUCCAUGCCCCUUGGUCC | 107 |
| 108 | CCAAGGGGCAUGGAGCUUCAU | 319 | AUGAAGCUCCAUGCCCCUUGGUC | 108 |
| 109 | CAAGGGGCAUGGAGCUUCACU | 320 | AGUGAAGCUCCAUGCCCCUUGGU | 109 |
| 110 | AAGGGGCAUGGAGCUUCACUU | 321 | AAGUGAAGCUCCAUGCCCCUUGG | 110 |
| 111 | AGGGGCAUGGAGCUUCACUCU | 322 | AGAGUGAAGCUCCAUGCCCCUUG | 111 |
| 112 | GGGGCAUGGAGCUUCACUCCU | 323 | AGGAGUGAAGCUCCAUGCCCCUU | 112 |
| 113 | GGGCAUGGAGCUUCACUCCUU | 324 | AAGGAGUGAAGCUCCAUGCCCCU | 113 |
| 114 | GGCAUGGAGCUUCACUCCUUA | 325 | UAAGGAGUGAAGCUCCAUGCCCC | 114 |
| 115 | GAGCUUCACUCCUUGCUGGCU | 326 | AGCCAGCAAGGAGUGAAGCUCCA | 115 |
| 116 | AGCUUCACUCCUUGCUGGCCA | 327 | UGGCCAGCAAGGAGUGAAGCUCC | 116 |
| 117 | CUCCUUGCUGGCCAGGGAGUU | 328 | AACUCCCUGGCCAGCAAGGAGUG | 117 |
| 118 | UCCUUGCUGGCCAGGGAGUUA | 329 | UAACUCCCUGGCCAGCAAGGAGU | 118 |
| 119 | GCCAGGGAGUUGGGGACUCAA | 330 | UUGAGUCCCCAACUCCCUGGCCA | 119 |
| 120 | AGGGAGUUGGGGACUCAGAGA | 331 | UCUCUGAGUCCCCAACUCCCUGG | 120 |
| 121 | GGAGUUGGGGACUCAGAGGGA | 332 | UCCCUCUGAGUCCCCAACUCCCU | 121 |
| 122 | GAGUUGGGGACUCAGAGGGAU | 333 | AUCCCUCUGAGUCCCCAACUCCC | 122 |
| 123 | UUGGGGACUCAGAGGGACCAU | 334 | AUGGUCCCUCUGAGUCCCCAACU | 123 |
| 124 | CUCAGAGGGACCACUUGGGGU | 335 | ACCCCAAGUGGUCCCUCUGAGUC | 124 |
| 125 | AAUGGCGGACUCAGUCACAUU | 336 | AAUGUGACUGAGUCCGCCAUUGA | 125 |
| 126 | AUGGCGGACUCAGUCACAUUA | 337 | UAAUGUGACUGAGUCCGCCAUUG | 126 |
| 127 | UGGCGGACUCAGUCACAUUGA | 338 | UCAAUGUGACUGAGUCCGCCAUU | 127 |
| 128 | GGCGGACUCAGUCACAUUGAU | 339 | AUCAAUGUGACUGAGUCCGCCAU | 128 |
| 129 | GACUCAGUCACAUUGACUGAU | 340 | AUCAGUCAAUGUGACUGAGUCCG | 129 |
| 130 | ACUCAGUCACAUUGACUGACA | 341 | UGUCAGUCAAUGUGACUGAGUCC | 130 |
| 131 | GUCACAUUGACUGACGGGGAU | 342 | AUCCCCGUCAGUCAAUGUGACUG | 131 |
| 132 | CACAUUGACUGACGGGGACCA | 343 | UGGUCCCCGUCAGUCAAUGUGAC | 132 |
| 133 | CUGGUGCUGUUGUGUGUAGGU | 344 | ACCUACACACAACAGCACCAGCA | 133 |
| 134 | UGGUGCUGUUGUGUGUAGGUU | 345 | AACCUACACACAACAGCACCAGC | 134 |
| 135 | GCUGUUGUGUGUAGGUCCCCU | 346 | AGGGGACCUACACACAACAGCAC | 135 |
| 136 | CUGUUGUGUGUAGGUCCCCUA | 347 | UAGGGGACCUACACACAACAGCA | 136 |
| 137 | GUUGUGUGUAGGUCCCCUGGA | 348 | UCCAGGGGACCUACACACAACAG | 137 |
| 138 | UGUGUGUAGGUCCCCUGGGGA | 349 | UCCCCAGGGGACCUACACACAAC | 138 |
| 139 | GUGUGUAGGUCCCCUGGGGAU | 350 | AUCCCCAGGGGACCUACACACAA | 139 |
| 140 | ACAAGCAGGCGCCAAUGGUAU | 351 | AUACCAUUGGCGCCUGCUUGUGU | 140 |
| 141 | AAGCAGGCGCCAAUGGUAUCU | 352 | AGAUACCAUUGGCGCCUGCUUGU | 141 |
| 142 | GGCGCCAAUGGUAUCUGGGCA | 353 | UGCCCAGAUACCAUUGGCGCCUG | 142 |
| 143 | AGUUCUUGGAAUAAAAGCAAU | 354 | AUUGCUUUUAUUCCAAGAACUCU | 143 |
| 144 | GUUCUUGGAAUAAAAGCAACU | 355 | AGUUGCUUUUAUUCCAAGAACUC | 144 |
| 145 | UUCUUGGAAUAAAAGCAACCU | 356 | AGGUUGCUUUUAUUCCAAGAACU | 145 |
| 146 | UCUUGGAAUAAAAGCAACCUU | 357 | AAGGUUGCUUUUAUUCCAAGAAC | 146 |
| 147 | GGAAUAAAAGCAACCUCAGAA | 358 | UUCUGAGGUUGCUUUUAUUCCAA | 147 |
| 148 | GAAUAAAAGCAACCUCAGAAU | 359 | AUUCUGAGGUUGCUUUUAUUCCA | 148 |
| 149 | AAUAAAAGCAACCUCAGAACA | 360 | UGUUCUGAGGUUGCUUUUAUUCC | 149 |
| 150 | GUCCCAGGCCCACGAAAGACA | 361 | UGUCUUUCGUGGGCCUGGGACCA | 150 |
| 151 | AGGCCCACGAAAGACGGUGAU | 362 | AUCACCGUCUUUCGUGGGCCUGG | 151 |
| 152 | GGCCCACGAAAGACGGUGACU | 363 | AGUCACCGUCUUUCGUGGGCCUG | 152 |
| 153 | GCCCACGAAAGACGGUGACUU | 364 | AAGUCACCGUCUUUCGUGGGCCU | 153 |
| 154 | GAAAGACGGUGACUCUUGGCU | 365 | AGCCAAGAGUCACCGUCUUUCGU | 154 |
| 155 | AAAGACGGUGACUCUUGGCUU | 366 | AAGCCAAGAGUCACCGUCUUUCG | 155 |
| 156 | CAAGGAGGGGCCAUCUGGAAA | 367 | UUUCCAGAUGGCCCCUCCUUGGA | 156 |
| 157 | GGCCAUCUGGAAACUUGUGGA | 368 | UCCACAAGUUUCCAGAUGGCCCC | 157 |
| 158 | CCAUCUGGAAACUUGUGGACA | 369 | UGUCCACAAGUUUCCAGAUGGCC | 158 |
| 159 | GGAAACUUGUGGACAGAGAAA | 370 | UUUCUCUGUCCACAAGUUUCCAG | 159 |
| 160 | AAACUUGUGGACAGAGAAGAA | 371 | UUCUUCUCUGUCCACAAGUUUCC | 160 |
| 161 | AACUUGUGGACAGAGAAGAAA | 372 | UUUCUUCUCUGUCCACAAGUUUC | 161 |
| 162 | GUGGACAGAGAAGAAGACCAU | 373 | AUGGUCUUCUUCUCUGUCCACAA | 162 |
| 163 | GAAGACCACGACUGGAGAAGU | 374 | ACUUCUCCAGUCGUGGUCUUCUU | 163 |
| 164 | GCUGCAGGAUAUGCUCAGACU | 375 | AGUCUGAGCAUAUCCUGCAGCCU | 164 |
| 165 | UCAGACUCUAGAGGCGUGGAU | 376 | AUCCACGCCUCUAGAGUCUGAGC | 165 |
| 166 | CAGACUCUAGAGGCGUGGACU | 377 | AGUCCACGCCUCUAGAGUCUGAG | 166 |
| 167 | AGACUCUAGAGGCGUGGACCA | 378 | UGGUCCACGCCUCUAGAGUCUGA | 167 |
| 168 | GACUCUAGAGGCGUGGACCAA | 379 | UUGGUCCACGCCUCUAGAGUCUG | 168 |
| 169 | ACUCUAGAGGCGUGGACCAAA | 380 | UUUGGUCCACGCCUCUAGAGUCU | 169 |
| 170 | ACUCCUUGCUGGCCAGGGAGU | 381 | ACUCCCUGGCCAGCAAGGAGUGA | 170 |
| 171 | GGGAGUUGGGGACUCAGAGGA | 382 | UCCUCUGAGUCCCCAACUCCCUG | 171 |
| 172 | UCAGAGGGACCACUUGGGGCU | 383 | AGCCCCAAGUGGUCCCUCUGAGU | 172 |
| 173 | CACUUGGGGCCAGCCAGACUA | 384 | UAGUCUGGCUGGCCCCAAGUGGU | 173 |
| 174 | UGGCCUCAAUGGCGGACUCAA | 385 | UUGAGUCCGCCAUUGAGGCCAGU | 174 |
| 175 | GGCCUCAAUGGCGGACUCAGU | 386 | ACUGAGUCCGCCAUUGAGGCCAG | 175 |
| 176 | GCCUCAAUGGCGGACUCAGUU | 387 | AACUGAGUCCGCCAUUGAGGCCA | 176 |
| 177 | UCAGUCACAUUGACUGACGGA | 388 | UCCGUCAGUCAAUGUGACUGAGU | 177 |
| 178 | UGACUGACGGGGACCAGGGCU | 389 | AGCCCUGGUCCCCGUCAGUCAAU | 178 |
| 179 | GUGCUGGUGCUGUUGUGUGUA | 390 | UACACACAACAGCACCAGCACCA | 179 |
| 180 | AGCAGGCGCCAAUGGUAUCUA | 391 | UAGAUACCAUUGGCGCCUGCUUG | 180 |
| 181 | UGGUAUCUGGGCGGAGCUCAU | 392 | AUGAGCUCCGCCCAGAUACCAUU | 181 |
| 182 | AAGGAGGGGCCAUCUGGAAAU | 393 | AUUUCCAGAUGGCCCCUCCUUGG | 182 |
| 183 | AGGGGCCAUCUGGAAACUUGU | 394 | ACAAGUUUCCAGAUGGCCCCUCC | 183 |
| 184 | CAUCUGGAAACUUGUGGACAA | 395 | UUGUCCACAAGUUUCCAGAUGGC | 184 |
| 185 | UGGAAACUUGUGGACAGAGAA | 396 | UUCUCUGUCCACAAGUUUCCAGA | 185 |
| 186 | UGGACAGAGAAGAAGACCACA | 397 | UGUGGUCUUCUUCUCUGUCCACA | 186 |
| 187 | GAGAAGAAGACCACGACUGGA | 398 | UCCAGUCGUGGUCUUCUUCUCUG | 187 |
| 188 | AGAAGAAGACCACGACUGGAA | 399 | UUCCAGUCGUGGUCUUCUUCUCU | 188 |
| 189 | ACCACGACUGGAGAAGCCCCU | 400 | AGGGGCUUCUCCAGUCGUGGUCU | 189 |
| 190 | AGGCUGCAGGAUAUGCUCAGA | 401 | UCUGAGCAUAUCCUGCAGCCUCG | 190 |
| 191 | GCUUCACUCCUUGCUGGCCAA | 402 | UUGGCCAGCAAGGAGUGAAGCUC | 191 |
| 192 | CACUCCUUGCUGGCCAGGGAA | 403 | UUCCCUGGCCAGCAAGGAGUGAA | 192 |
| 193 | GCUGGCCAGGGAGUUGGGGAU | 404 | AUCCCCAACUCCCUGGCCAGCAA | 193 |
| 194 | CAGGGAGUUGGGGACUCAGAA | 405 | UUCUGAGUCCCCAACUCCCUGGC | 194 |
| 195 | GUUGGGGACUCAGAGGGACCA | 406 | UGGUCCCUCUGAGUCCCCAACUC | 195 |
| 196 | UGGGGACUCAGAGGGACCACU | 407 | AGUGGUCCCUCUGAGUCCCCAAC | 196 |
| 197 | GGGGACUCAGAGGGACCACUU | 408 | AAGUGGUCCCUCUGAGUCCCCAA | 197 |
| 198 | GGGACUCAGAGGGACCACUUA | 409 | UAAGUGGUCCCUCUGAGUCCCCA | 198 |
| 199 | GCCAGACUGGCCUCAAUGGCA | 410 | UGCCAUUGAGGCCAGUCUGGCUG | 199 |
| 200 | CCAGACUGGCCUCAAUGGCGA | 411 | UCGCCAUUGAGGCCAGUCUGGCU | 200 |
| 201 | AUUGACUGACGGGGACCAGGA | 412 | UCCUGGUCCCCGUCAGUCAAUGU | 201 |
| 202 | ACUGACGGGGACCAGGGCUUA | 413 | UAAGCCCUGGUCCCCGUCAGUCA | 202 |
| 203 | GGUCCCCUGGGGACACAAGCA | 414 | UGCUUGUGUCCCCAGGGGACCUA | 203 |
| 204 | GUCCCCUGGGGACACAAGCAA | 415 | UUGCUUGUGUCCCCAGGGGACCU | 204 |
| 205 | GGUAUCUGGGCGGAGCUCACA | 416 | UGUGAGCUCCGCCCAGAUACCAU | 205 |
| 206 | GUAUCUGGGCGGAGCUCACAA | 417 | UUGUGAGCUCCGCCCAGAUACCA | 206 |
| 207 | UAUCUGGGCGGAGCUCACAGA | 418 | UCUGUGAGCUCCGCCCAGAUACC | 207 |
| 208 | CGGAGCUCACAGAGUUCUUGA | 419 | UCAAGAACUCUGUGAGCUCCGCC | 208 |
| 209 | UUCUUGGAAUAAAAGCAAU | 420 | AUUGCUUUUAUUCCAAGAACU | 209 |
| 210 | AGUUCUUGGAAUAAAAGCAAU | 354 | AUUGCUUUUAUUCCAAGAACUUU | 210 |
| 211 | GUUCUUGGAAUAAAAGCAAU | 421 | AUUGCUUUUAUUCCAAGAACUC | 211 |
| 212 | AGUUCUUGGAAUAAAAGCAAU | 354 | AUUGCUUUUAUUCCAAGAACU | 209 |
| 213 | GUUCUUGGAAUAAAAGCAAU | 421 | AUUGCUUUUAUUCCAAGAACUC | 211 |
| 214 | UUCUUGGAAUAAAAGCAAU | 420 | AUUGCUUUUAUUCCAAGAACU | 209 |
| 215 | GUUCUUGGAAUAAAAGCAAU | 421 | AUUGCUUUUAUUCCAAGAAC | 663 |
| 216 | UUCUUGGAAUAAAAGCAAU | 420 | AUUGCUUUUAUUCCAAGAA | 664 |
| 217 | ACAGAGUUCUUGGAAUAAAAG | 666 | AUUGCUUUUAUUCCAAGAACUCU | 665 |
| | CAAU | | CUGU | |

Specifically, uppercase C, G, U, and A represent the base composition of a nucleotide.

A portion of the siRNA sequence was chemically modified and conjugated with a linker-targeting ligand moiety. The resulting siRNA conjugates are as follows:

TABLE 2
Sequences of modified siRNA conjugates

| | Strand | | SEQ ID |
|---|---|---|---|
| No. | type | Sequence (5′→3′) | NO |
| Conjugate 1 | SS | mA*mG*mUmUmCmUfUmGfGfAfAmUmAmAmAmAmGmCmAmA | 422 |
| | | mUGalNAc(L96) | |
| | AS | mA*fU*mUmGmCfUmUmUmUmAmUmUmCfCmAfAmGmAmAmC | 423 |
| | | mU*mC*mU | |
| Conjugate 2 | SS | mG*mU*mUmCmUmUfGmGfAfAfUmAmAmAmAmGmCmAmAmC | 424 |
| | | mUGalNAc(L96) | |
| | AS | mA*fG*mUmUmGfCmUmUmUmUmAmUmUfCmCfAmAmGmAmA | 425 |
| | | mC*mU*mC | |
| Conjugate 3 | SS | mU*mC*mUmUmGmGfAmAfUfAfAmAmAmGmCmAmAmCmCmU | 426 |
| | | mUGalNAc(L96) | |
| | AS | mA*fA*mGmGmUfUmGmCmUmUmUmUmAfUmUfCmCmAmAmG | 427 |
| | | mA*mA*mC | |
| Conjugate 4 | SS | mG*mG*mAmAmUmAfAmAfAfGfCmAmAmCmCmUmCmAmGmA | 428 |
| | | mAGalNAc(L96) | |
| | AS | mU*fU*mCmUmGfAmGmGmUmUmGmCmUfUmUfUmAmUmUmC | 429 |
| | | mC*mA*mA | |
| Conjugate 5 | SS | mA*mG*mUmUmCmUfUmGfGfAfAmUmAmAmAmAmGmCmAmA | 422 |
| | | mUGalNAc(L96) | |
| | AS | mA*fU*mUmGmCfUmUfUfUmAmUmUmCfCmAfAmGmAmAmCm | 430 |
| | | U*mC*mU | |
| Conjugate 6 | SS | mG*mU*mUmCmUmUfGmGfAfAfUmAmAmAmAmGmCmAmAmC | 424 |
| | | mUGalNAc(L96) | |
| | AS | mA*fG*mUmUmGfCmUfUfUmUmAmUmUfCmCfAmAmGmAmAm | 431 |
| | | C*mU*mC | |
| Conjugate 7 | SS | mU*mC*mUmUmGmGfAmAfUfAfAmAmAmGmCmAmAmCmCmU | 426 |
| | | mUGalNAc(L96) | |
| | AS | mA*fA*mGmGmUfUmGfCfUmUmUmUmAfUmUfCmCmAmAmGm | 432 |
| | | A*mA*mC | |
| Conjugate 8 | SS | mG*mG*mAmAmUmAfAmAfAfGfCmAmAmCmCmUmCmAmGmA | 428 |
| | | mAGalNAc(L96) | |
| | AS | mU*fU*mCmUmGfAmGfGfUmUmGmCmUfUmUfUmAmUmUmCm | 433 |
| | | C*mA*mA | |
| Conjugate 9 | SS | mG*mA*mAmUmAmAfAmAfGfCfAmAmCmCmUmCmAmGmAmA | 434 |
| | | mUGalNAc(L96) | |
| | AS | mA*fU*mUmCmUfGmAfGfGmUmUmGmCfUmUfUmUmAmUmUm | 435 |
| | | C*mC*mA | |
| Conjugate | SS | mA*mA*mUmAmAmAfAmGfCfAfAmCmCmUmCmAmGmAmAmC | 436 |
| 10 | | mAGalNAc(L96) | |
| | AS | mU*fG*mUmUmCfUmGfAfGmGmUmUmGfCmUfUmUmUmAmUm | 437 |
| | | U*mC*mC | |
| Conjugate | SS | mG*mA*mAmUmAmAfAmAfGfCfAmAmCmCmUmCmAmGmAmA | 434 |
| 11 | | mUGalNAc(L96) | |
| | AS | mA*fU*mUmCmUfGmAmGmGmUmUmGmCfUmUfUmUmAmUmU | 438 |
| | | mC*mC*mA | |
| Conjugate | SS | mA*mA*mUmAmAmAfAmGfCfAfAmCmCmUmCmAmGmAmAmC | 436 |
| 12 | | mAGalNAc(L96) | |
| | AS | mU*fG*mUmUmCfUmGmAmGmGmUmUmGfCmUfUmUmUmAmU | 439 |
| | | mU*mC*mC | |
| Conjugate | SS | mA*mG*mUmUmCmUfUmGfGfAfAmUmAmAmAmAmGmCmAmA | 440 |
| 13 | | mUGalNAc(L96) | |
| | AS | mA*fU*mUmGmCfUmUmUmUfAmUmUmCfCmAfAmGmAmAmCm | 441 |
| | | U*mC*mU | |
| Conjugate | SS | fA*mG*fUmUfCmUfUmGfGfAfAmUfAmAfAmAfGmCfAmAfUGalN | 442 |
| 14 | | Ac(L96) | |
| | AS | mA*fU*mUfGmCfUmUfUmUfAmUmUmCfCmAfAmGfAmAfCmU* | 443 |
| | | mC*mU | |
| Conjugate | SS | mA*mG*mUmUmCmUfUmGfGfAfAmUmAmAmAmAmGmCmAmA | 440 |
| 15 | | mUGalNAc(L96) | |
| | AS | mA*fU*mUmGmCfUmUmUfUmAmUmUmCfCmAfAmGmAmAmCm | 444 |
| | | U*mC*mU | |
| Conjugate | SS | mA*mG*mUmUmCmUmUmGfGfAfAmUmAmAmAmAmGmCmAm | 445 |
| 16 | | AmUGalNAc(L96) | |
| | AS | mA*dT*mUmGdCmUdTmUmUmAmUtnTmCfCmAmAmGmAmAmC | 446 |
| | | mU*mC*mU | |
| Conjugate | SS | mA*mG*mUmUmCmUfUmGfGfAfAmUmAmAmAmAmGmCmAmA | 440 |
| 17 | | mUGalNAc(L96) | |
| | AS | mA*fU*mUmGfCfUmUmUmUmAmUmUmCfCmAfAmGmAmAmCm | 447 |
| | | U*mC*mU | |
| Conjugate | SS | mA*mG*mUmUmCmUfUmGfGfAfAmUmAmAmAmAmGmCmAmA | 440 |
| 18 | | mUGalNAc(L96) | |
| | AS | mA*fU*mUmGfCfUmUfUfUmAmUmUmCfCmAfAmGmAmAmCmU | 448 |
| | | *mC*mU | |
| Conjugate | SS | mA*mG*mUmUmCmUfUmGfGfAfAmUmAmAmAmAmGmCmAmA | 440 |
| 19 | | mUGalNAc(L96) | |
| | AS | eVPmA*fU*mUmGmCfUmUmUmUfAmUmUmCfCmAfAmGmAmA | 449 |
| | | mCmU*mC*mU | |
| Conjugate | SS | fA*mG*fUmUfCmUfUmGfGfAfAmUfAmAfAmAfGmCfAmAfUGalN | 442 |
| 20 | | Ac(L96) | |
| | AS | eVPmA*fU*mUfGmCfUmUfUmUfAmUmUmCfCmAfAmGfAmAfCm | 450 |
| | | U*mC*mU | |
| Conjugate | SS | mA*mG*mUmUmCmUfUmGfGfAfAmUmAmAmAmAmGmCmAmA | 440 |
| 21 | | mUGalNAc(L96) | |
| | AS | eVPmA*fU*mUmGmCfUmUmUfUmAmUmUmCfCmAfAmGmAmA | 451 |
| | | mCmU*mC*mU | |
| Conjugate | SS | mA*mG*mUmUmCmUmUmGfGfAfAmUmAmAmAmAmGmCmAm | 445 |
| 22 | | AmUGalNAc(L96) | |
| | AS | eVPmA*dT*mUmGdCmUdTmUmUmAmUtnTmCfCmAmAmGmAm | 452 |
| | | AmCmU*mC*mU | |
| Conjugate | SS | mA*mG*mUmUmCmUfUmGfGfAfAmUmAmAmAmAmGmCmAmA | 440 |
| 23 | | mUGalNAc(L96) | |
| | AS | eVPmA*fU*mUmGfCfUmUmUmUmAmUmUmCfCmAfAmGmAmA | 453 |
| | | mCmU*mC*mU | |
| Conjugate | SS | mA*mG*mUmUmCmUfUmGfGfAfAmUmAmAmAmAmGmCmAmA | 440 |
| 24 | | mUGalNAc(L96) | |
| | AS | eVPmA*fU*mUmGfCfUmUfUfUmAmUmUmCfCmAfAmGmAmAmC | 454 |
| | | mU*mC*mU | |
| Conjugate | SS | mG*mU*mUmCmUmUmGfGfAfAmUmAmAmAmAmGmCmAmAm | 455 |
| 25 | | U*GalNAc(L96) | |
| | AS | mA*fU*mUmGmCfUmUmUmUmAmUmUmCfCmAfAmGmAmAmC | 456 |
| | | *mU*mC | |
| Conjugate | SS | mG*mU*mUmCmUmUmGfGfAfAmUmAmAmAmAmGmCmAmAm | 455 |
| 26 | | U*GalNAc(L96) | |
| | AS | mA*fU*mUmGmCfUmUmUmUmAmUmUmCfCmAfAmGmAmA*m | 457 |
| | | C*mU | |
| Conjugate | SS | mU*mU*mCmUmUmGfGfAfAmUmAmAmAmAmGmCmAmAmU*G | 458 |
| 27 | | alNAc(L96) | |
| | AS | eVPmA*fU*mUmGmCfUmUmUmUmAmUmUmCfCmAfAmGmAmA | 456 |
| | | mC*mU*mC | |
| Conjugate | SS | mU*mU*mCmUmUmGfGfAfAmUmAmAmAmAmGmCmAmAmU*G | 458 |
| 28 | | alNAc(L96) | |
| | AS | eVPmA*fU*mUmGmCfUmUmUmUmAmUmUmCfCmAfAmGmAmA | 460 |
| | | *mC*mU | |
| Conjugate | SS | mA*mG*mUmUmCmUfUmGfGfAfAmUmAmAmAmAmGmCmAmA | 422 |
| 29 | | mUGalNAc(L96) | |
| | AS | mA*fU*mUfGmCfUmUfUfUmAmUmUmCfCmAfAmGmAmAmCmU | 461 |
| | | *mC*mU | |
| Conjugate | SS | mA*mG*mUmUmCmUfUmGfGfAfAmUmAmAmAmAmGmCmAmA | 422 |
| 30 | | mUGalNAc(L96) | |
| | AS | mA*fU*mUmGmCmUmUmUmUmAmUmUmCfCmAfAmGmAmAm | 462 |
| | | CmU*mC*mU | |
| Conjugate | SS | mA*mG*mUmUmCmUfUmGfGfAfAmUmAmAmAmAmGmCmAmA | 422 |
| 31 | | mUGalNAc(L96) | |
| | AS | mA*fU*mUfGmCfUmUmUmUmAmUmUmCfCmAfAmGmAmAmCm | 463 |
| | | U*mC*mU | |
| Conjugate | SS | mA*mG*mUmUmCmUmUmGfGfAfAmUmAmAmAmAmGmCmAm | 464 |
| 32 | | AmUGalNAc(L96) | |
| | AS | mA*fU*mUmGmCfUmUmUmUmAmUmUmCfCmAfAmGmAmAmC | 423 |
| | | mU*mC*mU | |
| Conjugate | SS | mA*mG*mUmUmCmUmUmGfGfAfAmUmAmAmAmAmGmCmAm | 464 |
| 33 | | AmUGalNAc(L96) | |
| | AS | mA*fU*mUmGmCmUmUmUmUmAmUmUmCfCmAfAmGmAmAm | 462 |
| | | CmU*mC*mU | |
| Conjugate | SS | mA*mG*mUmUmCmUmUmGfGfAfAmUmAmAmAmAmGmCmAm | 464 |
| 34 | | AmUGalNAc(L96) | |
| | AS | mA*fU*mUfGmCfUmUmUmUmAmUmUmCfCmAfAmGmAmAmCm | 463 |
| | | U*mC*mU | |
| Conjugate | SS | mG*mU*mUmCmUmUmGfGfAfAmUmAmAmAmAmGmCmAmAm | 465 |
| 35 | | UGalNAc(L96) | |
| | AS | mA*fU*mUmGmCmUmUmUmUmAmUmUmCfCmAfAmGmAmAm | 466 |
| | | C*mU*mC | |
| Conjugate | SS | mG*mU*mUmCmUmUmGfGfAfAmUmAmAmAmAmGmCmAmAm | 465 |
| 36 | | UGalNAc(L96) | |
| | AS | mA*fU*mUfGmCfUmUmUmUmAmUmUmCfCmAfAmGmAmAmC* | 467 |
| | | mU*mC | |
| Conjugate | SS | mG*mA*mAmUmAmAfAmAfGfCfAmAmCmCmUmCmAmGmAmA | 468 |
| 37 | | mAGalNAc(L96) | |
| | AS | mU*fU*mUmCmUfGmAfGfGmUmUmGmCfUmUfUmUmAmUmUm | 469 |
| | | C*mC*mA | |
| Conjugate | SS | mG*mA*mAmCmAmAfAmAfGfCfAmAmCmCmUmCmAmGmAmA | 470 |
| 38 | | mAGalNAc(L96) | |
| | AS | mU*fU*mUmCmUfGmAfGfGmUmUmGmCfUmUfUmUmGmUmUm | 471 |
| | | C*mU*mU | |
| Conjugate | SS | mG*mA*mAmCmAfAmAfAfGfCmAmAmCmCmUmCmAmGmAmA | 472 |
| 39 | | GalNAc(L96) | |
| | AS | mU*fU*mCmUmGfAmGfGfUmUmGmCmUfUmUfUmGmUmUmC* | 473 |
| | | mU*mU | |
| Conjugate | SS | mG*mA*mAmCmAmAfAmAfGfCfAmAmCmCmUmCmAmGmAmA | 470 |
| 40 | | mAGalNAc(L96) | |
| | AS | mU*fU*mUmCmUfGmAmGmGmUmUmGmCfUmUfUmUmGmUmU | 474 |
| | | mC*mU*mU | |
| Conjugate | SS | mG*mA*mAmCmAfAmAfAfGfCmAmAmCmCmUmCmAmGmAmA | 472 |
| 41 | | GalNAc(L96) | |
| | AS | mU*fU*mCmUmGfAmGmGmUmUmGmCmUfUmUfUmGmUmUmC | 475 |
| | | *mU*mU | |
| Conjugate | SS | mG*mA*mAmUmAmAmAmAfGfCfAmAmCmCmUmCmAmGmAm | 476 |
| 42 | | AmAGalNAc(L96) | |
| | AS | mU*fU*mUmCmUfGmAmGmGmUmUmGmCfUmUfUmUmAmUmU | 477 |
| | | mC*mC*mA | |
| Conjugate | SS | mG*mA*mAmCmAmAmAmAfGfCfAmAmCmCmUmCmAmGmAmA | 478 |
| 43 | | mAGalNAc(L96) | |
| | AS | mU*fU*mUmCmUfGmAmGmGmUmUmGmCfUmUfUmUmGmUmU | 474 |
| | | mC*mU*mU | |
| Conjugate | SS | mG*mA*mAmCmGmAmAmAfGfUfAmAmCmCmUmCmAmGmAm | 479 |
| 44 | | AmAGalNAc(L96) | |
| | AS | mU*fU*mUmCmUfGmAmGmGmUmUmGmCfUmUfUmCmGmUmU | 480 |
| | | mC*mU*mU | |
| Conjugate | SS | mG*mA*mAmCmAmGmAmAfGfUfAmAmCmCmUmCmAmGmAm | 481 |
| 45 | | AmAGalNAc(L96) | |
| | AS | mU*fU*mUmCmUfGmAmGmGmUmUmGmCfUmUfCmUmGmUmU | 482 |
| | | mC*mU*mU | |
| Conjugate | SS | mG*mA*mAmUmAmAmAfAfGfCmAmAmCmCmUmCmAmGmAm | 483 |
| 46 | | AGalNAc(L96) | |
| | AS | mU*fU*mCmUmGfAmGmGmUmUmGmCmUfUmUfUmAmUmUmC | 484 |
| | | *mU*mU | |
| Conjugate | SS | mC*mA*mAmAfAmGfCfAfAmCmCmUmCmAmGmAmAmCmAGal | 485 |
| 47 | | NAc(L96) | |
| | AS | mU*fG*mUmUmCfUmGfAfGmGmUmUmGfCmUfUmUmUmG*mU* | 486 |
| | | mU | |
| Conjugate | SS | mC*mA*mAmAfAmGfCfAfAmCmCmUmCmAmGmAmAmCmAGal | 485 |
| 48 | | NAc(L96) | |
| | AS | mU*fG*mUmUmCfUmGmAmGmGmUmUmGfCmUfUmUmUmG*m | 487 |
| | | U*mU | |
| Conjugate | SS | mC*mA*mAmGfAmGfCfAfAmCmCmUmCmAmGmAmAmCmAGal | 488 |
| 49 | | NAc(L96) | |
| | AS | mU*fG*mUmUmCfUmGmAmGmGmUmUmGfCmUfCmUmUmG*m | 489 |
| | | U*mU | |
| Conjugate | SS | mC*mA*mAmAmAmGfCfAfAmCmCmUmCmAmGmAmAmCmAGal | 490 |
| 50 | | NAc(L96) | |
| | AS | mU*fG*mUmUmCfUmGmAmGmGmUmUmGfCmUfUmUmUmG*m | 487 |
| | | U*mU | |
| Conjugate | SS | mC*mA*mAmAmAmGfCfAfAmUmCmUmCmAmGmAmAmCmAGal | 491 |
| 51 | | NAc(L96) | |
| | AS | mU*fG*mUmUmCfUmGmAmGmGmUmUmGfCmUfUmUmUmG*m | 487 |
| | | U*mU | |
| Conjugate | SS | mC*mA*mAmGmAmGfCfAfAmCmCmUmCmAmGmAmAmCmAGal | 492 |
| 52 | | NAc(L96) | |
| | AS | mU*fG*mUmUmCfUmGmAmGmGmUmUmGfCmUfCmUmUmG*m | 489 |
| | | U*mU | |
| Conjugate | SS | mA*mG*mUmUmCmUfUmGfGfAfAmUmAmAmAmAmGmCmAmA | 493 |
| 53 | | mAGalNAc(L96) | |
| | AS | mU*fU*mUmGmCfUmUmUmUmAmUmUmCfCmAfAmGmAmAmC | 494 |
| | | mU*mC*mU | |
| Conjugate | SS | mA*mG*mUmUmCmUfUmGfGfAfAmUmAmAmAmAmGmCmAmA | 493 |
| 54 | | mAGalNAc(L96) | |
| | AS | mU*fU*mUmGmCfUmUfUfUmAmUmUmCfCmAfAmGmAmAmCm | 495 |
| | | U*mC*mU | |
| Conjugate | SS | mA*mG*mUmUmCmUmUmGfGfAfAmUmAmAmAmAmGmCmAm | 496 |
| 55 | | AmAGalNAc(L96) | |
| | AS | mU*fU*mUmGmCfUmUmUmUmAmUmUmCfCmAfAmGmAmAmC | 494 |
| | | mU*mC*mU | |
| Conjugate | SS | mA*mG*mUmUmCdTfUmGfGfAfAmUmAmAmAmAmGmCmAmA | 497 |
| 56 | | mUGalNAc(L96) | |
| | AS | mA*fU*mUmGmCfUmUmUmUmAmUmUmCfCmAfAmGmAmAmC | 423 |
| | | mU*mC*mU | |
| Conjugate | SS | mA*mG*mUmUmCdTfUmGfGfAfAmUmAmAmAmAmGmCmAmA | 497 |
| 57 | | mUGalNAc(L96) | |
| | AS | mA*fU*mUmGmCfUmUfUfUmAmUmUmCfCmAfAmGmAmAmCm | 430 |
| | | U*mC*mU | |
| Conjugate | SS | mA*mG*mUmUmCdTmUmGfGfAfAmUmAmAmAmAmGmCmAmA | 498 |
| 58 | | mUGalNAc(L96) | |
| | AS | mA*fU*mUmGmCfUmUmUmUmAmUmUmCfCmAfAmGmAmAmC | 423 |
| | | mU*mC*mU | |
| Conjugate | SS | mG*mG*mUmUmCmUfUmGfGfAfAmUmAmAmAmAmGmCmAmA | 499 |
| 59 | | mUGalNAc(L96) | |
| | AS | mA*fU*mUmGmCfUmUmUmUmAmUmUmCfCmAfAmGmAmAmC | 423 |
| | | mU*mC*mU | |
| Conjugate | SS | mG*mG*mUmUmCmUfUmGfGfAfAmUmAmAmAmAmGmCmAmA | 499 |
| 60 | | mUGalNAc(L96) | |
| | AS | mA*fU*mUmGmCfUmUfUfUmAmUmUmCfCmAfAmGmAmAmCm | 430 |
| | | U*mC*mU | |
| Conjugate | SS | mG*mG*mUmUmCmUmUmGfGfAfAmUmAmAmAmAmGmCmAm | 500 |
| 61 | | AmUGalNAc(L96) | |
| | AS | mA*fU*mUmGmCfUmUmUmUmAmUmUmCfCmAfAmGmAmAmC | 423 |
| | | mU*mC*mU | |
| Conjugate | SS | tnA*mG*mUmUmCmUfUmGfGfAfAmUmAmAmAmAmGmCmAmA | 501 |
| 62 | | mUGalNAc(L96) | |
| | AS | mA*fU*mUmGmCfUmUmUmUmAmUmUmCfCmAfAmGmAmAmC | 423 |
| | | mU*mC*mU | |
| Conjugate | SS | tnA*mG*mUmUmCmUfUmGfGfAfAmUmAmAmAmAmGmCmAmA | 501 |
| 63 | | mUGalNAc(L96) | |
| | AS | mA*fU*mUmGmCfUmUfUfUmAmUmUmCfCmAfAmGmAmAmCm | 430 |
| | | U*mC*mU | |
| Conjugate | SS | tnA*mG*mUmUmCmUmUmGfGfAfAmUmAmAmAmAmGmCmAm | 502 |
| 64 | | AmUGalNAc(L96) | |
| | AS | mA*fU*mUmGmCfUmUmUmUmAmUmUmCfCmAfAmGmAmAmC | 423 |
| | | mU*mC*mU | |
| Conjugate | SS | mA*mG*mUmUmCmUfUmGfGfAfAmUmAmAmAmAmGmCmAmA | 422 |
| 65 | | mUGalNAc(L96) | |
| | AS | mA*fU*mUmGmCfUmUmUmUmAmUmUmCfCmAfAmGmAmAmC | 503 |
| | | mU*tnC*mU | |
| Conjugate | SS | mA*mG*mUmUmCmUfUmGfGfAfAmUmAmAmAmAmGmCmAmA | 422 |
| 66 | | mUGalNAc(L96) | |
| | AS | mA*fU*mUmGmCfUmUfUfUmAmUmUmCfCmAfAmGmAmAmCm | 504 |
| | | U*tnC*mU | |
| Conjugate | SS | mA*mG*mUmUmCmUmUmGfGfAfAmUmAmAmAmAmGmCmAm | 464 |
| 67 | | AmUGalNAc(L96) | |
| | AS | mA*fU*mUmGmCfUmUmUmUmAmUmUmCfCmAfAmGmAmAmC | 503 |
| | | mU*tnC*mU | |
| Conjugate | SS | mA*mG*mUmUmCmUfUmGfGfAfAmUmAmAmAmAmGmCmAmA | 422 |
| 68 | | mUGalNAc(L96) | |
| | AS | mA*fU*mUmGmCfUmUmUmUmAmUmUmCfCmAfAmGmAmAmC | 505 |
| | | mU*ml*mU | |
| Conjugate | SS | mA*mG*mUmUmCmUfUmGfGfAfAmUmAmAmAmAmGmCmAmA | 422 |
| 69 | | mUGalNAc(L96) | |
| | AS | mA*fU*mUmGmCfUmUfUfUmAmUmUmCfCmAfAmGmAmAmCm | 506 |
| | | U*ml*mU | |
| Conjugate | SS | mA*mG*mUmUmCmUmUmGfGfAfAmUmAmAmAmAmGmCmAm | 464 |
| 70 | | AmUGalNAc(L96) | |
| | AS | mA*fU*mUmGmCfUmUmUmUmAmUmUmCfCmAfAmGmAmAmC | 505 |
| | | mU*ml*mU | |
| Conjugate | SS | mG*mU*mUmCmUfUmGfGfAfAmUmAmAmAmAmGmCmAmAmA | 507 |
| 71 | | *GalNAc(L96) | |
| | AS | mU*fU*mUmGmCfUmUmUmUmAmUmUmCfCmAfAmGmAmAmC | 508 |
| | | *mU*mC | |
| Conjugate | SS | mG*mU*mUmCmUfUmGfGfAfAmUmAmAmAmAmGmCmAmAmA | 507 |
| 72 | | *GalNAc(L96) | |
| | AS | mU*fU*mUmGmCfUmUfUfUmAmUmUmCfCmAfAmGmAmAmC*m | 509 |
| | | U*mC | |
| Conjugate | SS | mG*mU*mUmCmUmUmGfGfAfAmUmAmAmAmAmGmCmAmAm | 510 |
| 73 | | A*GalNAc(L96) | |
| | AS | mU*fU*mUmGmCfUmUmUmUmAmUmUmCfCmAfAmGmAmAmC | 508 |
| | | *mU*mC | |
| Conjugate | SS | mG*mU*mUmCdTfUmGfGfAfAmUmAmAmAmAmGmCmAmAmU* | 511 |
| 74 | | GalNAc(L96) | |
| | AS | mA*fU*mUmGmCfUmUmUmUmAmUmUmCfCmAfAmGmAmAmC | 456 |
| | | *mU*mC | |
| Conjugate | SS | mG*mU*mUmCdTfUmGfGfAfAmUmAmAmAmAmGmCmAmAmU* | 511 |
| 75 | | GalNAc(L96) | |
| | AS | mA*fU*mUmGmCfUmUfUfUmAmUmUmCfCmAfAmGmAmAmC*m | 512 |
| | | U*mC | |
| Conjugate | SS | mG*mU*mUmCdTmUmGfGfAfAmUmAmAmAmAmGmCmAmAmU | 513 |
| 76 | | *GalNAc(L96) | |
| | AS | mA*fU*mUmGmCfUmUmUmUmAmUmUmCfCmAfAmGmAmAmC | 456 |
| | | *mU*mC | |
| Conjugate | SS | tnG*mU*mUmCmUfUmGfGfAfAmUmAmAmAmAmGmCmAmAmU | 514 |
| 77 | | *GalNAc(L96) | |
| | AS | mA*fU*mUmGmCfUmUmUmUmAmUmUmCfCmAfAmGmAmAmC | 456 |
| | | *mU*mC | |
| Conjugate | SS | tnG*mU*mUmCmUfUmGfGfAfAmUmAmAmAmAmGmCmAmAmU | 514 |
| 78 | | *GalNAc(L96) | |
| | AS | mA*fU*mUmGmCfUmUfUfUmAmUmUmCfCmAfAmGmAmAmC*m | 456 |
| | | U*mC | |
| Conjugate | SS | tnG*mU*mUmCmUmUmGfGfAfAmUmAmAmAmAmGmCmAmAm | 515 |
| 79 | | U*GalNAc(L96) | |
| | AS | mA*fU*mUmGmCfUmUmUmUmAmUmUmCfCmAfAmGmAmAmC | 456 |
| | | *mU*mC | |
| Conjugate | SS | mG*mU*mUmCmUfUmGfGfAfAmUmAmAmAmAmGmCmAmAmU | 516 |
| 80 | | *GalNAc(L96) | |
| | AS | mA*fU*mUmGmCfUmUmUmUmAmUmUmCfCmAfAmGmAmAmC | 517 |
| | | *tnU*mC | |
| Conjugate | SS | mG*mU*mUmCmUfUmGfGfAfAmUmAmAmAmAmGmCmAmAmU | 516 |
| 81 | | *GalNAc(L96) | |
| | AS | mA*fU*mUmGmCfUmUfUfUmAmUmUmCfCmAfAmGmAmAmC*tn | 518 |
| | | U*mC | |
| Conjugate | SS | mG*mU*mUmCmUmUmGfGfAfAmUmAmAmAmAmGmCmAmAm | 519 |
| 82 | | U*GalNAc(L96) | |
| | AS | mA*fU*mUmGmCfUmUmUmUmAmUmUmCfCmAfAmGmAmAmC | 517 |
| | | *tnU*mC | |
| Conjugate | SS | mG*mU*mUmCmUfUmGfGfAfAmUmAmAmAmAmGmCmAmAmU | 516 |
| 83 | | *GalNAc(L96) | |
| | AS | mA*fU*mUmGmCfUmUmUmUmAmUmUmCfCmAfAmGmAmAmC | 520 |
| | | *mI*mC | |
| Conjugate | SS | mG*mU*mUmCmUfUmGfGfAfAmUmAmAmAmAmGmCmAmAmU | 516 |
| 84 | | *GalNAc(L96) | |
| | AS | mA*fU*mUmGmCfUmUfUfUmAmUmUmCfCmAfAmGmAmAmC*m | 521 |
| | | I*mC | |
| Conjugate | SS | mG*mU*mUmCmUmUmGfGfAfAmUmAmAmAmAmGmCmAmAm | 519 |
| 85 | | U*GalNAc(L96) | |
| | AS | mA*fU*mUmGmCfUmUmUmUmAmUmUmCfCmAfAmGmAmAmC | 520 |
| | | *mI*mC | |
| Conjugate | SS | mG*mU*mUmCmUfUmGfGfAfAmUmAmAmAmAmGmCmAmAmU | 516 |
| 86 | | *GalNAc(L96) | |
| | AS | mA*fU*mUmGmCmUmUmUmUmAmUmUmCfCmAfAmGmAmAm | 466 |
| | | C*mU*mC | |
| Conjugate | SS | mG*mU*mUmCmUfUmGfGfAfAmUmAmAmAmAmGmCmAmAmU | 516 |
| 87 | | *GalNAc(L96) | |
| | AS | mA*fU*mUmGmCmUmUfUfUmAmUmUmCfCmAfAmGmAmAmC* | 522 |
| | | mU*mC | |
| Conjugate | SS | mG*mU*mUmCmUmUmGfGfAfAmUmAmAmAmAmGmCmAmAm | 519 |
| 88 | | U*GalNAc(L96) | |
| | AS | mA*fU*mUmGmCmUmUmUmUmAmUmUmCfCmAfAmGmAmAm | 466 |
| | | C*mU*mC | |
| Conjugate | SS | mU*mU*mCmUmUmGfGfAfAmUmAmAmAmAmGmCmAmAmU*G | 523 |
| 89 | | alNAc(L96) | |
| | AS | mA*fU*mUmGmCfUmUmUmUmAmUmUmGfCmAfAmGmAmA*m | 524 |
| | | C*mU | |
| Conjugate | SS | mG*mU*mUmCmUfUmGfGfAfAmUmAmAmAmAmGmCmAmAmU | 516 |
| 90 | | *GalNAc(L96) | |
| | AS | mA*fU*mUmGmCfUmUmUmUmAmUmUmCfCmAfAmGmAmAmC | 456 |
| | | *mU*mC | |
| Conjugate | SS | mG*mU*mUmCmUfUmGfGfAfAmUmAmAmAmAmGmCmAmAmU | 516 |
| 91 | | *GalNAc(L96) | |
| | AS | mA*fU*mUmGmCfUmUfUfUmAmUmUmCfCmAfAmGmAmAmC*m | 512 |
| | | U*mC | |
| Conjugate | SS | mA*mG*mUmUmCmUmUmGfGfAfAmUmAmAmAmAmGmCmAm | 464 |
| 92 | | AmUGalNAc(L96) | |
| | AS | eVPmA*fU*mUmGmCfUmUmUmUmAmUmUmCfCmAfAmGmAmA | 525 |
| | | mCmU*mC*mU | |
| Conjugate | SS | mA*mG*mUmUmCmUfUmGfGfAfAmUmAmAmAmAmGmCmAmA | 422 |
| 93 | | mUGalNAc(L96) | |
| | AS | eVPmA*fU*mUmGmCfUmUmUmUmAmUmUmCfCmAfAmGmAmA | 525 |
| | | mCmU*mC*mU | |
| Conjugate | SS | mA*mG*mUmUmCmUfUmGfGfAfAmUmAmAmAmAmGmCmAmA | 422 |
| 94 | | mUGalNAc(L96) | |
| | AS | eVPmA*fU*mUmGmCfUmUfUfUmAmUmUmCfCmAfAmGmAmAm | 526 |
| | | CmU*mC*mU | |
| Conjugate | SS | mA*mA*mUmAmAmAmAmGfCfAfAmCmCmUmCmAmGmAmAmC | 527 |
| 95 | | mAGalNAc(L96) | |
| | AS | mU*fG*mUmUmCfUmGmGmGmGmUmUmGfCmUfUmUmUmAmU | 528 |
| | | mU*mC*mC | |
| Conjugate | SS | mA*mG*mUmUmCmUfUmGfGfAfAmUmAmAmAmAmGmCmAmA | 422 |
| 96 | | mUGalNAc(L96) | |
| | AS | eVPmA*fU*mUmGmCfUmUfUfUfAmUmUmCfCmAfAmGmAmAmC | 529 |
| | | mU*mC*mU | |
| Conjugate | SS | mA*mG*mUmUmCmUfUmGfGfAfAmUmAmAfAmAmGmCmA*mA | 530 |
| 97 | | *mUGalNAc(L96) | |
| | AS | eVPmA*fU*mUmGmCfUmUfUfUfAmUmUmCfCmAfAmGmAmAmC | 529 |
| | | mU*mC*mU | |
| Conjugate | SS | mA*mG*mUmUmCmUfUmGfGfAfAmUmAmAmAmAmGmCmAmA | 422 |
| 98 | | mUGalNAc(L96) | |
| | AS | mA*fU*mUmGmCfUmUfUfUfAmUmUmCfCmAfAmGmAmAmCmU | 531 |
| | | *mC*mU | |
| Conjugate | SS | mA*mG*mUmUmCmUfUmGfGfAfAmUmAmAfAmAmGmCmA*mA | 530 |
| 99 | | *mUGalNAc(L96) | |
| | AS | mA*fU*mUmGmCfUmUfUfUfAmUmUmCfCmAfAmGmAmAmCmU | 531 |
| | | *mC*mU | |
| Conjugate | SS | mA*mG*mUmUmCmUfUmGfGmAfAmUfAmAfAmAmGmCmAmA | 532 |
| 100 | | mUGalNAc(L96) | |
| | AS | mA*fU*fUfGmCfUmUfUmUfAmUfUmCfCmAfAmGfAmAfCmU*mC | 533 |
| | | *mU | |
| Conjugate | SS | tnA*mG*mUmUmCmUfUmGfGfAfAmUmAmAmAmAmGmCmAmA | 501 |
| 101 | | mUGalNAc(L96) | |
| | AS | mA*fU*mUmGmCfUmUmUmUmAmUmUmCfCmAfAmGmAmAmCt | 534 |
| | | nU*mC*mU | |
| Conjugate | SS | tnA*mG*mUmUmCmUfUmGfGfAfAmUmAmAmAmAmGmCmAmA | 501 |
| 102 | | mUGalNAc(L96) | |
| | AS | mA*fU*mUmGmCfUmUmUmUmAmUmUmCfCmAfAmGmAmAmCt | 535 |
| | | nU*tnC*mU | |
| Conjugate | SS | tnA*mG*mUmUmCmUfUmGfGfAfAmUmAmAmAmAmGmCmAmA | 501 |
| 103 | | mUGalNAc(L96) | |
| | AS | mA*fU*mUmGmCfUmUmUmUmAmUmUmCfCmAfAmGmAmAmCt | 536 |
| | | nU*mC*tnU | |
| Conjugate | SS | tnA*mG*mUmUmCmUfUmGfGfAfAmUmAmAmAmAmGmCmAmA | 501 |
| 104 | | mUGalNAc(L96) | |
| | AS | mA*fU*mUmGmCfUmUmUmUmAmUmUmCfCmAfAmGmAmAmCt | 537 |
| | | nU*tnC*tnU | |
| Conjugate | SS | mC*mA*mAmAfAmGfCfAfAmCmCmUmCmAmGmAmAmCmUGal | 538 |
| 105 | | NAc(L96) | |
| | AS | mA*fG*mUmUmCfUmGfAfGmGmUmUmGfCmUfUmUmUmG*mU* | 539 |
| | | mU | |
| Conjugate | SS | mC*mA*mAmAfAmGfCfAfAmCmCmUmCmAmGmAmAmCmUGal | 538 |
| 106 | | NAc(L96) | |
| | AS | mA*fG*mUmUmCfUmGmAmGmGmUmUmGfCmUfUmUmUmG*m | 540 |
| | | U*mU | |
| Conjugate | SS | mCmAmAmAmAmGfCfAfAmUmCmUmCmAmGmAmAmCmUGalN | 541 |
| 107 | | Ac(L96) | |
| | AS | mA*fG*mUmUmCfUmGmAmGmGmUmUmGfCmUfUmUmUmG*m | 540 |
| | | U*mU | |
| Conjugate | SS | mC*mA*mAmAmAmGfCfAfAmCmCmUmCmAmGmAmAmCmAGal | 490 |
| 108 | | NAc(L96) | |
| | AS | mU*dG*mUmUdCmUdGmAmGmGmUtnUmGfCmUmUmUmUmG*m | 542 |
| | | U*mU | |
| Conjugate | SS | mC*mA*mAmAmAmGfCfAfAmCmCmUmCmAmGmAmAmCmUGal | 543 |
| 109 | | NAc(L96) | |
| | AS | mA*dG*mUmUdCmUdGmAmGmGmUtnUmGfCmUmUmUmUmG*m | 544 |
| | | U*mU | |
| Conjugate | SS | mC*mA*mAmAmAmGfCfAfAmUmCmUmCmAmGmAmAmCmAGal | 491 |
| 110 | | NAc(L96) | |
| | AS | mU*dG*mUmUdCmUdGmAmGmGmUtnUmGfCmUmUmUmUmG*m | 542 |
| | | U*mU | |
| Conjugate | SS | mC*mA*mAmAmAmGfCfAfAmUmCmUmCmAmGmAmAmCmUGal | 545 |
| 111 | | NAc(L96) | |
| | AS | mA*dG*mUmUdCmUdGmAmGmGmUtnUmGfCmUmUmUmUmG*m | 544 |
| | | U*mU | |
| Conjugate | SS | mG*mA*mAmCmAmAfAmAfGfCfAmAmCmCmUmCmAmGmAmA | 546 |
| 112 | | mUGalNAc(L96) | |
| | AS | mA*fU*mUmCmUfGmAfGfGmUmUmGmCfUmUfUmUmGmUmUm | 547 |
| | | C*mU*mU | |
| Conjugate | SS | mG*mA*mAmCmAmAfAmAfGfCfAmAmCmCmUmCmAmGmAmA | 546 |
| 113 | | mUGalNAc(L96) | |
| | AS | mA*fU*mUmCmUfGmAmGmGmUmUmGmCfUmUfUmUmGmUmU | 548 |
| | | mC*mU*mU | |
| Conjugate | SS | mG*mA*mAmCmGmAmAmAfGfUfAmAmCmCmUmCmAmGmAm | 549 |
| 114 | | AmUGalNAc(L96) | |
| | AS | mA*fU*mUmCmUfGmAmGmGmUmUmGmCfUmUfUmCmGmUmU | 550 |
| | | mC*mU*mU | |
| Conjugate | SS | mA*mG*mUmUmCmUfUmGfGfAfAmUmAmAmAmAmGmCmAmA | 422 |
| 115 | | mUGalNAc(L96) | |
| | AS | eVPmA*fU*mUmGgnCfUmUmUmUfAmUmUmCfCmAfAmGmAmA | 551 |
| | | mCmU*mC*mU | |
| Conjugate | SS | mA*mG*mUmUmCmUfUmGfGfAfAmUmAmAmAmAmGmCmAmA | 422 |
| 116 | | mUGalNAc(L96) | |
| | AS | eVPmA*fU*mUmGmCgnTmUmUmUfAmUmUmCfCmAfAmGmAmA | 552 |
| | | mCmU*mC*mU | |
| Conjugate | SS | mA*mG*mUmUmCmUfUmGfGfAfAmUmAmAmAmAmGmCmAmA | 422 |
| 117 | | mUGalNAc(L96) | |
| | AS | eVPmA*fU*mUmGmCfUgnTmUmUfAmUmUmCfCmAfAmGmAmA | 553 |
| | | mCmU*mC*mU | |
| Conjugate | SS | mA*mG*mUmUmCmUfUmGfGfAfAmUmAmAmAmAmGmCmAmA | 422 |
| 118 | | mUGalNAc(L96) | |
| | AS | eVPmA*fU*mUmGmCfUmUgnTmUfAmUmUmCfCmAfAmGmAmA | 554 |
| | | mCmU*mC*mU | |
| Conjugate | SS | mA*mG*mUmUmCmUfUmGfGfAfAmUmAmAmAmAmGmCmAmA | 422 |
| 119 | | mUGalNAc(L96) | |
| | AS | eVPmA*fU*mUmGmIfUmUmUmUfAmUmUmCfCmAfAmGmAmAm | 555 |
| | | CmU*mC*mU | |
| Conjugate | SS | mA*mG*mUmUmCmUfUmGfGfAfAmUmAmAmAmAmGmCmAmA | 422 |
| 120 | | mUGalNAc(L96) | |
| | AS | eVPmA*fU*mUmGmCmImUmUmUfAmUmUmCfCmAfAmGmAmA | 556 |
| | | mCmU*mC*mU | |
| Conjugate | SS | mA*mG*mUmUmCmUfUmGfGfAfAmUmAmAmAmAmGmCmAmA | 422 |
| 121 | | mUGalNAc(L96) | |
| | AS | eVPmA*fU*mUmGmCfUmImUmUfAmUmUmCfCmAfAmGmAmAm | 557 |
| | | CmU*mC*mU | |
| Conjugate | SS | mA*mG*mUmUmCmUfUmGfGfAfAmUmAmAmAmAmGmCmAmA | 422 |
| 122 | | mUGalNAc(L96) | |
| | AS | eVPmA*fU*mUmGmCfUmUmImUfAmUmUmCfCmAfAmGmAmAm | 558 |
| | | CmU*mC*mU | |
| Conjugate | SS | mCmAmAmAmAmGfCfAfAmUmCmUmCmAmGmAmAmCmAGalN | 559 |
| 123 | | Ac(L96) | |
| | AS | eVPmU*fG*mUmUgnCfUmGmAmGmGmUmUmGfCmUfUmUmUm | 560 |
| | | G*mU*mU | |
| Conjugate | SS | mCmAmAmAmAmGfCfAfAmUmCmUmCmAmGmAmAmCmAGalN | 559 |
| 124 | | Ac(L96) | |
| | AS | e VPmU*fG*mUmUmCgnTmGmAmGmGmUmUmGfCmUfUmUmUm | 561 |
| | | G*mU*mU | |
| Conjugate | SS | mCmAmAmAmAmGfCfAfAmUmCmUmCmAmGmAmAmCmAGalN | 559 |
| 125 | | Ac(L96) | |
| | AS | eVPmU*fG*mUmUmCfUgnGmAmGmGmUmUmGfCmUfUmUmUm | 562 |
| | | G*mU*mU | |
| Conjugate | SS | mCmAmAmAmAmGfCfAfAmUmCmUmCmAmGmAmAmCmAGalN | 559 |
| 126 | | Ac(L96) | |
| | AS | eVPmU*fG*mUmUmCfUmGgnAmGmGmUmUmGfCmUfUmUmUm | 563 |
| | | G*mU*mU | |
| Conjugate | SS | mCmAmAmAmAmGfCfAfAmUmCmUmCmAmGmAmAmCmAGalN | 559 |
| 127 | | Ac(L96) | |
| | AS | eVPmU*fG*mUmUmCmImGmAmGmGmUmUmGfCmUfUmUmUmG | 564 |
| | | *mU*mU | |
| Conjugate | SS | mCmAmAmAmAmGfCfAfAmUmCmUmCmAmGmAmAmCmAGalN | 559 |
| 128 | | Ac(L96) | |
| | AS | eVPmU*fG*mUmUmCfUmImAmGmGmUmUmGfCmUfUmUmUmG | 565 |
| | | *mU*mU | |
| Conjugate | SS | mCmAmAmAmAmGfCfAfAmUmCmUmCmAmGmAmAmCmAGalN | 559 |
| 129 | | Ac(L96) | |
| | AS | eVPmU*fG*mUmUmCfUmGmImGmGmUmUmGfCmUfUmUmUmG | 566 |
| | | *mU*mU | |
| Conjugate | SS | mA*mG*mUmUmCmUfUmGfGfAfAmUmAmAmAmAmGmCmAmA | 422 |
| 130 | | mUGalNAc(L96) | |
| | AS | eVPmA*fU*mUmGmCfUmUmUmUmAmUmUmCfCmAfAmGmAmA | 567 |
| | | mCmU*mU*mU | |
| Conjugate | SS | mA*mG*mUmUmCmUfUmGfGfAfAmUmAmAmAmAmGmCmAmA | 422 |
| 131 | | mUGalNAc(L96) | |
| | AS | eVPmA*fU*mUmGmCfUmUfUfUmAmUmUmCfCmAfAmGmAmAm | 568 |
| | | CmU*mU*mU | |
| Conjugate | SS | mA*mG*mUmUmCmUfUmGfGfAfAmUmAmAmAmAmGmCmAmA | 422 |
| 132 | | mUGalNAc(L96) | |
| | AS | eVPmA*fU*mUmGfCfUmUmUmUmAmUmUmCfCmAfAmGmAmA | 569 |
| | | mCmU*mU*mU | |
| Conjugate | SS | mA*mG*mUmUmCmUfUmGfGfAfAmUmAmAmAmAmGmCmAmA | 422 |
| 133 | | mUGalNAc(L96) | |
| | AS | eVPmA*fU*mUmGmCfUmUmUmUfAmUmUmCfCmAfAmGmAmA | 570 |
| | | mCmU*mU*mU | |
| Conjugate | SS | mCmAmAmAmAmGfCfAfAmUmCmUmCmAmGmAmAmCmAGalN | 559 |
| 134 | | Ac(L96) | |
| | AS | eVPmU*fG*mUmUmCfUmGmAmGmGmUmUmGfCmUfUmUmUmG | 571 |
| | | *mU*mU | |
| Conjugate | SS | mA*mG*mAmGmUmUfCfUfUmGmGmAmAmUmAmAmAmAmA*G | 572 |
| 135 | | alNAc(L96) | |
| | AS | mU*fU*mUmUmUfAmUmUmCmCmAmAmGfAmAfCmUmCmU*m | 573 |
| | | G*mU | |
| Conjugate | SS | mA*mG*mAmGfUmUfCfUfUmGmGmAmAmUmAmAmAmAmA*Ga | 574 |
| 136 | | lNAc(L96) | |
| | AS | mU*fU*mUmUmUfAmUmUmCmCmAmAmGfAmAfCmUmCmU*m | 573 |
| | | G*mU | |
| Conjugate | SS | mA*mG*mAmGfUmUfCfUfUmGmGmAmAmUmAmAmAmAmA*Ga | 574 |
| 137 | | lNAc(L96) | |
| | AS | mU*fU*mUmUmUfAmUfUfCmCmAmAmGfAmAfCmUmCmU*mG* | 575 |
| | | mU | |
| Conjugate | SS | mC*mA*mGmAmGmUmUfCfUfUmGmGmAmAmUmAmAmAmAm | 576 |
| 138 | | A*GalNAc(L96) | |
| | AS | mU*fU*mUmUmUfAmUmUmCmCmAmAmGfAmAfCmUmCmUmG | 577 |
| | | *mU*mG | |
| Conjugate | SS | mC*mA*mGmAmGfUmUfCfUfUmGmGmAmAmUmAmAmAmAmA | 578 |
| 139 | | *GalNAc(L96) | |
| | AS | mU*fU*mUmUmUfAmUmUmCmCmAmAmGfAmAfCmUmCmUmG | 577 |
| | | *mU*mG | |
| Conjugate | SS | mC*mA*mGmAmGfUmUfCfUfUmGmGmAmAmUmAmAmAmAmA | 578 |
| 140 | | *GalNAc(L96) | |
| | AS | mU*fU*mUmUmUfAmUfUfCmCmAmAmGfAmAfCmUmCmUmG*m | 579 |
| | | U*mG | |
| Conjugate | SS | mA*mC*mAmGmAmGmUmUfCfUfUmGmGmAmAmUmAmAmAm | 580 |
| 141 | | AmAGalNAc(L96) | |
| | AS | mU*fU*mUmUmUfAmUmUmCmCmAmAmGfAmAfCmUmCmUmG | 581 |
| | | mU*mG*mA | |
| Conjugate | SS | mA*mC*mAmGmAmGfUmUfCfUfUmGmGmAmAmUmAmAmAmA | 582 |
| 142 | | mAGalNAc(L96) | |
| | AS | mU*fU*mUmUmUfAmUmUmCmCmAmAmGfAmAfCmUmCmUmG | 581 |
| | | mU*mG*mA | |
| Conjugate | SS | mA*mC*mAmGmAmGfUmUfCfUfUmGmGmAmAmUmAmAmAmA | 582 |
| 143 | | mAGalNAc(L96) | |
| | AS | mU*fU*mUmUmUfAmUfUfCmCmAmAmGfAmAfCmUmCmUmGm | 583 |
| | | U*mG*mA | |
| Conjugate | SS | mA*mG*mAmGmUmUfCfUfUmGmGmAmAmUmAmAmAmAmA*G | 572 |
| 144 | | alNAc(L96) | |
| | AS | eVPmU*fU*mUmUmUfAmUmUmCmCmAmAmGfAmAfCmUmCmU | 584 |
| | | *mG*mU | |
| Conjugate | SS | mA*mG*mAmGfUmUfCfUfUmGmGmAmAmUmAmAmAmAmA*Ga | 574 |
| 145 | | lNAc(L96) | |
| | AS | eVPmU*fU*mUmUmUfAmUmUmCmCmAmAmGfAmAfCmUmCmU | 584 |
| | | *mG*mU | |
| Conjugate | SS | mA*mG*mAmGfUmUfCfUfUmGmGmAmAmUmAmAmAmAmA*Ga | 574 |
| 146 | | lNAc(L96) | |
| | AS | eVPmU*fU*mUmUmUfAmUfUfCmCmAmAmGfAmAfCmUmCmU* | 585 |
| | | mG*mU | |
| Conjugate | SS | mC*mA*mGmAmGmUmUfCfUfUmGmGmAmAmUmAmAmAmAm | 576 |
| 147 | | A*GalNAc(L96) | |
| | AS | eVPmU*fU*mUmUmUfAmUmUmCmCmAmAmGfAmAfCmUmCmU | 586 |
| | | mG*mU*mG | |
| Conjugate | SS | mC*mA*mGmAmGfUmUfCfUfUmGmGmAmAmUmAmAmAmAmA | 578 |
| 148 | | *GalNAc(L96) | |
| | AS | eVPmU*fU*mUmUmUfAmUmUmCmCmAmAmGfAmAfCmUmCmU | 586 |
| | | mG*mU*mG | |
| Conjugate | SS | mC*mA*mGmAmGfUmUfCfUfUmGmGmAmAmUmAmAmAmAmA | 578 |
| 149 | | *GalNAc(L96) | |
| | AS | eVPmU*fU*mUmUmUfAmUfUfCmCmAmAmGfAmAfCmUmCmUm | 587 |
| | | G*mU*mG | |
| Conjugate | SS | mA*mC*mAmGmAmGmUmUfCfUfUmGmGmAmAmUmAmAmAm | 580 |
| 150 | | AmAGalNAc(L96) | |
| | AS | eVPmU*fU*mUmUmUfAmUmUmCmCmAmAmGfAmAfCmUmCmU | 588 |
| | | mGmU*mG*mA | |
| Conjugate | SS | mA*mC*mAmGmAmGfUmUfCfUfUmGmGmAmAmUmAmAmAmA | 582 |
| 151 | | mAGalNAc(L96) | |
| | AS | eVPmU*fU*mUmUmUfAmUmUmCmCmAmAmGfAmAfCmUmCmU | 588 |
| | | mGmU*mG*mA | |
| Conjugate | SS | mA*mC*mAmGmAmGfUmUfCfUfUmGmGmAmAmUmAmAmAmA | 582 |
| 152 | | mAGalNAc(L96) | |
| | AS | eVPmU*fU*mUmUmUfAmUfUfCmCmAmAmGfAmAfCmUmCmUm | 589 |
| | | GmU*mG*mA | |
| Conjugate | SS | mU*mC*mUmUmGmGfAfAfUmAmAmAmAmGmCmAmAmCmU*G | 590 |
| 153 | | alNAc(L96) | |
| | AS | mA*fG*mUmUmGfCmUmUmUmUmAmUmUfCmCfAmAmGmA*m | 591 |
| | | A*mC | |
| Conjugate | SS | mU*mC*mUmUfGmGfAfAfUmAmAmAmAmGmCmAmAmCmU*Ga | 592 |
| 154 | | lNAc(L96) | |
| | AS | mA*fG*mUmUmGfCmUmUmUmUmAmUmUfCmCfAmAmGmA*m | 591 |
| | | A*mC | |
| Conjugate | SS | mU*mC*mUmUfGmGfAfAfUmAmAmAmAmGmCmAmAmCmU*Ga | 592 |
| 155 | | lNAc(L96) | |
| | AS | mA*fG*mUmUmGfCmUfUfUmUmAmUmUfCmCfAmAmGmA*mA* | 593 |
| | | mC | |
| Conjugate | SS | mU*mU*mCmUmUmGmGfAfAfUmAmAmAmAmGmCmAmAmCm | 594 |
| 156 | | U*GalNAc(L96) | |
| | AS | mA*fG*mUmUmGfCmUmUmUmUmAmUmUfCmCfAmAmGmAmA | 595 |
| | | *mC*mU | |
| Conjugate | SS | mU*mU*mCmUmUfGmGfAfAfUmAmAmAmAmGmCmAmAmCmU | 596 |
| 157 | | *GalNAc(L96) | |
| | AS | mA*fG*mUmUmGfCmUmUmUmUmAmUmUfCmCfAmAmGmAmA | 595 |
| | | *mC*mU | |
| Conjugate | SS | mU*mU*mCmUmUfGmGfAfAfUmAmAmAmAmGmCmAmAmCmU | 596 |
| 158 | | *GalNAc(L96) | |
| | AS | mA*fG*mUmUmGfCmUfUfUmUmAmUmUfCmCfAmAmGmAmA* | 597 |
| | | mC*mU | |
| Conjugate | SS | mG*mU*mUmCmUmUmGmGfAfAfUmAmAmAmAmGmCmAmAm | 598 |
| 159 | | CmUGalNAc(L96) | |
| | AS | mA*fG*mUmUmGfCmUmUmUmUmAmUmUfCmCfAmAmGmAmA | 425 |
| | | mC*mU*mC | |
| Conjugate | SS | mG*mU*mUmCmUmUfGmGfAfAfUmAmAmAmAmGmCmAmAmC | 424 |
| 160 | | mUGalNAc(L96) | |
| | AS | mA*fG*mUmUmGfCmUmUmUmUmAmUmUfCmCfAmAmGmAmA | 425 |
| | | mC*mU*mC | |
| Conjugate | SS | mG*mU*mUmCmUmUfGmGfAfAfUmAmAmAmAmGmCmAmAmC | 424 |
| 161 | | mUGalNAc(L96) | |
| | AS | mA*fG*mUmUmGfCmUfUfUmUmAmUmUfCmCfAmAmGmAmAm | 431 |
| | | C*mU*mC | |
| Conjugate | SS | mU*mC*mUmUmGmGfAfAfUmAmAmAmAmGmCmAmAmCmU*G | 590 |
| 162 | | alNAc(L96) | |
| | AS | eVPmA*fG*mUmUmGfCmUmUmUmUmAmUmUfCmCfAmAmGmA | 599 |
| | | *mA*mC | |
| Conjugate | SS | mU*mC*mUmUfGmGfAfAfUmAmAmAmAmGmCmAmAmCmU*Ga | 592 |
| 163 | | lNAc(L96) | |
| | AS | eVPmA*fG*mUmUmGfCmUmUmUmUmAmUmUfCmCfAmAmGmA | 599 |
| | | *mA*mC | |
| Conjugate | SS | mU*mC*mUmUfGmGfAfAfUmAmAmAmAmGmCmAmAmCmU*Ga | 592 |
| 164 | | lNAc(L96) | |
| | AS | eVPmA*fG*mUmUmGfCmUfUfUmUmAmUmUfCmCfAmAmGmA* | 600 |
| | | mA*mC | |
| Conjugate | SS | mU*mU*mCmUmUmGmGfAfAfUmAmAmAmAmGmCmAmAmCm | 594 |
| 165 | | U*GalNAc(L96) | |
| | AS | eVPmA*fG*mUmUmGfCmUmUmUmUmAmUmUfCmCfAmAmGmA | 601 |
| | | mA*mC*mU | |
| Conjugate | SS | mU*mU*mCmUmUfGmGfAfAfUmAmAmAmAmGmCmAmAmCmU | 596 |
| 166 | | *GalNAc(L96) | |
| | AS | eVPmA*fG*mUmUmGfCmUmUmUmUmAmUmUfCmCfAmAmGmA | 601 |
| | | mA*mC*mU | |
| Conjugate | SS | mU*mU*mCmUmUfGmGfAfAfUmAmAmAmAmGmCmAmAmCmU | 596 |
| 167 | | *GalNAc(L96) | |
| | AS | eVPmA*fG*mUmUmGfCmUfUfUmUmAmUmUfCmCfAmAmGmAm | 602 |
| | | A*mC*mU | |
| Conjugate | SS | mG*mU*mUmCmUmUmGmGfAfAfUmAmAmAmAmGmCmAmAm | 598 |
| 168 | | CmUGalNAc(L96) | |
| | AS | eVPmA*fG*mUmUmGfCmUmUmUmUmAmUmUfCmCfAmAmGmA | 603 |
| | | mAmC*mU*mC | |
| Conjugate | SS | mG*mU*mUmCmUmUfGmGfAfAfUmAmAmAmAmGmCmAmAmC | 424 |
| 169 | | mUGalNAc(L96) | |
| | AS | eVPmA*fG*mUmUmGfCmUmUmUmUmAmUmUfCmCfAmAmGmA | 603 |
| | | mAmC*mU*mC | |
| Conjugate | SS | mG*mU*mUmCmUmUfGmGfAfAfUmAmAmAmAmGmCmAmAmC | 424 |
| 170 | | mUGalNAc(L96) | |
| | AS | eVPmA*fG*mUmUmGfCmUfUfUmUmAmUmUfCmCfAmAmGmAm | 604 |
| | | AmC*mU*mC | |
| Conjugate | SS | mC*mU*mUmGmGmAfAfUfAmAmAmAmGmCmAmAmCmCmU*G | 605 |
| 171 | | alNAc(L96) | |
| | AS | mA*fG*mGmUmUfGmCmUmUmUmUmAmUfUmCfCmAmAmG*m | 606 |
| | | A*mA | |
| Conjugate | SS | mC*mU*mUmGfGmAfAfUfAmAmAmAmGmCmAmAmCmCmU*Ga | 607 |
| 172 | | lNAc(L96) | |
| | AS | mA*fG*mGmUmUfGmCmUmUmUmUmAmUfUmCfCmAmAmG*m | 606 |
| | | A*mA | |
| Conjugate | SS | mC*mU*mUmGfGmAfAfUfAmAmAmAmGmCmAmAmCmCmU*Ga | 607 |
| 173 | | lNAc(L96) | |
| | AS | mA*fG*mGmUmUfGmCfUfUmUmUmAmUfUmCfCmAmAmG*mA* | 608 |
| | | mA | |
| Conjugate | SS | mU*mC*mUmUmGmGmAfAfUfAmAmAmAmGmCmAmAmCmCm | 609 |
| 174 | | U*GalNAc(L96) | |
| | AS | mA*fG*mGmUmUfGmCmUmUmUmUmAmUfUmCfCmAmAmGmA | 610 |
| | | *mA*mC | |
| Conjugate | SS | mU*mC*mUmUmGfGmAfAfUfAmAmAmAmGmCmAmAmCmCmU | 611 |
| 175 | | *GalNAc(L96) | |
| | AS | mA*fG*mGmUmUfGmCmUmUmUmUmAmUfUmCfCmAmAmGmA | 610 |
| | | *mA*mC | |
| Conjugate | SS | mU*mC*mUmUmGfGmAfAfUfAmAmAmAmGmCmAmAmCmCmU | 611 |
| 176 | | *GalNAc(L96) | |
| | AS | mA*fG*mGmUmUfGmCfUfUmUmUmAmUfUmCfCmAmAmGmA* | 612 |
| | | mA*mC | |
| Conjugate | SS | mU*mU*mCmUmUmGmGmAfAfUfAmAmAmAmGmCmAmAmCm | 613 |
| 177 | | CmUGalNAc(L96) | |
| | AS | mA*fG*mGmUmUfGmCmUmUmUmUmAmUfUmCfCmAmAmGmA | 614 |
| | | mA*mC*mU | |
| Conjugate | SS | mU*mU*mCmUmUmGfGmAfAfUfAmAmAmAmGmCmAmAmCmC | 615 |
| 178 | | mUGalNAc(L96) | |
| | AS | mA*fG*mGmUmUfGmCmUmUmUmUmAmUfUmCfCmAmAmGmA | 614 |
| | | mA*mC*mU | |
| Conjugate | SS | mU*mU*mCmUmUmGfGmAfAfUfAmAmAmAmGmCmAmAmCmC | 615 |
| 179 | | mUGalNAc(L96) | |
| | AS | mA*fG*mGmUmUfGmCfUfUmUmUmAmUfUmCfCmAmAmGmAm | 616 |
| | | A*mC*mU | |
| Conjugate | SS | mC*mU*mUmGmGmAfAfUfAmAmAmAmGmCmAmAmCmCmU*G | 605 |
| 180 | | alNAc(L96) | |
| | AS | eVPmA*fG*mGmUmUfGmCmUmUmUmUmAmUfUmCfCmAmAmG | 617 |
| | | *mA*mA | |
| Conjugate | SS | mC*mUmUmGfGmAfAfUfAmAmAmAmGmCmAmAmCmCmU*Ga | 607 |
| 181 | | lNAc(L96) | |
| | AS | eVPmA*fG*mGmUmUfGmCmUmUmUmUmAmUfUmCfCmAmAmG | 617 |
| | | *mA*mA | |
| Conjugate | SS | mC*mU*mUmGfGmAfAfUfAmAmAmAmGmCmAmAmCmCmU*Ga | 607 |
| 182 | | lNAc(L96) | |
| | AS | eVPmA*fG*mGmUmUfGmCfUfUmUmUmAmUfUmCfCmAmAmG* | 618 |
| | | mA*mA | |
| Conjugate | SS | mU*mC*mUmUmGmGmAfAfUfAmAmAmAmGmCmAmAmCmCm | 609 |
| 183 | | U*GalNAc(L96) | |
| | AS | eVPmA*fG*mGmUmUfGmCmUmUmUmUmAmUfUmCfCmAmAmG | 619 |
| | | mA*mA*mC | |
| Conjugate | SS | mU*mC*mUmUmGfGmAfAfUfAmAmAmAmGmCmAmAmCmCmU | 611 |
| 184 | | *GalNAc(L96) | |
| | AS | eVPmA*fG*mGmUmUfGmCmUmUmUmUmAmUfUmCfCmAmAmG | 619 |
| | | mA*mA*mC | |
| Conjugate | SS | mU*mC*mUmUmGfGmAfAfUfAmAmAmAmGmCmAmAmCmCmU | 611 |
| 185 | | *GalNAc(L96) | |
| | AS | eVPmA*fG*mGmUmUfGmCfUfUmUmUmAmUfUmCfCmAmAmGm | 620 |
| | | A*mA*mC | |
| Conjugate | SS | mU*mU*mCmUmUmGmGmAfAfUfAmAmAmAmGmCmAmAmCm | 613 |
| 186 | | CmUGalNAc(L96) | |
| | AS | eVPmA*fG*mGmUmUfGmCmUmUmUmUmAmUfUmCfCmAmAmG | 621 |
| | | mAmA*mC*mU | |
| Conjugate | SS | mU*mU*mCmUmUmGfGmAfAfUfAmAmAmAmGmCmAmAmCmC | 615 |
| 187 | | mUGalNAc(L96) | |
| | AS | eVPmA*fG*mGmUmUfGmCmUmUmUmUmAmUfUmCfCmAmAmG | 621 |
| | | mAmA*mC*mU | |
| Conjugate | SS | mU*mU*mCmUmUmGfGmAfAfUfAmAmAmAmGmCmAmAmCmC | 615 |
| 188 | | mUGalNAc(L96) | |
| | AS | eVPmA*fG*mGmUmUfGmCfUfUmUmUmAmUfUmCfCmAmAmGm | 622 |
| | | AmA*mC*mU | |
| Conjugate | SS | mU*mU*mGmGmAmAfUfAfAmAmAmGmCmAmAmCmCmUmU*G | 623 |
| 189 | | alNAc(L96) | |
| | AS | mA*fA*mGmGmUfUmGmCmUmUmUmUmAfUmUfCmCmAmA*m | 624 |
| | | G*mA | |
| Conjugate | SS | mU*mU*mGmGfAmAfUfAfAmAmAmGmCmAmAmCmCmUmU*Ga | 625 |
| 190 | | lNAc(L96) | |
| | AS | mA*fA*mGmGmUfUmGmCmUmUmUmUmAfUmUfCmCmAmA*m | 624 |
| | | G*mA | |
| Conjugate | SS | mU*mU*mGmGfAmAfUfAfAmAmAmGmCmAmAmCmCmUmU*Ga | 625 |
| 191 | | lNAc(L96) | |
| | AS | mA*fA*mGmGmUfUmGfCfUmUmUmUmAfUmUfCmCmAmA*mG* | 626 |
| | | mA | |
| Conjugate | SS | mC*mU*mUmGmGmAmAfUfAfAmAmAmGmCmAmAmCmCmUm | 627 |
| 192 | | U*GalNAc(L96) | |
| | AS | mA*fA*mGmGmUfUmGmCmUmUmUmUmAfUmUfCmCmAmAmG | 628 |
| | | *mA*mA | |
| Conjugate | SS | mC*mU*mUmGmGfAmAfUfAfAmAmAmGmCmAmAmCmCmUmU | 629 |
| 193 | | *GalNAc(L96) | |
| | AS | mA*fA*mGmGmUfUmGmCmUmUmUmUmAfUmUfCmCmAmAmG | 628 |
| | | *mA*mA | |
| Conjugate | SS | mC*mU*mUmGmGfAmAfUfAfAmAmAmGmCmAmAmCmCmUmU | 629 |
| 194 | | *GalNAc(L96) | |
| | AS | mA*fA*mGmGmUfUmGfCfUmUmUmUmAfUmUfCmCmAmAmG* | 630 |
| | | mA*mA | |
| Conjugate | SS | mU*mC*mUmUmGmGmAmAfUfAfAmAmAmGmCmAmAmCmCm | 631 |
| 195 | | UmUGalNAc(L96) | |
| | AS | mA*fA*mGmGmUfUmGmCmUmUmUmUmAfUmUfCmCmAmAmG | 427 |
| | | mA*mA*mC | |
| Conjugate | SS | mU*mC*mUmUmGmGfAmAfUfAfAmAmAmGmCmAmAmCmCmU | 426 |
| 196 | | mUGalNAc(L96) | |
| | AS | mA*fA*mGmGmUfUmGmCmUmUmUmUmAfUmUfCmCmAmAmG | 427 |
| | | mA*mA*mC | |
| Conjugate | SS | mU*mC*mUmUmGmGfAmAfUfAfAmAmAmGmCmAmAmCmCmU | 426 |
| 197 | | mUGalNAc(L96) | |
| | AS | mA*fA*mGmGmUfUmGfCfUmUmUmUmAfUmUfCmCmAmAmGm | 432 |
| | | A*mA*mC | |
| Conjugate | SS | mU*mU*mGmGmAmAfUfAfAmAmAmGmCmAmAmCmCmUmU*G | 623 |
| 198 | | alNAc(L96) | |
| | AS | eVPmA*fA*mGmGmUfUmGmCmUmUmUmUmAfUmUfCmCmAmA | 632 |
| | | *mG*mA | |
| Conjugate | SS | mU*mU*mGmGfAmAfUfAfAmAmAmGmCmAmAmCmCmUmU*Ga | 625 |
| 199 | | lNAc(L96) | |
| | AS | eVPmA*fA*mGmGmUfUmGmCmUmUmUmUmAfUmUfCmCmAmA | 632 |
| | | *mG*mA | |
| Conjugate | SS | mU*mU*mGmGfAmAfUfAfAmAmAmGmCmAmAmCmCmUmU*Ga | 625 |
| 200 | | lNAc(L96) | |
| | AS | eVPmA*fA*mGmGmUfUmGfCfUmUmUmUmAfUmUfCmCmAmA* | 633 |
| | | mG*mA | |
| Conjugate | SS | mC*mU*mUmGmGmAmAfUfAfAmAmAmGmCmAmAmCmCmUm | 627 |
| 201 | | U*GalNAc(L96) | |
| | AS | eVPmA*fA*mGmGmUfUmGmCmUmUmUmUmAfUmUfCmCmAmA | 634 |
| | | mG*mA*mA | |
| Conjugate | SS | mC*mU*mUmGmGfAmAfUfAfAmAmAmGmCmAmAmCmCmUmU | 629 |
| 202 | | *GalNAc(L96) | |
| | AS | eVPmA*fA*mGmGmUfUmGmCmUmUmUmUmAfUmUfCmCmAmA | 634 |
| | | mG*mA*mA | |
| Conjugate | SS | mC*mU*mUmGmGfAmAfUfAfAmAmAmGmCmAmAmCmCmUmU | 629 |
| 203 | | *GalNAc(L96) | |
| | AS | eVPmA*fA*mGmGmUfUmGfCfUmUmUmUmAfUmUfCmCmAmAm | 635 |
| | | G*mA*mA | |
| Conjugate | SS | mU*mC*mUmUmGmGmAmAfUfAfAmAmAmGmCmAmAmCmCm | 631 |
| 204 | | UmUGalNAc(L96) | |
| | AS | eVPmA*fA*mGmGmUfUmGmCmUmUmUmUmAfUmUfCmCmAmA | 636 |
| | | mGmA*mA*mC | |
| Conjugate | SS | mU*mC*mUmUmGmGfAmAfUfAfAmAmAmGmCmAmAmCmCmU | 426 |
| 205 | | mUGalNAc(L96) | |
| | AS | eVPmA*fA*mGmGmUfUmGmCmUmUmUmUmAfUmUfCmCmAmA | 636 |
| | | mGmA*mA*mC | |
| Conjugate | SS | mU*mC*mUmUmGmGfAmAfUfAfAmAmAmGmCmAmAmCmCmU | 426 |
| 206 | | mUGalNAc(L96) | |
| | AS | eVPmA*fA*mGmGmUfUmGfCfUmUmUmUmAfUmUfCmCmAmAm | 637 |
| | | GmA*mA*mC | |
| Conjugate | SS | mA*mA*mUmAmAmAfAfGfCmAmAmCmCmUmCmAmGmAmA*G | 638 |
| 207 | | alNAc(L96) | |
| | AS | mU*fU*mCmUmGfAmGmGmUmUmGmCmUfUmUfUmAmUmU*m | 639 |
| | | C*mC | |
| Conjugate | SS | mA*mA*mUmAfAmAfAfGfCmAmAmCmCmUmCmAmGmAmA*Ga | 640 |
| 208 | | lNAc(L96) | |
| | AS | mU*fU*mCmUmGfAmGmGmUmUmGmCmUfUmUfUmAmUmU*m | 639 |
| | | C*mC | |
| Conjugate | SS | mA*mA*mUmAfAmAfAfGfCmAmAmCmCmUmCmAmGmAmA*Ga | 640 |
| 209 | | lNAc(L96) | |
| | AS | mU*fU*mCmUmGfAmGfGfUmUmGmCmUfUmUfUmAmUmU*mC* | 641 |
| | | mC | |
| Conjugate | SS | mG*mA*mAmUmAmAmAfAfGfCmAmAmCmCmUmCmAmGmAm | 642 |
| 210 | | A*GalNAc(L96) | |
| | AS | mU*fU*mCmUmGfAmGmGmUmUmGmCmUfUmUfUmAmUmUmC | 643 |
| | | *mC*mA | |
| Conjugate | SS | mG*mA*mAmUmAfAmAfAfGfCmAmAmCmCmUmCmAmGmAmA | 644 |
| 211 | | *GalNAc(L96) | |
| | AS | mU*fU*mCmUmGfAmGmGmUmUmGmCmUfUmUfUmAmUmUmC | 643 |
| | | *mC*mA | |
| Conjugate | SS | mG*mA*mAmUmAfAmAfAfGfCmAmAmCmCmUmCmAmGmAmA | 644 |
| 212 | | *GalNAc(L96) | |
| | AS | mU*fU*mCmUmGfAmGfGfUmUmGmCmUfUmUfUmAmUmUmC* | 645 |
| | | mC*mA | |
| Conjugate | SS | mG*mG*mAmAmUmAmAmAfAfGfCmAmAmCmCmUmCmAmGm | 646 |
| 213 | | AmAGalNAc(L96) | |
| | AS | mU*fU*mCmUmGfAmGmGmUmUmGmCmUfUmUfUmAmUmUmC | 429 |
| | | mC*mA*mA | |
| Conjugate | SS | mG*mG*mAmAmUmAfAmAfAfGfCmAmAmCmCmUmCmAmGmA | 428 |
| 214 | | mAGalNAc(L96) | |
| | AS | mU*fU*mCmUmGfAmGmGmUmUmGmCmUfUmUfUmAmUmUmC | 429 |
| | | mC*mA*mA | |
| Conjugate | SS | mG*mG*mAmAmUmAfAmAfAfGfCmAmAmCmCmUmCmAmGmA | 428 |
| 215 | | mAGalNAc(L96) | |
| | AS | mU*fU*mCmUmGfAmGfGfUmUmGmCmUfUmUfUmAmUmUmCm | 433 |
| | | C*mA*mA | |
| Conjugate | SS | mA*mA*mUmAmAmAfAfGfCmAmAmCmCmUmCmAmGmAmA*G | 638 |
| 216 | | alNAc(L96) | |
| | AS | eVPmU*fU*mCmUmGfAmGmGmUmUmGmCmUfUmUfUmAmUmU | 647 |
| | | *mC*mC | |
| Conjugate | SS | mA*mA*mUmAfAmAfAfGfCmAmAmCmCmUmCmAmGmAmA*Ga | 640 |
| 217 | | lNAc(L96) | |
| | AS | eVPmU*fU*mCmUmGfAmGmGmUmUmGmCmUfUmUfUmAmUmU | 647 |
| | | *mC*mC | |
| Conjugate | SS | mA*mA*mUmAfAmAfAfGfCmAmAmCmCmUmCmAmGmAmA*Ga | 640 |
| 218 | | lNAc(L96) | |
| | AS | eVPmU*fU*mCmUmGfAmGfGfUmUmGmCmUfUmUfUmAmUmU* | 648 |
| | | mC*mC | |
| Conjugate | SS | mG*mA*mAmUmAmAmAfAfGfCmAmAmCmCmUmCmAmGmAm | 642 |
| 219 | | A*GalNAc(L96) | |
| | AS | eVPmU*fU*mCmUmGfAmGmGmUmUmGmCmUfUmUfUmAmUmU | 649 |
| | | mC*mC*mA | |
| Conjugate | SS | mG*mA*mAmUmAfAmAfAfGfCmAmAmCmCmUmCmAmGmAmA | 644 |
| 220 | | *GalNAc(L96) | |
| | AS | eVPmU*fU*mCmUmGfAmGmGmUmUmGmCmUfUmUfUmAmUmU | 649 |
| | | mC*mC*mA | |
| Conjugate | SS | mG*mA*mAmUmAfAmAfAfGfCmAmAmCmCmUmCmAmGmAmA | 644 |
| 221 | | *GalNAc(L96) | |
| | AS | eVPmU*fU*mCmUmGfAmGfGfUmUmGmCmUfUmUfUmAmUmUm | 650 |
| | | C*mC*mA | |
| Conjugate | SS | mG*mG*mAmAmUmAmAmAfAfGfCmAmAmCmCmUmCmAmGm | 646 |
| 222 | | AmAGalNAc(L96) | |
| | AS | eVPmU*fU*mCmUmGfAmGmGmUmUmGmCmUfUmUfUmAmUmU | 651 |
| | | mCmC*mA*mA | |
| Conjugate | SS | mG*mG*mAmAmUmAfAmAfAfGfCmAmAmCmCmUmCmAmGmA | 428 |
| 223 | | mAGalNAc(L96) | |
| | AS | eVPmU*fU*mCmUmGfAmGmGmUmUmGmCmUfUmUfUmAmUmU | 651 |
| | | mCmC*mA*mA | |
| Conjugate | SS | mG*mG*mAmAmUmAfAmAfAfGfCmAmAmCmCmUmCmAmGmA | 428 |
| 224 | | mAGalNAc(L96) | |
| | AS | eVPmU*fU*mCmUmGfAmGfGfUmUmGmCmUfUmUfUmAmUmUm | 652 |
| | | CmC*mA*mA | |
| Conjugate | SS | mA*mG*mUmUmCmUfUmGfGfAfAmUmAmAmAmAmGmCmAmA | 422 |
| 225 | | mUGalNAc(L96) | |
| | AS | eVPmA*fU*mUmGgnCfUmUmUmUmAmUmUmCfCmAfAmGmAm | 653 |
| | | AmCmU*mC*mU | |
| Conjugate | SS | mA*mG*mUmUmCmUfUmGfGfAfAmUmAmAmAmAmGmCmAmA | 422 |
| 226 | | mUGalNAc(L96) | |
| | AS | eVPmA*fU*mUmGmCgnTmUmUmUmAmUmUmCfCmAfAmGmAm | 654 |
| | | AmCmU*mC*mU | |
| Conjugate | SS | mA*mG*mUmUmCmUfUmGfGfAfAmUmAmAmAmAmGmCmAmA | 422 |
| 227 | | mUGalNAc(L96) | |
| | AS | eVPmA*fU*mUmGmCfUgnTmUmUmAmUmUmCfCmAfAmGmAmA | 655 |
| | | mCmU*mC*mU | |
| Conjugate | SS | mA*mG*mUmUmCmUfUmGfGfAfAmUmAmAmAmAmGmCmAmA | 422 |
| 228 | | mUGalNAc(L96) | |
| | AS | eVPmA*fU*mUmGmCfUmUgnTmUmAmUmUmCfCmAfAmGmAmA | 656 |
| | | mCmU*mC*mU | |
| Conjugate | SS | mA*mG*mUmUmCmUfUmGfGfAfAmUmAmAmAmAmGmCmAmA | 422 |
| 229 | | mUGalNAc(L96) | |
| | AS | eVPmA*fU*mUmGmCmUmUmUmUmAmUmUmCfCmAfAmGmAm | 657 |
| | | AmCmU*mC*mU | |
| Conjugate | SS | mA*mG*mUmUmCmUfUmGfGfAfAmUmAmAmAmAmGmCmAmA | 422 |
| 230 | | mUGalNAc(L96) | |
| | AS | eVPmA*fU*mUmGmCfUmUmUmUmAmUmUmCfCmAfAmGmAmA | 658 |
| | | mCmU*ml*mU | |
| Conjugate | SS | mG*mU*mCmUfUmGfGfAfAmUmAmAmAmAmGmCmAmAmUGal | 422 |
| 231 | | NAc(L96) | |
| | AS | eVPmA*fU*mUmGmCfUmUmUmUmAmUmUmCfCmAfAmGmAmA | 460 |
| | | *mC*mU | |
| Conjugate | SS | tnG*mU*mUmCmUfUmGfGfAfAmUmAmAmAmAmGmCmAmAmU | 514 |
| 232 | | *GalNAc(L96) | |
| | AS | eVPmA*fU*mUmGmCfUmUmUmUmAmUmUmCfCmAfAmGmAmA | 459 |
| | | mC*mU*mC | |
| Conjugate | SS | mG*mU*mUmCmUfUmGfGfAfAmUmAmAmAmAmGmCmAmAmU | 516 |
| 233 | | *GalNAc(L96) | |
| | AS | eVPmA*fU*mUmGmCfUmUmUmUmAmUmUmCfCmAfAmGmAmA | 659 |
| | | mC*tnT*mC | |
| Conjugate | SS | mG*mU*mUmCmUfUmGfGfAfAmUmAmAmAmAmGmCmAmAmU | 516 |
| 234 | | *GalNAc(L96) | |
| | AS | eVPmA*fU*mUmGmCfUmUmUmUmAmUmUmCfCmAfAmGmAmA | 459 |
| | | mC*mU*mC | |
| Conjugate | SS | mG*mU*mUmCmUfUmGfGfAfAmUmAmAmAmAmGmCmAmAmU | 516 |
| 235 | | *GalNAc(L96) | |
| | AS | eVPmA*fU*mUmGmCfUmUmUmUmAmUmUmCfCmAfAmGmAmA | 660 |
| | | mC*ml*mC | |
| Conjugate | SS | tnA*mG*mUmUmCmUmUmGfGfAfAmUmAmAmAmAmGmCmAm | 502 |
| 236 | | AmUGalNAc(L96) | |
| | AS | eVPmA*fU*mUmGmCfUmUmUmUmAmUmUmCfCmAfAmGmAmA | 525 |
| | | mCmU*mC*mU | |
| Conjugate | SS | mA*mG*mUmUmCmUmUmGfGfAfAmUmAmAmAmAmGmCmAm | 464 |
| 237 | | AmUGalNAc(L96) | |
| | AS | eVPmA*fU*mUmGmCmUmUmUmUmAmUmUmCfCmAfAmGmAm | 657 |
| | | AmCmU*mC*mU | |
| Conjugate | SS | mA*mG*mUmUmCmUmUmGfGfAfAmUmAmAmAmAmGmCmAm | 464 |
| 238 | | AmUGalNAc(L96) | |
| | AS | eVPmA*fU*mUmGmCfUmUmUmUmAmUmUmCfCmAfAmGmAmA | 658 |
| | | mCmU*ml*mU | |
| Conjugate | SS | mA*mG*mUmUmCdTmUmGfGfAfAmUmAmAmAmAmGmCmAmA | 498 |
| 239 | | mUGalNAc(L96) | |
| | AS | eVPmA*fU*mUmGmCfUmUmUmUmAmUmUmCfCmAfAmGmAmA | 525 |
| | | mCmU*mC*mU | |
| Conjugate | SS | mG*mU*mUmCmUmUmGfGfAfAmUmAmAmAmAmGmCmAmAm | 519 |
| 240 | | U*GalNAc(L96) | |
| | AS | eVPmA*fU*mUmGmCfUmUmUmUmAmUmUmCfCmAfAmGmAmA | 660 |
| | | mC*mI*mC | |
| Conjugate | SS | mG*mU*mUmCmUmUmGfGfAfAmUmAmAmAmAmGmCmAmAm | 519 |
| 241 | | U*GalNAc(L96) | |
| | AS | eVPmA*fU*mUmGmCfUmUmUmUmAmUmUmCfCmAfAmGmAmA | 659 |
| | | mC*tnT*mC | |
| Conjugate | SS | mG*mU*mUmCdTmUmGfGfAfAmUmAmAmAmAmGmCmAmAmU | 513 |
| 242 | | *GalNAc(L96) | |
| | AS | eVPmA*fU*mUmGmCfUmUmUmUmAmUmUmCfCmAfAmGmAmA | 459 |
| | | mC*mU*mC | |
| Conjugate | SS | fC*mA*fGmAfGmUfUmCfUmUfGmGfAmAfUmAfAmAfA*mU*mU | 661 |
| PC c | | GalNAc(L96) | |
| (reference) | AS | mU*fU*mUfUmAfUmUfCmCfAmAfGmAfAmCfUmCfUmG*mU*mU | 662 |

Specifically, uppercase C, G, U, and A represent the base composition of a nucleotide; lowercase d represents that the nucleotide immediately to the right of the letter d is a deoxyribonucleotide; lowercase m represents that the nucleotide immediately to the right of the letter m is a 2′-methoxy nucleotide; symbol f represents that the nucleotide immediately to the right of the symbol f is a 2′-fluoro nucleotide; gn represents that the nucleotide immediately to the right of the symbol gn is a glycerol nucleotide (GNA); tn represents that the nucleotide immediately to the right of the symbol tn is a threose nucleotide (TNA); mI refers to inosine with a 2′-methoxy substitution on the ribose moiety; symbol * represents that the two adjacent nucleotides flanking the symbol * are linked via a phosphorothioate linkage; eVP represents that the nucleotide immediately to the right of the symbol eVP is a vinylphosphonate-modified nucleotide; iab represents an inverted abasic residue. GalNAc(L96) refers to conjugation with the linker-targeting ligand moiety GalNAc(L96) at this position.

The sequence of duplex 143 was chemically modified to obtain various modified siRNAs. The sequences of the modified duplexes are as follows:

TABLE 3
Sequences of modified duplexes

| | Strand | | SEQ ID |
|---|---|---|---|
| No. | type | Sequence (5′->3′) | NO |
| Modified | SS | mA*mG*mUmUmCmUmUmGfGfAfAmUmAmAmAmAmGmCmAmA | 667 |
| | | mU | |
| duplex 1 | AS | mA*fU*mUmGmCmUmUmUmUmAmUfUmCfCmAfAmGmAmAmCm | 668 |
| | | U*mC*mU | |
| Modified | SS | mA*mG*mUmUmCmUmUmGfGfAfAmUmAmAmAmAmGmCmAmA | 667 |
| | | mU | |
| duplex 2 | AS | mA*fU*mUmGfCmUmUmUmUmAmUfUmCfCmAfAmGmAmAmC | 669 |
| | | mU*mC*mU | |
| Modified | SS | mA*mG*mUmUmCmUmUmGfGfAfAmUmAmAmAmAmGmCmAmA | 667 |
| | | mU | |
| duplex 3 | AS | mA*fU*mUmGmCmUfUmUmUmAmUfUmCfCmAfAmGmAmAmC | 670 |
| | | mU*mC*mU | |
| Modified | SS | mA*mG*mUmUmCmUmUmGfGfAfAmUmAmAmAmAmGmCmAmA | 667 |
| | | mU | |
| duplex 4 | AS | mA*fU*mUmGfCmUmUfUmUmAmUfUmCfCmAfAmGmAmAmC | 671 |
| | | mU*mC*mU | |
| Modified | SS | mA*mG*mUmUmCmUfUmGfGfAfAmUmAmAmAmAmGmCmAmAm | 422 |
| | | U | |
| duplex 5 | AS | mA*fU*mUmGmCfUmUfUfUmAmUmUmCfCmAfAmGmAmAmCm | 430 |
| | | U*mC*mU | |
| Modified | SS | mA*mG*mUmUmCmUfUmGfGfAfAmUmAmAmAmAmGmCmAmAm | 672 |
| | | U | |
| duplex 6 | AS | mA*fU*mUmGmCfUmUmUmUmAmUmUmCfCmAfAmGmAmAmCm | 423 |
| | | U*mC*mU | |
| Modified | SS | mA*mG*mUmUmCmUmUmGfGfAfAmUmAmAmAmAmGmCmAmA | 667 |
| | | mU | |
| duplex 7 | AS | mA*fU*mUfGmCfUmUfUmUfAmUfUmCfCmAfAmGfAmAfCm | 673 |
| | | U*mC*mU | |
| Modified | SS | mA*mG*mUmUmCmUmUmGfGfAfAmUmAmAmAmAmGmCmAmA | 667 |
| | | mU | |
| duplex 8 | AS | mA*fU*mUmGmCmUfUmUmUmAmUmUmCfCmAfAmGmAmAmCm | 674 |
| | | U*mC*mU | |
| Modified | SS | mA*mG*mUmUmCmUmUmGfGfAfAmUmAmAmAmAmGmCmAmA | 667 |
| | | mU | |
| duplex 9 | AS | mA*fU*mUmGfCmUmUmUmUmAmUmUmCfCmAfAmGmAmAmCm | 675 |
| | | U*mC*mU | |
| Modified | SS | mA*mG*mUmUmCmUmUmGfGfAfAmUmAmAmAmAmGmCmAmA | 667 |
| | | mU | |
| duplex 10 | AS | mA*fU*fUmGfCmUmUmUmUmAmUfUmCfCmAfAmGmAmAmC | 676 |
| | | mU*mC*mU | |
| Modified | SS | mA*mG*mUmUmCmUmUmGfGfAfAmUmAmAmAmAmGmCmAmA | 667 |
| duplex 11 | | mU | |
| | AS | mA*fU*mUmGdCmUdTmUmUmAmUfUmCfCmAfAmGmAmAmC | 677 |
| | | mU*mC*mU | |
| Modified | SS | mA*mG*mUmUmCmUmUmGfGfAfAmUmAmAmAmAmGmCmAmA | 667 |
| | | mU | |
| duplex 12 | AS | mA*fU*mUmGdCmUmUmUmUmAmUfUmCfCmAfAmGmAmAmCm | 678 |
| | | U*mC*mU | |
| Modified | SS | mA*mG*mUmUmCmUmUmGfGfAfAmUmAmAmAmAmGmCmAmA | 667 |
| | | mU | |
| duplex 13 | AS | mA*fU*mUmGmCmUgnaTmUmUmAmUfUmCfCmAfAmGmAmAmC | 679 |
| | | mU*mC*mU | |
| Modified | SS | mA*mG*mUmUmCmUfUmGfGfAfAmUmAmAmAmAmGmCmAmAm | 672 |
| | | U | |
| duplex 14 | AS | mA*fU*mUmGfCmUmUmUmUmAmUfUmCfCmAfAmGmAmAmCm | 669 |
| | | U*mC*mU | |
| Modified | SS | mA*mG*mUmUmCmUfUmGfGfAfAmUfAmAmAmAmGmCmAmAm | 680 |
| | | U | |
| duplex 15 | AS | mA*fU*mUmGdCmUmUmUmUmAmUfUmCfCmAfAmGmAmAmCm | 678 |
| | | U*mC*mU | |
| Modified | SS | mA*mG*mUmUmCmUfUmGfGfAfAmUfAmAfAmAmGmCmAmAmU | 681 |
| duplex 16 | AS | mA*fU*mUmGdCmUmUmUmUmAmUfUmCfCmAfAmGmAmAmCm | 678 |
| | | U*mC*mU | |
| Modified | SS | mA*mG*mUmUmCmUmUfGfGfAfAmUmAmAmAmAmGmCmAmAm | 682 |
| | | U | |
| duplex 17 | AS | mA*fU*mUmGdCmUmUmUmUmAmUfUmCfCmAfAmGmAmAmCm | 678 |
| | | U*mC*mU | |
| Modified | SS | mA*mG*mUmUmCmUfUmGfGfAdAmUmAmAmAmAmGmCmAmAm | 683 |
| | | U | |
| duplex 18 | AS | mA*fU*mUmGfCmUmUmUmUmAmUfUmCfCmAfAmGmAmAmCm | 669 |
| | | U*mC*mU | |
| Modified | SS | mA*mG*mUmUmCmUfUmGfGfAdAmUmAmAmAmAmGmCmAmAm | 683 |
| | | U | |
| duplex 19 | AS | mA*fU*mUmGdCmUmUmUmUmAmUfUmCfCmAfAmGmAmAmCm | 678 |
| | | U*mC*mU | |
| Modified | SS | mA*mG*mUmUmCmUfUmGfGfAdAmUmAmAmAmAmGmCmAmAm | 683 |
| | | U | |
| duplex 20 | AS | mA*fU*mUmGdCmUdTmUmUmAmUmUmCfCmAfAmGmAmAmCm | 684 |
| | | U*mC*mU | |
| Modified | SS | mA*mG*mUmUfCmUfUmGfGfAfAmUfAmAmAmAmGmCmAmAmU | 685 |
| duplex 21 | AS | mA*fU*mUmGdCmUmUmUmUmAmUfUmCfCmAfAmGmAmAmCm | 678 |
| | | U*mC*mU | |

Specifically, uppercase C, G, U, and A represent the base composition of a nucleotide; lowercase d represents that the nucleotide immediately to the right of the letter d is a deoxyribonucleotide; lowercase m represents that the nucleotide immediately to the right of the letter m is a 2′-methoxy nucleotide; symbol f represents that the nucleotide immediately to the right of the symbol f is a 2-fluoro nucleotide; gn represents that the nucleotide immediately to the right of the symbol gn is a glycerol nucleotide (GNA); tn represents that the nucleotide immediately to the right of the symbol tn is a threose nucleotide (TNA); ml refers to inosine with a 2′-methoxy substitution on the ribose moiety; symbol * represents that the two adjacent nucleotides flanking the symbol * are linked via a phosphorothioate linkage.

### Example 2: In Vitro Activity Assay of ANGPTL4 siRNA—psiCHECK

Potential ANGPTL4 inhibitors of the present disclosure were synthesized by bioinformatics analysis to screen cross-reactive ANGPTL4 siRNA candidate sequences targeting both human and non-human primates. To screen for the target siRNA, the human ANGPTL4 cDNA sequence (NCBI Accession No. NM 139314.3) was cloned from a commercial available mammalian expression vector (OriGene, Rockville, MD) into a commercially available reporter-based screening plasmid, psiCHECK 2 (Promega, Madison, WI), which generated a Renilla luciferase/ANGPTL4 fusion mRNA. psiCHECK screening was performed in 293T cells (Nanjing Cobioer Biosciences) to assess siRNA activity. 293T cells were inoculated into a 96-well plate at 20,000 cells/well. The cells were co-transfected with the ANGPTL4 siRNA of the present disclosure at two concentrations, 50 ng ofANGPTL4-psiCHECK 2 plasmid per well and 0.3 μL of Lipofectamine 2000™ per well, respectively. After 24 hours of incubation at 37° C. with 5% CO₂, the Dual-Glo Luciferase Assay System (Promega, E2920) was used to perform a dual luciferase reporter assay for ANGPTL4 knockdown activity. 3 to 4 independent transfections were performed per duplex. Gene knockdown was determined by measuring Renilla luciferase levels normalized to constitutively expressed firefly luciferase levels (Tables 4 to 9). In Tables 4 to 9, “Average” represents the mean of the ratio of the ANGPTL4 expression in the detected sample to that in the control (without siRNA), and “SD” represents the standard deviation. PC a (an unmodified sequence of the EDT01162 molecule from published patent WO_2022261005), known to have an inhibitory effect on the ANGPTL4 gene, was used as a positive control in the activity assay of duplexes.

TABLE 4
Results of in vitro activity assay of duplexes

| | Relative Rluc-ANGPTL4 expression | | | | | Relative Rluc-ANGPTL4 expression | | | |
|---|---|---|---|---|---|---|---|---|---|
| Duplex | 1 nM | | 0.1 nM | | Duplex | 1 nM | | 0.1 nM | |
| No. | Average | SD | Average | SD | No. | Average | SD | Average | SD |
| 1 | 0.83 | 0.06 | 0.97 | 0.03 | 105 | 0.64 | 0.06 | 0.85 | 0.08 |
| 2 | 0.71 | 0.06 | 0.88 | 0.05 | 106 | 0.61 | 0.06 | 0.70 | 0.12 |
| 3 | 1.05 | 0.10 | 1.02 | 0.05 | 107 | 0.48 | 0.09 | 0.66 | 0.07 |
| 4 | 0.54 | 0.03 | 0.64 | 0.04 | 108 | 0.58 | 0.06 | 0.92 | 0.06 |
| 5 | 0.54 | 0.05 | 0.67 | 0.07 | 109 | 0.77 | 0.17 | 0.96 | 0.13 |
| 6 | 0.52 | 0.05 | 0.77 | 0.04 | 110 | 0.79 | 0.09 | 0.98 | 0.17 |
| 7 | 0.46 | 0.04 | 0.60 | 0.04 | 111 | 0.84 | 0.19 | 0.97 | 0.01 |
| 8 | 0.63 | 0.06 | 0.88 | 0.07 | 112 | 0.80 | 0.13 | 0.90 | 0.09 |
| 9 | 0.66 | 0.06 | 0.73 | 0.02 | 113 | 0.87 | 0.23 | 1.15 | 0.09 |
| 10 | 0.53 | 0.06 | 0.80 | 0.09 | 114 | 0.83 | 0.05 | 0.98 | 0.13 |
| 11 | 0.79 | 0.15 | 1.04 | 0.10 | 115 | 0.81 | 0.05 | 0.98 | 0.07 |
| 12 | 0.73 | 0.04 | 0.79 | 0.07 | 116 | 0.70 | 0.11 | 0.89 | 0.07 |
| 13 | 0.51 | 0.01 | 0.73 | 0.03 | 117 | 1.05 | 0.04 | 0.91 | 0.08 |
| 14 | 0.60 | 0.02 | 0.73 | 0.04 | 118 | 0.97 | 0.13 | 1.03 | 0.17 |
| 15 | 0.60 | 0.04 | 0.86 | 0.06 | 119 | 0.66 | 0.15 | 0.90 | 0.04 |
| 16 | 0.55 | 0.01 | 0.82 | 0.13 | 120 | 0.53 | 0.03 | 0.81 | 0.04 |
| 17 | 1.05 | 0.03 | 1.00 | 0.21 | 121 | 0.63 | 0.03 | 0.79 | 0.04 |
| 18 | 0.71 | 0.05 | 0.90 | 0.07 | 122 | 0.58 | 0.10 | 0.82 | 0.11 |
| 19 | 0.82 | 0.04 | 1.01 | 0.04 | 123 | 0.62 | 0.08 | 0.74 | 0.10 |
| 20 | 0.61 | 0.06 | 0.79 | 0.05 | 124 | 0.81 | 0.08 | 0.92 | 0.07 |
| 21 | 0.85 | 0.11 | 1.00 | 0.16 | 125 | 0.55 | 0.19 | 0.57 | 0.09 |
| 22 | 0.76 | 0.05 | 0.88 | 0.05 | 126 | 0.48 | 0.05 | 0.76 | 0.04 |
| 23 | 0.35 | 0.09 | 0.59 | 0.06 | 127 | 0.67 | 0.06 | 0.81 | 0.10 |
| 24 | 0.59 | 0.04 | 0.79 | 0.06 | 128 | 0.73 | 0.07 | 0.86 | 0.09 |
| 25 | 0.57 | 0.09 | 0.81 | 0.06 | 129 | 0.87 | 0.09 | 1.09 | 0.12 |
| 26 | 0.60 | 0.02 | 0.79 | 0.07 | 130 | 0.62 | 0.08 | 0.84 | 0.11 |
| 27 | 0.84 | 0.05 | 0.79 | 0.02 | 131 | 0.36 | 0.14 | 0.56 | 0.08 |
| 28 | 0.69 | 0.05 | 0.86 | 0.09 | 132 | 0.42 | 0.09 | 0.75 | 0.05 |
| 29 | 0.36 | 0.07 | 0.56 | 0.11 | 133 | 0.35 | 0.09 | 0.73 | 0.16 |
| 30 | 0.78 | 0.05 | 0.93 | 0.08 | 134 | 0.67 | 0.10 | 0.79 | 0.06 |
| 31 | 0.75 | 0.05 | 0.88 | 0.05 | 135 | 0.65 | 0.03 | 1.05 | 0.15 |
| 32 | 0.68 | 0.02 | 0.87 | 0.12 | 136 | 0.74 | 0.14 | 0.90 | 0.06 |
| 33 | 0.33 | 0.06 | 0.47 | 0.07 | 137 | 0.76 | 0.05 | 0.93 | 0.07 |
| 34 | 0.38 | 0.01 | 0.61 | 0.04 | 138 | 0.85 | 0.14 | 0.84 | 0.11 |
| 35 | 0.50 | 0.07 | 0.66 | 0.10 | 139 | 0.62 | 0.10 | 0.86 | 0.04 |
| 36 | 0.74 | 0.09 | 1.00 | 0.06 | 140 | 0.38 | 0.15 | 0.57 | 0.18 |
| 37 | 0.37 | 0.03 | 0.52 | 0.08 | 141 | 0.54 | 0.04 | 0.57 | 0.06 |
| 38 | 0.59 | 0.05 | 0.85 | 0.07 | 142 | 0.55 | 0.09 | 0.79 | 0.03 |
| 39 | 0.38 | 0.10 | 0.71 | 0.14 | 143 | 0.25 | 0.06 | 0.33 | 0.01 |
| 40 | 1.08 | 0.02 | 0.99 | 0.02 | 144 | 0.21 | 0.05 | 0.35 | 0.05 |
| 41 | 0.61 | 0.06 | 0.75 | 0.06 | 145 | 0.38 | 0.09 | 0.63 | 0.09 |
| 42 | 0.60 | 0.04 | 0.79 | 0.05 | 146 | 0.29 | 0.04 | 0.37 | 0.07 |
| 43 | 0.45 | 0.03 | 0.63 | 0.02 | 147 | 0.27 | 0.03 | 0.44 | 0.08 |
| 44 | 0.95 | 0.11 | 1.03 | 0.05 | 148 | 0.19 | 0.08 | 0.32 | 0.10 |
| 45 | 0.57 | 0.03 | 0.73 | 0.04 | 149 | 0.22 | 0.08 | 0.25 | 0.05 |
| 46 | 0.58 | 0.06 | 0.79 | 0.12 | 150 | 0.64 | 0.06 | 0.69 | 0.05 |
| 47 | 0.45 | 0.03 | 0.61 | 0.03 | 151 | 0.44 | 0.03 | 0.64 | 0.20 |
| 48 | 0.66 | 0.10 | 0.66 | 0.03 | 152 | 0.65 | 0.15 | 0.75 | 0.11 |
| 49 | 0.54 | 0.02 | 0.94 | 0.14 | 153 | 0.41 | 0.02 | 0.69 | 0.11 |
| 50 | 0.68 | 0.07 | 0.80 | 0.03 | 154 | 0.55 | 0.13 | 0.66 | 0.10 |
| 51 | 1.13 | 0.11 | 1.15 | 0.20 | 155 | 0.54 | 0.08 | 0.75 | 0.07 |
| 52 | 1.02 | 0.24 | 1.15 | 0.18 | 156 | 0.76 | 0.09 | 0.89 | 0.09 |
| 53 | 0.40 | 0.03 | 0.59 | 0.03 | 157 | 0.49 | 0.08 | 0.64 | 0.07 |
| 54 | 0.89 | 0.06 | 0.99 | 0.05 | 158 | 0.61 | 0.18 | 0.83 | 0.16 |
| 55 | 0.87 | 0.07 | 0.96 | 0.08 | 159 | 0.86 | 0.11 | 1.11 | 0.04 |
| 56 | 0.94 | 0.11 | 0.99 | 0.10 | 160 | 0.43 | 0.08 | 0.56 | 0.13 |
| 57 | 0.42 | 0.07 | 0.59 | 0.06 | 161 | 0.63 | 0.06 | 0.81 | 0.11 |
| 58 | 0.64 | 0.06 | 0.88 | 0.11 | 162 | 0.53 | 0.02 | 0.84 | 0.12 |
| 59 | 0.42 | 0.06 | 0.61 | 0.09 | 163 | 0.61 | 0.08 | 0.73 | 0.09 |
| 60 | 0.45 | 0.05 | 0.56 | 0.04 | 164 | 0.50 | 0.11 | 0.68 | 0.11 |
| 61 | 0.82 | 0.06 | 1.09 | 0.10 | 165 | 0.54 | 0.05 | 0.63 | 0.08 |
| 62 | 0.64 | 0.04 | 0.82 | 0.06 | 166 | 0.53 | 0.06 | 0.65 | 0.06 |
| 63 | 0.72 | 0.04 | 0.83 | 0.03 | 167 | 0.49 | 0.03 | 0.64 | 0.03 |
| 64 | 0.63 | 0.04 | 0.80 | 0.07 | 168 | 0.53 | 0.07 | 0.70 | 0.07 |
| 65 | 0.41 | 0.05 | 0.71 | 0.11 | 169 | 0.48 | 0.02 | 0.78 | 0.02 |
| 66 | 0.83 | 0.03 | 0.99 | 0.03 | 170 | 0.93 | 0.04 | 0.94 | 0.10 |
| 67 | 0.36 | 0.06 | 0.70 | 0.15 | 171 | 0.72 | 0.04 | 0.86 | 0.11 |
| 68 | 1.00 | 0.06 | 1.09 | 0.19 | 172 | 0.73 | 0.06 | 0.75 | 0.07 |
| 69 | 0.79 | 0.07 | 0.99 | 0.06 | 173 | 0.87 | 0.14 | 0.83 | 0.05 |
| 70 | 0.89 | 0.02 | 1.08 | 0.02 | 174 | 0.94 | 0.07 | 1.16 | 0.14 |
| 71 | 0.64 | 0.04 | 0.82 | 0.03 | 175 | 0.72 | 0.10 | 0.86 | 0.03 |
| 72 | 0.55 | 0.09 | 0.72 | 0.10 | 176 | 0.67 | 0.09 | 0.86 | 0.05 |
| 73 | 0.59 | 0.04 | 0.81 | 0.03 | 177 | 0.38 | 0.03 | 0.58 | 0.09 |
| 74 | 0.36 | 0.08 | 0.57 | 0.08 | 178 | 0.87 | 0.07 | 0.98 | 0.15 |
| 75 | 0.63 | 0.04 | 0.78 | 0.04 | 179 | 0.49 | 0.05 | 0.54 | 0.06 |
| 76 | 0.60 | 0.08 | 0.87 | 0.08 | 180 | 0.57 | 0.06 | 0.65 | 0.06 |
| 77 | 0.55 | 0.02 | 0.76 | 0.04 | 181 | 0.48 | 0.06 | 0.62 | 0.05 |
| 78 | 0.39 | 0.05 | 0.51 | 0.02 | 182 | 0.64 | 0.11 | 0.65 | 0.08 |
| 79 | 0.54 | 0.03 | 0.79 | 0.04 | 183 | 0.56 | 0.03 | 0.65 | 0.02 |
| 80 | 0.49 | 0.06 | 0.59 | 0.08 | 184 | 0.57 | 0.08 | 0.71 | 0.09 |
| 81 | 0.46 | 0.03 | 0.72 | 0.02 | 185 | 0.47 | 0.03 | 0.65 | 0.08 |
| 82 | 0.76 | 0.06 | 0.81 | 0.11 | 186 | 0.44 | 0.06 | 0.60 | 0.07 |
| 83 | 0.52 | 0.02 | 0.80 | 0.04 | 187 | 0.39 | 0.07 | 0.43 | 0.04 |
| 84 | 0.50 | 0.04 | 0.69 | 0.00 | 188 | 0.37 | 0.02 | 0.45 | 0.05 |
| 85 | 0.59 | 0.22 | 1.05 | 0.08 | 189 | 0.64 | 0.31 | 0.80 | 0.05 |
| 86 | 0.60 | 0.03 | 0.76 | 0.06 | 190 | 0.54 | 0.30 | 0.52 | 0.07 |
| 87 | 0.54 | 0.10 | 0.76 | 0.21 | 191 | 0.74 | 0.07 | 0.84 | 0.07 |
| 88 | 0.68 | 0.07 | 0.79 | 0.16 | 192 | 0.92 | 0.04 | 0.90 | 0.08 |
| 89 | 0.76 | 0.10 | 0.93 | 0.12 | 193 | 0.92 | 0.06 | 1.01 | 0.06 |
| 90 | 0.66 | 0.21 | 0.73 | 0.05 | 194 | 0.49 | 0.03 | 0.82 | 0.13 |
| 91 | 0.74 | 0.10 | 0.91 | 0.08 | 195 | 0.50 | 0.03 | 0.91 | 0.11 |
| 92 | 0.69 | 0.09 | 0.77 | 0.15 | 196 | 0.43 | 0.05 | 0.71 | 0.11 |
| 93 | 0.89 | 0.08 | 0.89 | 0.10 | 197 | 0.70 | 0.08 | 0.93 | 0.05 |
| 94 | 0.50 | 0.06 | 0.86 | 0.04 | 198 | 0.63 | 0.03 | 0.89 | 0.05 |
| 95 | 0.57 | 0.15 | 0.89 | 0.23 | 199 | 0.69 | 0.05 | 0.81 | 0.03 |
| 96 | 0.51 | 0.14 | 0.88 | 0.21 | 200 | 0.84 | 0.04 | 0.84 | 0.07 |
| 97 | 0.63 | 0.08 | 0.71 | 0.09 | 201 | 0.67 | 0.05 | 0.93 | 0.07 |
| 98 | 0.90 | 0.15 | 1.02 | 0.17 | 202 | 0.68 | 0.04 | 0.84 | 0.10 |
| 99 | 1.03 | 0.30 | 1.04 | 0.18 | 203 | 0.59 | 0.11 | 0.81 | 0.03 |
| 100 | 0.87 | 0.13 | 1.25 | 0.21 | 204 | 0.65 | 0.09 | 0.87 | 0.10 |
| 101 | 0.83 | 0.05 | 0.94 | 0.08 | 205 | 0.48 | 0.03 | 0.68 | 0.11 |
| 102 | 0.63 | 0.08 | 0.94 | 0.20 | 206 | 0.40 | 0.09 | 0.52 | 0.06 |
| 103 | 0.55 | 0.11 | 0.73 | 0.14 | 207 | 0.44 | 0.02 | 0.49 | 0.03 |
| 104 | 0.54 | 0.05 | 0.72 | 0.12 | 208 | 0.34 | 0.05 | 0.38 | 0.06 |
| PC a | 0.31 | 0.03 | 0.36 | 0.12 | | | | | |

TABLE 5
Results of in vitro activity assay of duplexes

| Relative Rluc-ANGPTL4 expression | |
|---|---|
| 0.5 nM | 0.05 nM |

| Duplex No. | Average | SD | Average | SD |
|---|---|---|---|---|
| 143 | 0.215 | 0.023 | 0.309 | 0.028 |
| 212 | 0.337 | 0.031 | 0.547 | 0.034 |
| 213 | 0.177 | 0.018 | 0.300 | 0.047 |
| 214 | 0.199 | 0.034 | 0.380 | 0.037 |
| 215 | 0.326 | 0.039 | 0.603 | 0.045 |
| 217 | 0.277 | 0.044 | 0.460 | 0.030 |

TABLE 6
Results of in vitro activity assay of conjugates

| | Relative Rluc-ANGPTL4 expression | | | | | Relative Rluc-ANGPTL4 expression | | | |
|---|---|---|---|---|---|---|---|---|---|
| Conjugate | 1 nM | | 0.1 nM | | Conjugate | 1 nM | | 0.1 nM | |
| No. | Average | SD | Average | SD | No. | Average | SD | Average | SD |
| 1 | 0.18 | 0.03 | 0.19 | 0.03 | 25 | 0.17 | 0.05 | 0.33 | 0.04 |
| 2 | 0.17 | 0.03 | 0.50 | 0.04 | 26 | 0.18 | 0.04 | 0.38 | 0.04 |
| 3 | 0.17 | 0.06 | 0.44 | 0.12 | 30 | 0.17 | 0.03 | 0.22 | 0.05 |
| 4 | 0.16 | 0.04 | 0.51 | 0.05 | 31 | 0.15 | 0.02 | 0.33 | 0.04 |
| 5 | 0.16 | 0.03 | 0.22 | 0.05 | 29 | 0.22 | 0.06 | 0.29 | 0.04 |
| 6 | 0.26 | 0.05 | 0.65 | 0.15 | 32 | 0.15 | 0.03 | 0.22 | 0.05 |
| 7 | 0.25 | 0.03 | 0.46 | 0.07 | 33 | 0.17 | 0.01 | 0.21 | 0.03 |
| 8 | 0.25 | 0.05 | 0.50 | 0.09 | 34 | 0.19 | 0.03 | 0.29 | 0.05 |
| 9 | 0.24 | 0.06 | 0.41 | 0.09 | 35 | 0.17 | 0.03 | 0.20 | 0.03 |
| 11 | 0.25 | 0.07 | 0.50 | 0.13 | 36 | 0.21 | 0.07 | 0.31 | 0.06 |
| 10 | 0.27 | 0.07 | 0.51 | 0.04 | 42 | 0.21 | 0.05 | 0.48 | 0.10 |
| 12 | 0.36 | 0.04 | 0.73 | 0.05 | 43 | 0.24 | 0.04 | 0.54 | 0.03 |
| 13 | 0.18 | 0.07 | 0.24 | 0.09 | 44 | 0.21 | 0.04 | 0.43 | 0.04 |
| 19 | 0.15 | 0.03 | 0.17 | 0.07 | 45 | 0.22 | 0.04 | 0.52 | 0.01 |
| 17 | 0.14 | 0.04 | 0.23 | 0.06 | 46 | 0.24 | 0.03 | 0.52 | 0.07 |
| 23 | 0.12 | 0.03 | 0.22 | 0.04 | 37 | 0.29 | 0.03 | 0.44 | 0.05 |
| 14 | 0.23 | 0.03 | 0.32 | 0.07 | 38 | 0.28 | 0.05 | 0.50 | 0.03 |
| 20 | 0.18 | 0.02 | 0.26 | 0.03 | 39 | 0.30 | 0.05 | 0.56 | 0.05 |
| 15 | 0.13 | 0.05 | 0.25 | 0.05 | 40 | 0.26 | 0.08 | 0.53 | 0.02 |
| 21 | 0.14 | 0.04 | 0.19 | 0.03 | 41 | 0.31 | 0.04 | 0.61 | 0.12 |
| 18 | 0.19 | 0.03 | 0.26 | 0.05 | 47 | 0.20 | 0.03 | 0.33 | 0.07 |
| 24 | 0.19 | 0.06 | 0.16 | 0.06 | 48 | 0.21 | 0.05 | 0.39 | 0.10 |
| 16 | 0.15 | 0.04 | 0.26 | 0.05 | 49 | 0.24 | 0.02 | 0.54 | 0.08 |
| 22 | 0.16 | 0.04 | 0.23 | 0.02 | 50 | 0.18 | 0.03 | 0.30 | 0.05 |
| 27 | 0.19 | 0.07 | 0.19 | 0.06 | 51 | 0.19 | 0.05 | 0.41 | 0.04 |
| 28 | 0.16 | 0.04 | 0.24 | 0.07 | 52 | 0.26 | 0.05 | 0.54 | 0.08 |

TABLE 7
Results of in vitro activity assay of conjugates

| | Relative Rluc-ANGPTL4 expression | | | | | Relative Rluc-ANGPTL4 expression | | | |
|---|---|---|---|---|---|---|---|---|---|
| Conjugate | 1 nM | | 0.1 nM | | Conjugate | 1 nM | | 0.1 nM | |
| No. | Average | SD | Average | SD | No. | Average | SD | Average | SD |
| 5 | 0.17 | 0.05 | 0.22 | 0.07 | 74 | 0.17 | 0.02 | 0.28 | 0.03 |
| 54 | 0.20 | 0.04 | 0.31 | 0.04 | 77 | 0.12 | 0.03 | 0.19 | 0.03 |
| 60 | 0.17 | 0.04 | 0.22 | 0.03 | 83 | 0.12 | 0.04 | 0.23 | 0.02 |
| 57 | 0.18 | 0.03 | 0.26 | 0.06 | 80 | 0.12 | 0.03 | 0.20 | 0.03 |
| 63 | 0.17 | 0.05 | 0.20 | 0.04 | 32 | 0.17 | 0.02 | 0.28 | 0.04 |
| 69 | 0.17 | 0.03 | 0.20 | 0.05 | 55 | 0.18 | 0.04 | 0.27 | 0.04 |
| 94 | 0.12 | 0.07 | 0.20 | 0.04 | 61 | 0.19 | 0.04 | 0.29 | 0.06 |
| 91 | 0.17 | 0.03 | 0.33 | 0.05 | 58 | 0.17 | 0.06 | 0.20 | 0.03 |
| 87 | 0.18 | 0.05 | 0.24 | 0.04 | 64 | 0.15 | 0.05 | 0.21 | 0.05 |
| 75 | 0.29 | 0.05 | 0.35 | 0.05 | 70 | 0.14 | 0.01 | 0.29 | 0.04 |
| 78 | 0.19 | 0.05 | 0.24 | 0.06 | 92 | 0.13 | 0.02 | 0.16 | 0.01 |
| 81 | 0.15 | 0.02 | 0.23 | 0.01 | 25 | 0.16 | 0.03 | 0.23 | 0.02 |
| 84 | 0.22 | 0.05 | 0.30 | 0.07 | 73 | 0.15 | 0.02 | 0.30 | 0.03 |
| 93 | 0.15 | 0.04 | 0.13 | 0.02 | 88 | 0.13 | 0.04 | 0.25 | 0.04 |
| 23 | 0.11 | 0.04 | 0.16 | 0.05 | 76 | 0.13 | 0.02 | 0.32 | 0.10 |
| 19 | 0.14 | 0.03 | 0.16 | 0.05 | 85 | 0.17 | 0.03 | 0.23 | 0.04 |
| 53 | 0.16 | 0.02 | 0.23 | 0.04 | 82 | 0.14 | 0.04 | 0.25 | 0.03 |
| 59 | 0.21 | 0.02 | 0.21 | 0.05 | 79 | 0.15 | 0.00 | 0.26 | 0.08 |
| 56 | 0.16 | 0.04 | 0.26 | 0.04 | 27 | 0.12 | 0.05 | 0.22 | 0.04 |
| 68 | 0.13 | 0.03 | 0.24 | 0.05 | 89 | 0.64 | 0.03 | 0.89 | 0.04 |
| 1 | 0.13 | 0.02 | 0.20 | 0.07 | 42 | 0.24 | 0.02 | 0.43 | 0.06 |
| 90 | 0.15 | 0.02 | 0.21 | 0.04 | 95 | 0.50 | 0.03 | 0.80 | 0.11 |
| 86 | 0.12 | 0.03 | 0.26 | 0.02 | | | | | |

TABLE 8
Results of in vitro activity assay of conjugates

| | Relative Rluc-ANGPTL4 expression | | | | | Relative Rluc-ANGPTL4 expression | | | |
|---|---|---|---|---|---|---|---|---|---|
| Conjugate | 0.5 nM | | 0.05 nM | | Conjugate | 0.5 nM | | 0.05 nM | |
| No. | Average | SD | Average | SD | No. | Average | SD | Average | SD |
| 1 | 0.15 | 0.03 | 0.34 | 0.05 | 134 | 0.15 | 0.03 | 0.25 | 0.01 |
| 90 | 0.14 | 0.02 | 0.39 | 0.08 | 123 | 0.22 | 0.02 | 0.29 | 0.04 |
| 71 | 0.19 | 0.02 | 0.43 | 0.03 | 124 | 0.24 | 0.03 | 0.32 | 0.07 |
| 62 | 0.18 | 0.02 | 0.35 | 0.02 | 125 | 0.39 | 0.04 | 0.41 | 0.06 |
| 65 | 0.15 | 0.02 | 0.30 | 0.04 | 126 | 0.26 | 0.01 | 0.34 | 0.05 |
| 101 | 0.15 | 0.03 | 0.44 | 0.04 | 127 | 0.22 | 0.02 | 0.28 | 0.02 |
| 102 | 0.17 | 0.03 | 0.41 | 0.05 | 128 | 0.17 | 0.02 | 0.29 | 0.04 |
| 103 | 0.19 | 0.02 | 0.39 | 0.05 | 129 | 0.24 | 0.05 | 0.40 | 0.02 |
| 104 | 0.15 | 0.02 | 0.43 | 0.05 | 93 | 0.19 | 0.04 | 0.42 | 0.06 |
| 93 | 0.14 | 0.02 | 0.28 | 0.05 | 229 | 0.13 | 0.03 | 0.19 | 0.04 |
| 130 | 0.13 | 0.03 | 0.29 | 0.02 | 230 | 0.11 | 0.05 | 0.23 | 0.07 |
| 23 | 0.14 | 0.02 | 0.25 | 0.04 | 231 | 0.15 | 0.05 | 0.26 | 0.06 |
| 132 | 0.13 | 0.02 | 0.26 | 0.01 | 77 | 0.17 | 0.02 | 0.36 | 0.04 |
| 19 | 0.16 | 0.01 | 0.28 | 0.03 | 232 | 0.11 | 0.02 | 0.26 | 0.07 |
| 133 | 0.13 | 0.02 | 0.28 | 0.03 | 80 | 0.11 | 0.05 | 0.29 | 0.04 |
| 5 | 0.17 | 0.02 | 0.30 | 0.03 | 233 | 0.12 | 0.03 | 0.28 | 0.04 |
| 72 | 0.20 | 0.03 | 0.52 | 0.03 | 234 | 0.10 | 0.01 | 0.27 | 0.07 |
| 66 | 0.18 | 0.03 | 0.41 | 0.03 | 235 | 0.12 | 0.02 | 0.23 | 0.01 |
| 94 | 0.15 | 0.01 | 0.28 | 0.02 | 32 | 0.13 | 0.04 | 0.34 | 0.05 |
| 131 | 0.15 | 0.02 | 0.28 | 0.04 | 67 | 0.12 | 0.01 | 0.39 | 0.07 |
| 32 | 0.18 | 0.02 | 0.37 | 0.04 | 92 | 0.11 | 0.03 | 0.19 | 0.02 |
| 67 | 0.18 | 0.02 | 0.39 | 0.01 | 64 | 0.13 | 0.04 | 0.34 | 0.04 |
| 112 | 0.22 | 0.02 | 0.64 | 0.10 | 236 | 0.16 | 0.03 | 0.43 | 0.02 |
| 113 | 0.23 | 0.04 | 0.67 | 0.05 | 92 | 0.13 | 0.01 | 0.21 | 0.04 |
| 114 | 0.21 | 0.02 | 0.65 | 0.03 | 237 | 0.12 | 0.04 | 0.19 | 0.05 |
| 50 | 0.20 | 0.03 | 0.49 | 0.05 | 238 | 0.10 | 0.03 | 0.22 | 0.04 |
| 134 | 0.15 | 0.03 | 0.25 | 0.01 | 239 | 0.15 | 0.05 | 0.22 | 0.02 |
| 107 | 0.15 | 0.01 | 0.45 | 0.03 | 26 | 0.18 | 0.05 | 0.56 | 0.06 |
| 106 | 0.16 | 0.02 | 0.36 | 0.05 | 240 | 0.15 | 0.01 | 0.23 | 0.03 |
| 105 | 0.16 | 0.04 | 0.39 | 0.03 | 241 | 0.10 | 0.04 | 0.22 | 0.05 |
| 108 | 0.16 | 0.02 | 0.35 | 0.08 | 242 | 0.14 | 0.02 | 0.30 | 0.02 |
| 109 | 0.16 | 0.02 | 0.17 | 0.04 | 225 | 0.13 | 0.02 | 0.27 | 0.03 |
| 110 | 0.17 | 0.02 | 0.44 | 0.04 | 127 | 0.22 | 0.02 | 0.28 | 0.02 |
| 111 | 0.12 | 0.04 | 0.25 | 0.02 | 128 | 0.17 | 0.02 | 0.29 | 0.04 |
| 50 | 0.23 | 0.03 | 0.55 | 0.07 | 129 | 0.24 | 0.05 | 0.40 | 0.02 |
| 123 | 0.22 | 0.02 | 0.29 | 0.04 | 109 | 0.16 | 0.02 | 0.17 | 0.04 |
| 124 | 0.24 | 0.03 | 0.32 | 0.07 | 111 | 0.12 | 0.04 | 0.25 | 0.02 |
| 125 | 0.39 | 0.04 | 0.41 | 0.06 | 119 | 0.15 | 0.03 | 0.33 | 0.03 |
| 126 | 0.26 | 0.01 | 0.34 | 0.05 | 120 | 0.18 | 0.05 | 0.19 | 0.07 |

TABLE 9
Results of in vitro activity assay of modified duplexes

| | Relative Rluc-ANGPTL4 expression | | | |
|---|---|---|---|---|
| | 0.5 nM | | 0.05 nM | |
| Modified duplex No. | Average | SD | Average | SD |
| 1 | 0.132 | 0.041 | 0.233 | 0.044 |
| 2 | 0.129 | 0.019 | 0.203 | 0.020 |
| 3 | 0.181 | 0.027 | 0.301 | 0.030 |
| 4 | 0.137 | 0.004 | 0.223 | 0.038 |
| 5 | 0.157 | 0.029 | 0.277 | 0.025 |
| 6 | 0.167 | 0.010 | 0.240 | 0.027 |
| 7 | 0.164 | 0.014 | 0.317 | 0.032 |
| 8 | 0.205 | 0.021 | 0.258 | 0.041 |
| 9 | 0.162 | 0.021 | 0.213 | 0.029 |
| 10 | 0.142 | 0.024 | 0.179 | 0.039 |
| 11 | 0.151 | 0.020 | 0.256 | 0.024 |
| 12 | 0.149 | 0.037 | 0.241 | 0.014 |
| 13 | 0.198 | 0.025 | 0.352 | 0.021 |
| 14 | 0.142 | 0.029 | 0.217 | 0.020 |
| 15 | 0.158 | 0.010 | 0.266 | 0.044 |
| 16 | 0.144 | 0.009 | 0.256 | 0.012 |
| 17 | 0.156 | 0.043 | 0.213 | 0.022 |
| 18 | 0.153 | 0.011 | 0.225 | 0.035 |
| 19 | 0.156 | 0.008 | 0.265 | 0.028 |
| 20 | 0.155 | 0.009 | 0.230 | 0.035 |
| 21 | 0.176 | 0.023 | 0.231 | 0.027 |

The experimental results demonstrated that the siRNA duplexes, siRNA conjugates, and siRNA modified duplexes of the present disclosure exhibited good in vitro inhibitory activity against ANGPTL4 gene expression.

### Example 3: In Vitro Activity Assay of ANGPTL4 RNAi—U138-MG Cell Transfection

U138-MG cells (ATCC) were inoculated into a 24-well plate at 40,000 cells/well and incubated for 16 hours at 37° C. with 5% CO₂ before transfection. The cells were co-transfected with siRNA and Lipofectamine RNAiMAX (Invitrogen). After 24 hours of incubation at 37° C. with 5% CO₂, RNA was extracted using the MolPure Magnetic Tissue/Cell Total RNA Kit (Yeasen, Cat #18600ES60). cDNA synthesis was performed using the gDNA Removal and cDNA Synthesis Kit (TransGen Biotech Co., Ltd., Beijing, China; Cat # AE311-03). Real-time fluorescence PCR was performed using the AACt method on the ABI QuantStudio™ 6 Real-Time PCR System. 3 to 4 independent transfections were performed per duplex or conjugate, with each transfection performed in 3 to 4 replicates (Tables 10 to 11). PC c (EDT01162 from published patent WO_2022261005), known to have an inhibitory effect on the ANGPTL4 gene, as well as PC a, an unmodified sequence of the molecule, were used as positive controls.

TABLE 10
Results of IC₅₀ assay of duplexes

| Duplex No. | IC₅₀ (pM) |
|---|---|
| 143 | 9.68 |
| 144 | 77.58 |
| 146 | 15.90 |
| 147 | 17.92 |
| 148 | 22.98 |
| 149 | 11.97 |
| PC a | 10.14 |

TABLE 11
Results of IC₅₀ assay of conjugates

| Conjugate No. | IC₅₀ (pM) |
|---|---|
| PC c | 31.44 |
| 1 | 6.51 |
| 16 | 12.16 |
| 17 | 17.33 |
| 19 | 1.32 |
| 21 | 7.94 |
| 23 | 3.87 |
| 22 | 13.11 |
| 120 | 7.21 |
| 5 | 33.33 |

The experimental results demonstrated that the siRNA duplexes or siRNA conjugates of the present disclosure exhibited excellent inhibitory activity against ANGPTL4 gene expression in U138-MG cells.

### Example 4: In Vitro Activity Assay of ANGPTL4 RNAi—Free Uptake in Human and Cynomolgus Monkey Primary Hepatocytes

After thawing, human and cynomolgus monkey primary hepatocytes were diluted in culture medium to a density of 600,000 cells/mL. Different concentrations of conjugates were added to a 96-well collagen-coated plate at 10 μL/well, followed by the addition of 90 μL/well of human or cynomolgus monkey primary hepatocytes (54,000 cells/well). A PBS control group was also included. After seeding, the plate was incubated at 37° C. with 5% CO₂ for 48 hours. After 48 hours, the culture medium was removed, and the cells were collected for RNA extraction. Total RNA was extracted using the RNeasy 96 Kit (QIAGEN, Cat #74182) according to the instructions. Subsequently, cDNA was synthesized using HiScript III RT SuperMix for qPCR (+gDNA wiper) (Vazyme, Cat #R323-01) according to the instructions. Real-time fluorescence PCR was performed using the AACt method on the Applied Biosystems QuantStudio 7 Flex Real-Time PCR System (Tables 12 to 13). PC c, known to have an inhibitory effect on the ANGPTL4 gene, was used as a positive control.

TABLE 12
Results of free uptake assay in human primary hepatocytes

| | Free uptake in human primary hepatocytes | | | | | |
|---|---|---|---|---|---|---|
| | 30 nM | | 3 nM | | 0.3 nM | |
| | Average | | Average | | Average | |
| Conjugate | mRNA | | mRNA | | mRNA | |
| No. | remaining | SD | remaining | SD | remaining | SD |
| PC c | 0.11 | 0.02 | 0.22 | 0.02 | 0.58 | 0.05 |
| 1 | 0.13 | 0.01 | 0.26 | 0.01 | 0.58 | 0.05 |
| 17 | 0.13 | 0.04 | 0.25 | 0.01 | 0.64 | 0.06 |
| 23 | 0.12 | 0.02 | 0.25 | 0.01 | 0.61 | 0.02 |
| 19 | 0.11 | 0.02 | 0.25 | 0.01 | 0.58 | 0.04 |
| 28 | 0.17 | 0.02 | 0.35 | 0.02 | 0.68 | 0.03 |
| 16 | 0.12 | 0.01 | 0.26 | 0.02 | 0.62 | 0.06 |
| 22 | 0.13 | 0.01 | 0.32 | 0.01 | 0.69 | 0.02 |
| 15 | 0.14 | 0.02 | 0.33 | 0.01 | 0.80 | 0.05 |
| 21 | 0.13 | 0.02 | 0.27 | 0.01 | 0.71 | 0.02 |
| 93 | NA | NA | 0.29 | 0.07 | 0.81 | 0.07 |
| 227 | NA | NA | 0.26 | 0.04 | 0.76 | 0.02 |
| 133 | NA | NA | 0.32 | 0.07 | 0.89 | 0.01 |
| 92 | NA | NA | 0.26 | 0.02 | 0.53 | 0.08 |
| 234 | NA | NA | 0.31 | 0.01 | 0.61 | 0.13 |
| 235 | NA | NA | 0.35 | 0.02 | 0.68 | 0.14 |
| 236 | NA | NA | 0.30 | 0.04 | 0.61 | 0.12 |
| 238 | NA | NA | 0.28 | 0.03 | 0.65 | 0.06 |
| 225 | NA | NA | 0.74 | 0.07 | 0.93 | 0.14 |
| 120 | NA | NA | 0.37 | 0.03 | 0.73 | 0.13 |

Note:
NA indicates not applicable

TABLE 13
Free uptake assay in cynomolgus monkey primary hepatocytes

| | Free uptake in monkey primary hepatocytes | | | | | |
|---|---|---|---|---|---|---|
| | 30 nM | | 10 nM | | 3 nM | |
| | Average | | Average | | Average | |
| Conjugate | mRNA | | mRNA | | mRNA | |
| No. | remaining | SD | remaining | SD | remaining | SD |
| PC c | 0.35 | 0.08 | 0.39 | 0.16 | 0.53 | 0.08 |
| 5 | 0.39 | 0.13 | 0.41 | 0.07 | 0.43 | 0.08 |
| 1 | 0.23 | 0.01 | 0.41 | 0.16 | 0.54 | 0.15 |
| 17 | 0.34 | 0.15 | 0.45 | 0.04 | 0.57 | 0.06 |
| 23 | 0.36 | 0.07 | 0.53 | 0.08 | 0.65 | 0.32 |
| 19 | 0.35 | 0.03 | 0.40 | 0.04 | 0.78 | 0.24 |
| 16 | 0.29 | 0.09 | 0.43 | 0.09 | 0.56 | 0.07 |

The experimental results demonstrated that the tested conjugates in Table 12 exhibited excellent inhibitory activity against ANGPTL4 gene expression in human primary hepatocytes, while the tested conjugates in Table 13 exhibited excellent inhibitory activity against ANGPTL4 gene expression in cynomolgus monkey primary hepatocytes. Notably, all tested molecules showed a significant inhibitory effect on ANGPTL4 mRNA in both human and monkey primary hepatocytes, with most demonstrating comparable inhibitory activity to the control molecule PC c.

### Example 5: Immunogenicity Assay of ANGPTL4 RNAi

siRNA and control compounds polyIC (MCE, HY-107202) and naked siRNA (i.e., unmodified RNA molecules conventionally used as controls in the art) were transfected into freshly isolated and pooled human PBMCs according to the instructions of Lipofectamine® 3000 Transfection Kit (Thermo, L3000-015), with a final cell density of 20,000 cells/well in a plate. After 24 hours of incubation at 37° C. with 500 CO₂, cell supernatants were collected to measure IFN alpha, IL-6, and TNF alpha levels (Cytokine Kit, Thermo, PPX-03-MXU64WY). The fold change in cytokine levels relative to control wells for each siRNA was calculated to evaluate the induction of different cytokines by siRNA in human PBMCs (Table 14).

The experimental results demonstrated that the controls polyIC, naked siRNA, and GS9688 (CAS: 2004677-13-6) exhibited the expected induction of the three cytokines IFN alpha, IL-6, and TNF alpha at test concentrations.

For IFN alpha, conjugates 130, 133, and 225 exhibited no significant induction (a fold change less than 3) at transfection concentrations of 100 nM and 10 nM, outperforming conjugate PC c. For IL-6, conjugates 130 and 225 exhibited no significant induction at transfection concentrations of 100 nM and 10 nM, outperforming conjugate PC c. For TNF alpha, conjugates 130, 133, 225, and PC c exhibited no significant induction at transfection concentrations of 100 nM and 10 nM.

TABLE 14
Results of in vitro immunogenicity assay of ANGPTL4 RNAi

| | | | IFN alpha | | IL-6 | | TNF alpha | |
|---|---|---|---|---|---|---|---|---|
| | Test | | Average | | Average | | Average | |
| Compound No. | concentration | | fold change | SD | fold change | SD | fold change | SD |
| Conjugate 130 | 100 | nM | 1.62 | 0.36 | 1.91 | 0.87 | 1.20 | 0.28 |
| Conjugate 133 | | | 1.03 | 0.14 | 1.15 | 0.15 | 1.00 | 0.00 |
| Conjugate 225 | | | 1.31 | 0.17 | 0.98 | 0.48 | 1.00 | 0.00 |
| PC c | | | 4.62 | 0.42 | 0.81 | 0.18 | 1.00 | 0.00 |
| polyIC | | | 21.08 | 6.98 | 6.03 | 2.54 | 14.95 | 9.90 |
| Conjugate 130 | 10 | nM | 1.38 | 0.23 | 1.34 | 0.18 | 1.64 | 0.90 |
| Conjugate 133 | | | 1.39 | 0.04 | 5.05 | 3.62 | 1.77 | 1.09 |
| Conjugate 225 | | | 0.94 | 0.06 | 0.86 | 0.12 | 2.20 | 1.70 |
| PC c | | | 2.14 | 0.02 | 3.08 | 0.30 | 1.49 | 0.69 |
| polyIC | | | 26.32 | 0.02 | 2.51 | 0.28 | 2.68 | 0.32 |
| Transfection Regent Control | Control | | 1.00 | 0.05 | 1.00 | 0.20 | 1.00 | 0.00 |
| naked siRNA (9 μg/mL) | | | 30.67 | 7.42 | 2.34 | 0.34 | 6.45 | 1.51 |
| naked siRNA (3 μg/mL) | | | 33.49 | 1.22 | 2.09 | 0.15 | 5.12 | 0.11 |
| GS9688 (8 μM) | | | 8.76 | 0.61 | 739.15 | 73.96 | 476.00 | 24.76 |
| GS9688 (2.5 μM) | | | 4.89 | 1.62 | 762.35 | 57.28 | 523.27 | 28.86 |
| DMSO | | | 1.00 | 0.00 | 1.00 | 0.39 | 1.00 | 0.00 |

### Example 6: In Vivo Activity Assay of ANGPTL4 RNAi

Male hANGPTL4 humanized mice (provided by Jiangsu GemPharmatech) aged 6 to 8 weeks were randomly divided into groups according to body weight, with 6 to 8 mice per group. On Day 1 (D1), the mice were subcutaneously administered PBS, PC c at 3 mpk, PC c at 9 mpk, and conjugate 19 at 3 mpk, respectively. On Day 8 and Day 22 after administration, liver samples were collected from 3 to 4 mice per group, which were fasted overnight before sampling. The hANGPTL4 mRNA levels in the liver were measured by qPCR to compare the knockdown efficiency of different conjugates on the target gene (Table 15). On D8, a single subcutaneous administration of conjugate 19 at 3 mpk showed 92% inhibition of hepatic hANGPTL4 mRNA, outperforming PC c at 3 mpk (78%) and PC c at 9 mpk (90%). On D22, conjugate 19 at 3 mpk still showed 72% inhibition of hepatic hANGPTL4 mRNA, whereas PC c at 3 mpk and PC c at 9 mpk showed only 40% and 42% inhibition, respectively. Conjugate 19 showed better inhibitory activity and persistence on the target gene compared to PC c.

TABLE 15
Inhibition of hepatic hANGPTL4 mRNA
by conjugates on D 8 and D 22

| | |
|---|---|
| D 8 | D 22 |

| Group | Mean | SEM | Mean | SEM |
|---|---|---|---|---|
| PBS | 1.06 | 0.06 | 1.00 | 0.06 |
| PC c, 3 mpk | 0.28 | 0.01 | 0.60 | 0.00 |
| PC c, 9 mpk | 0.10 | 0.00 | 0.58 | 0.01 |
| Conjugate 19, 3 mpk | 0.08 | 0.01 | 0.28 | 0.01 |

In summary, the siRNAs and their conjugates of the present disclosure exhibit good to excellent in vitro inhibitory activity against ANGPTL4 gene expression, can effectively inhibit ANGPTL4 mRNA levels across multiple cell lines, demonstrate satisfactory immunostimulation, and can significantly reduce ANGPTL4 mRNA expression at the animal level.

What is claimed is:

1. An siRNA for inhibiting ANGPTL4 gene expression, wherein the siRNA comprises a sense strand and an antisense strand, wherein the antisense strand comprises at least 17 continuous nucleotides that differ by no more than 4 nucleotides from the nucleotide sequence as shown in SEQ ID NO: 143, and the antisense strand is 17 to 30 nucleotides in length; the sense strand is 17 to 30 nucleotides in length and is at least partially complementary to the antisense strand.

2. The siRNA according to claim 1, wherein the antisense strand is 19 to 27 nucleotides in length, and the sense strand is 19 to 25 nucleotides in length;
preferably, the antisense strand is 19 to 23 nucleotides in length, and the sense strand is 19 to 21 nucleotides in length;
more preferably,
the antisense strand is 23 nucleotides in length, and the sense strand is 21 nucleotides in length; or
the antisense strand is 22 nucleotides in length, and the sense strand is 20 nucleotides in length; or
the antisense strand is 21 nucleotides in length, and the sense strand is 21 nucleotides in length; or
the antisense strand is 21 nucleotides in length, and the sense strand is 19 nucleotides in length; or
the antisense strand is 20 nucleotides in length, and the sense strand is 22 nucleotides in length; or
the antisense strand is 20 nucleotides in length, and the sense strand is 20 nucleotides in length; or
the antisense strand is 27 nucleotides in length, and the sense strand is 25 nucleotides in length; or
the antisense strand is 19 nucleotides in length, and the sense strand is 19 nucleotides in length.

3. The siRNA according to claim 1, wherein the antisense strand differs by no more than 3 nucleotides from the nucleotide sequence as shown in SEQ ID NO: 143;
preferably, the antisense strand differs by no more than 1 nucleotide from the nucleotide sequence as shown in SEQ ID NO: 143;
more preferably, the sequence of the antisense strand is the nucleotide sequence as shown in SEQ ID NO: 143.

4. The siRNA according to claim 1, wherein the sense strand has a mismatch of no more than 3 nucleotides with the antisense strand;
preferably, the sense strand has a mismatch of no more than 1 nucleotide with the antisense strand;
more preferably, the sense strand is fully complementary to the antisense strand;
further more preferably, there is an overhang of 2 nucleotides at the 3′ end of the sense strand; preferably, the 2 nucleotides are reverse complementary to the first two nucleotides at the corresponding position of the starting nucleotide of the sense strand in the transcript as shown in NCBI Accession No. NM 139314.3.

5. The siRNA according to claim 1, wherein the sequence of the siRNA is selected from the sequences of duplex 143, duplex 209, duplex 210, duplex 211, duplex 212, duplex 213, duplex 214, duplex 215, duplex 216, and duplex 217.

6. The siRNA according to claim 1, wherein the siRNA comprises at least one modified nucleotide.

7. The siRNA according to claim 1, wherein all of the nucleotides in the sense strand or the antisense strand are modified nucleotides or nucleotide analogs;
preferably, the modified nucleotide or nucleotide analog is selected from a 2′-methoxy nucleotide, a 2′-fluoro nucleotide, a 2′-deoxy nucleotide, a 2′,3′-seco nucleotide analog, a 2′-fluoroarabino nucleotide, a 2′-methoxyethyl nucleotide, a 2′-amino-modified nucleotide, a 2′-alkyl-modified nucleotide, a 3′-methoxy nucleotide, a 2′-allyl-modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol-modified nucleotide, an abasic nucleotide, a morpholino nucleotide, a threose nucleotide, a locked nucleotide, an unlocked nucleotide, a glycerol nucleotide, or a base-modified nucleotide.

8. The siRNA according to claim 1, wherein the 5′ and 3′ ends of the sense strand independently comprise 1 or 2 phosphorothioate linkages, respectively; and the 5′ and 3′ ends of the antisense strand independently comprise 1 or 2 phosphorothioate linkages, respectively;
preferably, there is a phosphorothioate linkage between nucleotides at positions 1 and 2 from the 5′ end of the sense strand, between nucleotides at positions 2 and 3 from the 5′ end of the sense strand, between nucleotides at positions 1 and 2 from the 3′ end of the antisense strand, between nucleotides at positions 2 and 3 from the 3′ end of the antisense strand, between nucleotides at positions 1 and 2 from the 5′ end of the antisense strand, and between nucleotides at positions 2 and 3 from the 5′ end of the antisense strand.

9. The siRNA according to claim 1, wherein the antisense strand of the siRNA has the following characteristics:
the antisense strand of the siRNA is 23 nucleotides in length, wherein the antisense strand comprises 3 to 10 2′-fluoro nucleotides (preferably at positions 2, 14, and 16; positions 2, 5, 14, and 16; positions 2, 6, 14, and 16; positions 2, 4, 6, 14, and 16; positions 2, 6, 9, 14, and 16; positions 2, 5, 6, 14, and 16; positions 2, 6, 10, 14, and 16; positions 2, 6, 12, 14, and 16; positions 2, 5, 10, 14, and 16; positions 2, 3, 12, 14, and 16; positions 2, 9, 12, 14, and 16; positions 2, 6, 8, 9, 14, and 16; positions 2, 3, 5, 12, 14, and 16; positions 2, 8, 9, 12, 14, and 16; positions 2, 7, 9, 12, 14, and 16; positions 2, 4, 6, 8, 10, 14, 16, 18 and 20; or positions 2, 4, 5, 6, 8, 10, 12, 14, 16, and 18 from the 5′ end), with the remaining positions being 2′-methoxy nucleotides; and the sense strand of the siRNA is 21 nucleotides in length, wherein positions 9, 10, and 11; positions 7, 9, 10, and 11; or positions 5, 7, 9, 10, and 11 from the 5′ end of the sense strand are 2′-fluoro nucleotides, optionally comprising a threose nucleotide at one position (preferably at position 1 from the 5′ end), optionally comprising a 2′-deoxy nucleotide at one position (preferably at position 7 from the 5′ end), with the remaining positions being 2′-methoxy nucleotides; or
the antisense strand of the siRNA is 21 nucleotides in length, wherein the antisense strand comprises 3 to 10 2′-fluoro nucleotides (preferably at positions 2, 6, 14, and 16 from the 5′ end), and the remaining positions are 2′-methoxy nucleotides; the sense strand is 19 nucleotides in length, wherein positions 7, 8, and 9 from the 5′ end of the sense strand are 2′-fluoro nucleotides; or
the antisense strand of the siRNA is 22 nucleotides in length, wherein the antisense strand comprises 3 to 10 2′-fluoro nucleotides (e.g., at positions 2, 6, 14, and 16 from the 5′ end), and the remaining positions are 2′-methoxy nucleotides; the sense strand is 20 nucleotides in length, wherein positions 6, 8, 9, and 10, or positions 8, 9, and 10 from the 5′ end are 2′-fluoro nucleotides;
preferably, the antisense strand of the siRNA is the nucleotide sequence as shown in SEQ ID NO: 143, and the sense strand of the siRNA is the nucleotide sequence as shown in SEQ ID NO: 354.

10. The siRNA according to claim 1, wherein the first nucleotide at the 5′ end of the antisense strand is an (E)-vinylphosphonate-modified nucleotide.

11. The siRNA according to claim 1, wherein the sequence of the siRNA is selected from the sequence of one of the modified duplexes 1 to 21 as shown in Table 3.

12. An siRNA conjugate, comprising the siRNA according to claim 1 and a conjugate molecule;
preferably, the conjugate molecule comprises a linker-targeting ligand, and the linker-targeting ligand comprises N-acetylgalactosamine; more preferably, the linker-targeting ligand is GalNAc (L96).

13. The siRNA conjugate according to claim 12, wherein the siRNA conjugate is selected from conjugates 1 to 242 as shown in Table 2;
preferably, the conjugate is selected from conjugate 1, conjugate 5, conjugate 15, conjugate 16, conjugate 17, conjugate 19, conjugate 21, conjugate 22, conjugate 23, conjugate 28, conjugate 92, conjugate 93, conjugate 120, conjugate 130, conjugate 133, conjugate 225, conjugate 227, conjugate 234, conjugate 235, conjugate 236, and conjugate 238 in Table 2.

14. A pharmaceutical composition, comprising the siRNA according to claim 1 or an siRNA conjugate comprising the siRNA, and a pharmaceutically acceptable carrier;
preferably, the pharmaceutical composition further comprises a second therapeutic agent; more preferably, the second therapeutic agent is an oligonucleotide; further more preferably, the second therapeutic agent is administered in the same or a different medicament as the siRNA or the conjugate.

15. A method for treating or preventing a pathological condition or disease associated with overexpression of angiopoietin-like 4 (ANGPTL4) gene, comprising administering to a subject in need thereof an effective amount of the siRNA according to claim 1, or an siRNA conjugate or pharmaceutical composition comprising the siRNA.

16. The method according to claim 15, wherein the pathological condition or disease is a disease associated with dyslipidemia; preferably, the disease associated with dyslipidemia is hyperlipidemia, hypertriglyceridemia, pancreatitis, familial chylomicronemia syndrome, diabetes, type 2 diabetes, heart disease, myocardial infarction, angina, or atherosclerosis.
